SEQUENCE LISTING (19 sequences)

SEQ ID NO: 1 - protein, 21 aa, Artificial Sequence
  Synthetic
CASSLASRGQGASRNQDTQYF

SEQ ID NO: 2 - protein, 21 aa, Artificial Sequence
  Synthetic
CASSLAGVGQGAGVSGNTLYF

SEQ ID NO: 3 - protein, 21 aa, Artificial Sequence
  Synthetic
CASSLSYQGQGAYQSAETLYF

SEQ ID NO: 4 - protein, 18 aa, Artificial Sequence
  Synthetic
CASSQESQGASHSYEQYF

SEQ ID NO: 5 - protein, 21 aa, Artificial Sequence
  Synthetic
CASGDARQDRGPRCSGNTLYF

SEQ ID NO: 6 - protein, 21 aa, Artificial Sequence
  Synthetic
CASSQDTVGLGGPRSNSDYTF

SEQ ID NO: 7 - protein, 21 aa, Artificial Sequence
  Synthetic
CASSQDTVGLGRPRSNSDYTF

SEQ ID NO: 8 - protein, 14 aa, Artificial Sequence
  Synthetic
CASSLNWSQDTQYF

SEQ ID NO: 9 - protein, 21 aa, Artificial Sequence
  Synthetic
CASSLSLGTGGAHYNYAEQFF

SEQ ID NO: 10 - protein, 21 aa, Artificial Sequence
  Synthetic
CASSLSYQGQEAYQSAETLYF

SEQ ID NO: 11 - DNA, 21 bp, Artificial Sequence
  Synthetic
atctctgcttctgatggctca

SEQ ID NO: 12 - DNA, 21 bp, Artificial Sequence
  Synthetic
tgtggaactggtcatgggaag

SEQ ID NO: 13 - DNA, 21 bp, Artificial Sequence
  Synthetic
agcttttccttggctggagag

SEQ ID NO: 14 - DNA, 21 bp, Artificial Sequence
  Synthetic
agtgttggatacaggccagac

SEQ ID NO: 15 - DNA, 21 bp, Artificial Sequence
  Synthetic
tgcgctcatcttaggctttgt

SEQ ID NO: 16 - DNA, 23 bp, Artificial Sequence
  Synthetic
ttattgctactgggggctacggg

SEQ ID NO: 17 - DNA, 23 bp, Artificial Sequence
  Synthetic
aggcagccagcaccgtatatacc

SEQ ID NO: 18 - DNA, 20 bp, Artificial Sequence
  Synthetic
acgctccctaaccttgcttc

SEQ ID NO: 19 - DNA, 20 bp, Artificial Sequence
  Synthetic
acacaccgaactgttgtcgt

(19) United States
(12) Patent Application Publication
(10) Pub. No.: US 2025/0195644 A1
(43) Pub. Date: Jun. 19, 2025

(54) Compositions For and Methods of Preventing Metastases

(71) Applicant: Duke University, Durham, NC (US)

(72) Inventors: Qi-Jing Li, Durham, NC (US); Laura Christian, Durham, NC (US); Liuyang Wang, Durham, NC (US); Xiao-Fan Wang, Durham, NC (US)

(21) Appl. No.: 18/548,918

(22) PCT Filed: Mar. 4, 2022
(86) PCT No.: PCT/US2022/018960

§ 371 Date: Sep. 4, 2023

Related U.S. Application Data
(60) Provisional application No. 63/157,015, filed on Mar. 5, 2021.

(51) Int. Cl. A61K 39/395; A61K 39/00; A61K 45/06; A61P 35/04; C07K 16/24; C07K 16/28

(52) U.S. Cl. CPC A61K 39/39541; A61K 45/06; A61P 35/04; C07K 16/24; C07K 16/2818; A61K 2039/507; C07K 2317/76

(57) ABSTRACT

Disclosed herein are compositions comprising an interfering molecule and/or an anti-PD1 molecule and using those compositions in methods of preventing or inhibiting metastasis of cancer cells and methods of reducing the risk of developing metastases.

### CROSS-REFERENCE TO RELATED APPLICATIONS

This application claims priority to U.S. Provisional Application No. 63/157,015 filed 5 Mar. 2021, which is incorporated herein in its entirety.

### STATEMENT REGARDING FEDERALLY SPONSORED RESEARCH OR DEVELOPMENT

This invention was made with government support under R33CA225325, R01CA233205, and R01CA225622 awarded by the National Institutes of Health. The government has certain rights in the invention.

### REFERENCE TO THE SEQUENCE LISTING

The Sequence Listing submitted 5 Mar. 2022 as a text file named “22_2039_WO_Sequence_Listing”, created on 5 Mar. 2022 and having a size of 5 kilobytes is hereby incorporated by reference pursuant to 37 C.F.R. § 1.52 (e) (5).

### BACKGROUND

The formation of memory T cells is the hallmark of adaptive immunity, which provides rapid and robust protection against bacteria, virus or tumor during antigen re-encounter. Classically, memory T cells are categorized into two major phenotypes: central memory T cells (TCMs) and effector memory T cells (T_{EM}s) (Sallusto, F., et al. (1999) Nature 401, 708-712). TCMs are long lived, quiescent, and stem-cell like. They bear the chemokine receptor CCR7 and the cell adhesion molecule CD62L, allowing them to enter and patrol secondary lymphoid organs (Unsoeld, H., et al. (2002) Journal of Immunology 169, 638-641; von Andrian, U. H., et al. (2000) New England Journal of Medicine 343, 1020-1034). Upon antigen recognition, they differentiate with multipotent capacity (Williams, M. A., et al. (2007) Annual Review of Immunology 25, 171-192). In contrast, T_{EM}s have a shorter lifespan but are more poised for activation. T_{EM}s circulate and enter peripheral non-lymphoid tissues where they exert effector functions for swift and robust pathogen control (Fuhlbrigge, R. C., et al. (1997) Nature 389, 978-981; Mackay, C. R., et al. (1992) European Journal of Immunology 22, 887-895).

A decade ago, the guardian functions of T_{EM}s in non-lymphoid tissues were re-attributed to a novel non-circulating subset of memory T cells-resident memory T cells (T_{RM}s) (Masopust, D., et al. (2001) Science 291, 2413-2417). Using CD103 (Itgae), a specified integrin molecule that binds E-cadherin on the epithelial barrier (Cepek, K. L., et al. (1994) Nature 372, 190-193), T_{RM}s reside in mucosal tissues and are an integral component of the adaptive immune machinery against viral re-challenge, including vaccinia virus, influenza and herpes-simples virus (HSV) (Ariotti, S., et al. (2014) Science 346, 101-105; Iijima, N., et al. (2015) Trends Immunol 36, 556-564; Jiang, X., et al. (2012) Nature 483, 227-231; Teijaro, J. R., et al. (2011) J Immunol 187, 5510-5514; Wu, T., et al. (2014) J Leukoc Biol 95, 215-224). Upon encountering infected cells, like T_{EM}s, T_{RM}s activate quickly. However, unlike T_{EM}s, T_{RM}s release chemokines to recruit T cells, including circulating T_{EM}s, to the infected tissue for intensified immune protection (Schenkel, Jason M., et al. (2014) Immunity 41, 886-897).

Tumorigenesis is a chronic process and established tumors are composed of a complex microenvironment. Nevertheless, a consensus has been reached that T_{RM}s play an important role in overall anti-tumor immunity. Since tumor metastases presents a much more prominent threat to the patients' life than their primary tumor, there remains an urgent need to establish whether tumor specific T_{RM}s can be induced outside of the primary tumor site, and whether they play protective roles against metastasis.

### BRIEF DESCRIPTION OF THE FIGURES

FIG. 1A-FIG. 1I shows that TCRβ repertoire sequencing identified extensive sharing between tumor and distant mucosa T_{RM}s.

FIG. 1A shows a flow cytometry analysis of CD8⁺CD103⁺ T_{RM}s in the tumor, tumor mucosa, and distant mucosa. One-way ANOVA with Tukey's multiple comparison test, p<0.05. Data were pooled over three independent experiments. FIG. 1B shows an analysis of the unique clonotype number between samples. Wilcoxon signed-rank test, p<0.05. FIG. 1C shows clonal T cell expansion in the tumor and tumor-associated tissues. FIG. 1D is the Shannon Diversity index showing a reduction of CD8⁺ T cell diversity in the tumor, tumor mucosa, and distant mucosa. Wilcoxon signed-rank test, p<0.05. FIG. 1E is a Jaccard similarity analysis comparing distinct T cell clones in the tissues of individual mice. FIG. 1F shows a global T cell similarity analysis that used the Morisita index to measure the presence and abundance of clonotypes between mucosal tissues. Wilcoxon signed-rank test, p<0.05. FIG. 1G shows the Morisita index evaluating clonotype similarity between primary and secondary (metastatic) lung tumor and pre-metastatic lung tissues. Wilcoxon signed-rank text, p<0.05. FIG. 1H shows a Bhattacharyya's Coefficient analysis evaluating the clonal overlap between mammary and lung mucosal tissues. Wilcoxon signed-rank test, p<0.05. FIG. 1H shows an analysis of the 10 most expanded TCRs in the distant mucosa and their overlap between other tissues. FIG. 1J shows the TCR CDR3 sequences of the top 10 most expanded distant mucosa clones listed on the left and their frequency in other tissues is displayed as a percentage. Bars represent mean±s.e.m. and symbols represent individual mice.

FIG. 2A-FIG. 2B show the experimental design for tumor TRM generation and sample collection. FIG. 2A describes experiments using titrating amounts of 4T1 tumor cells and revealed that orthotopic injection of 100 4T1 cells into the mammary fat pad of 4T1 mice caused robust tumor formation at 21 days post-injection. The diagram shows the location of the tumor, tumor mucosa and distant mucosa, the main sites of sample collection. FIG. 2B shows the representative flow cytometry plots and gating of T_{RM}s in the tumor, tumor mucosa, and distant mucosa at day 21 post-tumor cell injection.

FIG. 3 shows that metastatic tumors were not found in the distant mucosa or lung 3 weeks post tumor implantation. Representative H&E images of distant mucosa or lung harvested from 4T1 tumor-bearing mice 3 weeks post-tumor implantation revealed there were no tumor metastases present at this early time-point.

FIG. 4A-FIG. 4D show that single-cell RNA-sequencing revealed intra-tumor T_{Eff/EM} heterogeneity.

FIG. 4A shows independent tSNE plots of sorted live TCRβ⁺CD44⁺CD62L⁻CD69⁻CD103⁻ tumor T_{Eff/EM}, TCRβ⁺CD4⁺CD62L⁺CD69⁻CD103⁻ tumor T_{CM}, TCRβ⁺CD4⁺CD62L⁻ CD69⁺CD103⁺ tumor T_{RM}, and TCRβ⁺CD44⁺CD69⁺CD103⁺ distant mucosa T_{RM} single-cell transcriptomes obtained from 10 tumors and matched distant mucosa. Each dot represents a cell; each color indicates a distinct CD8⁺ T cell cluster. T_{Eff/EM} CD8⁺ clusters were labeled p1-p4. FIG. 4B shows expression of effector molecules and activation markers. FIG. 4C shows expression of transcription factors while FIG. 4D shows expression of proliferation markers in the tumor T_{EFF/EM} clusters.

FIG. 5 shows the sorting strategy for single cell RNA-sequencing sample collection. Representative plots showing the flow cytometry sorting strategy for tumor T_{EM}s, TCMs, T_{RM}s, and distant mucosa T_{RM}s for single-cell RNA-sequencing sample collection. Tumors were harvested 21 days after tumor injection and cells were stained for various cell surface markers as detailed above.

FIG. 6A-FIG. 6E show that the tumor and distant mucosa T_{RM}s were comprised of two distinct subsets that resemble either T_{EM}s or TCMs.

FIG. 6A shows the GeneTrac analysis of surface markers, transcription factors and effector molecules among the major tumor T_{Eff/EM}, T_{CM}, T_{RM}, and distant T_{RM} populations. FIG. 6B is a heatmap of top 100 DEGs between the tumor T_{Eff/EM} and tumor TCMs showing the global gene expression pattern between these circulating memory T cells and active and quiescent T_{RM}s. FIG. 6C is a SCENIC regulon analysis that revealed an enrichment of the top regulons between tumor T_{Eff/EM} and active T_{RM}s and between tumor T_{CM}s and quiescent T_{RM}s. FIG. 6D shows the binary heatmap for top regulons plotted for each cluster. FIG. 6E shows a global enrichment of ribosome genes associated with tumor T_{CM} and quiescent T_{RM} subsets.

FIG. 7A-FIG. 7E shows that tissue environment played a significant role in shaping the formation of T_{RM}s.

FIG. 7A is a AMonocle2 analysis showing the lineage relationship and progression between the four tumor T_{Eff/EM} CD8⁺ subsets and tumor T_{RM}s in contour while FIG. 7B shows the AMonocle2 analysis in a pseudotime plot. The main proliferating population (tumor T_{Eff/EM} p3) was used as the starting point of pseudotime progression in all analyses. FIG. 7C shows the contour analysis and FIG. 7D shows the pseudotime analysis of the tumor T_{Eff/EM} populations, tumor T_{RM}s, and distant T_{RM}s, which demonstrated that these populations distinctly separated by their tissue of origin. The divergence of this lineage path was identified at the branching point. FIG. 7E shows a heatmap of gene expression of the individual T cell subsets approaching and leaving this branch point. 13 critical genes defined this branch point—the majority of which (in red) were signature genes identified in the Th17 lineage. FIG. 7F shows surface markers Lgals3 and Sell, functional molecules GzmB and Wnt10a, and transcription factors Maf and Lef1 projected back on the pseudotime space to exemplify the differences between tumor T_{RM}s (left column) and distant T_{RM}s (right column). FIG. 7G shows that a gene ontology analysis revealed that ribosome pathways were enriched in the distant T_{RM} population while cytokine and chemokine signaling pathways were enriched in tumor T_{RM}s.

FIG. 8 shows that the gene expression of TRM markers confirmed heterogeneity of tumor TRMs. GeneTrac analysis of TRM defining marker Itgae (CD103) is upregulated only in the TRM populations, concomitant with reduced S1pr1 expression.

FIG. 9A-FIG. 9C shows that CXCR6 defined a unique subpopulation of T_{Eff/EMS}.

FIG. 9A is a GeneTrac analysis showing expression levels of chemokine receptors and integrins among all tumor T_{EFF/TEM} subsets. Cxcr6 showed the highest expression among the T_{Eff/EM} p4 population and correlated with lowest S1PR1 expression. FIG. 9B shows that the elevated levels of Cxcr6 in T_{Eff/EM} p4 were associated with enhanced Pdcd1 expression, in opposition to Il7r and Il18r1 expression, which were expressed in T_{Eff/EM} p2. Classical exhaustion markers Nr4a1, Lag3 and Havcr2 were upregulated in the tumor T_{Eff/EM} p4 population. FIG. 9C is a Volcano plot of gene expression between tumor T_{Eff/EM} p2 (left) and T_{Eff/EM} p4 (right) showing that T cell activation markers Gzmb, Klrc1/2, Klrd1 were upregulated in T_{EFF/TEM} p4, indicating it was effector-like. FIG. 9D provides flow cytometry staining on tumor T cells showing protein expression of the two T_{Eff/EM} precursor populations, which were sorted for RNA-seq analysis. FIG. 9E is a PCA analysis that confirmed transcriptional separation of these two populations. FIG. 9F is a gene ontology analysis showing that the CXCR6⁺ population enriched many pathways involving the cell cycle, indicating that this pathway was phenotypically exhausted but highly active.

FIG. 10A-FIG. 10D show that intra-tumoral T_{Eff/EM} p4 cells preferentially expressed CXCR6.

FIG. 10A is a representative flow plot on 4T1 tumor showing that intra-tumor T_{Eff/EMs} expressed more CXCR6 than TRMs. FIG. 10B shows these data graphed by the percentage of CD44+ CD103^{POS/NEG} Cells expressing CXCR6+ and by CXCR6 MFI. I. FIG. 10C is a representative flow plots of 4T1 tumor showing that the intra-tumor TEff/EM p4 population expressed more CXCR6 than T_{Eff/EM} p2. FIG. 10D shows these data graphed by CXCR6 MFI. Symbols represent individual mice and error bars represent mean±s.e.m. p<0.05, Kruskal-Wallis test with multiple comparisons.

FIG. 11A-FIG. 11F provides evidence that CXCR6⁻ T_{Eff/EMS} were the precursor of distant T_{RM}s.

FIG. 11A shows the experimental design of sample collection for TCRβ repertoire sequencing. FIG. 11B is the repertoire analysis showing substantial overlap between high and low frequency TCRs between tumor and distant T_{RM}s. FIG. 11C and FIG. 11D are each a repertoire analysis showing that both CXCR6^{NEG} and CXCR6^{POS} subsets shared TCRs with tumor T_{RM}s and that only the highly expanded clones in the CXCR6^{NEG} population contributed to distant T_{RM} formation. FIG. 11E is the experimental design for T_{Eff/EM} precursor transfer. FIG. 11F shows the preference of CXCR6⁺T_{Eff/EM} precursor cells to stay in the tumor while CXCR6 ⁻T_{Eff/EM} precursors egressed to the distant mucosa. Data is represented as a ratio of recovered cells in the tumor divided by the recovered cells in the distant mucosa or LN. Mann-Whitney test, p<0.05. Bars represent mean±s.e.m. and symbols represent individual mice. Data are pooled over three independent experiments.

FIG. 12A-FIG. 12M show that breaking CXCL16/CXCR6 retention enhanced protection against distant lung tumor metastasis.

FIG. 12A presents the qPCR data for the CXCR6 ligand, Cxcl16, which had the highest expression in the tumor. FIG. 12B shows that Cxcl16 mRNA was similar in the distant mucosa from tumor-bearing mice compared to the mucosa from tumor-naïve mice. Two-way ANOVA with Kruskal-Wallis test, p<0.005. Bars represent mean±s.e.m. and symbols represent individual mice. FIG. 12C shows that flow cytometry validated CXCL16 expression on the surface of 4T1 tumor cells but not mucosal epithelial cells. FIG. 12D shows the confocal microscopy characterization of CXCL16 expression within the 4T1 primary tumor. FIG. 12E shows the experimental design for sample collection in the presence or absence of anti-CXCL16 blocking. FIG. 12F-FIG. 12G shows the global TCRβ repertoire sequencing analysis comparing clonotype shared between the tumor and the pre-metastatic lung in the presence or absence of anti-CXCL16 blocking. FIG. 12F shows these data represented in all frequency categories and FIG. 12G shows zooming into the top 200 high frequency clones. FIG. 12H shows the experimental procedure to monitor spontaneous lung metastasis after surgical removal of the primary tumor in the presence or absence of anti-CXCL16 treatment. FIG. 12I shows the analysis of the primary tumors. Bars represent mean±s.e.m and symbols represent individual mice. Paired t-test, p<0.05. FIG. 12J is an analysis of the number of metastatic tumor nodules. FIG. 12K provides representative images of metastatic tumor lungs. FIG. 12L shows representative H&E staining of anti-CXCL16 and isotype treated lungs showing the area of the lung harboring tumor metastases (dark purple). FIG. 12M provides the quantification of metastatic tumor occupancy in the lung, performed by a third-party in a one-sided blinded manner. Data were measured by diving the area of tumor metastases by total lung area and represented as a percentage. Paired t test, p<0.05. Bars represent mean±s.e.m. and symbols represent individual lung tissue sections. Data represented two individual experiments combined.

FIG. 13A-FIG. 13D show that a combinatorial neoadjuvant therapy enhanced protection against tumor metastasis.

FIG. 13A shows the pre-clinical neoadjuvant therapy schema for the combination of anti-PD1 and anti-CXCL16. Prior to surgery, anti-PD1 therapy was applied to tumor carrying mice to boost the proliferation of PD1+CXCR6+ precursor cells. These precursors were released from the tumor site by blocking CXCL16, the ligand of CXCR6. After surgical resection of the primary tumor, the lung metastasis and overall survival were assessed. FIG. 13B shows the growth of primary tumors before the surgical removal. As the mono-drug, anti-PD1 alone failed to deliver efficacy to 4T1 tumor growth. The anti-PD1 and anti-CXCL16 combinatory treatment had moderate efficacy on the progression of primary tumors. FIG. 13C shows the overall survival of mice through the neoadjuvant therapy. Lung metastasis was the cause of death. As the mono-drug, anti-PD1 alone failed to prolong the survival of post-surgery mice. The anti-PD1 and anti-CXCL16 combinatory treatment increased the overall survival ratio. FIG. 13D shows representative images of 4T1 tumor lung metastases harvested between days 16 to 21 post-surgery. The top panels show the surface view of 4T1 tumor nodules while the bottom panels show the cross-section of the whole lung after HE staining.

### BRIEF SUMMARY

Disclosed herein is a pharmaceutical formulation comprising an interfering molecule that disrupts and/or prevents the physical interaction of CXCL16 with CXCR6; and one or more pharmaceutically acceptable carriers.

Disclosed herein is a pharmaceutical formulation, comprising an interfering molecule that disrupts and/or prevents the physical interaction of CXCL16 with CXCR6; an anti-PD1 molecule; and one or more pharmaceutically acceptable carriers.

Disclosed herein is a hybridoma capable of producing one or more disclosed antibodies.

Disclosed herein are cells used to produce one or more disclosed antibodies.

Disclosed herein are animals used to produce one or more disclosed antibodies.

Disclosed herein is a method of preventing or inhibiting metastasis of cancer cells, the method comprising treating a subject in need thereof by administering to the subject a therapeutically effective amount of an interfering molecule that disrupts and/or prevents the physical interaction of CXCL16 with CXCR6.

Disclosed herein is a method of preventing or inhibiting metastasis of cancer cells, the method comprising treating a subject in need thereof by administering to the subject a therapeutically effective amount of an interfering molecule that disrupts and/or prevents the physical interaction of CXCL16 with CXCR6 and a therapeutically effective amount of an anti-PD1 molecule.

Disclosed herein is a method of preventing or inhibiting metastasis of cancer cells, the method comprising treating a subject in need thereof by administering to the subject a therapeutically effective amount of a pharmaceutical formulation that disrupts and/or prevents the physical interaction of CXCL16 with CXCR6.

Disclosed herein is a method of preventing or inhibiting metastasis of cancer cells, the method comprising treating a subject in need thereof by administering to the subject a therapeutically effective amount of a pharmaceutical formulation that disrupts and/or prevents the physical interaction of CXCL16 with CXCR6, wherein the pharmaceutical formulation comprises an interfering molecule and an anti-PD1 molecule.

Disclosed herein is a method of preventing or inhibiting metastasis of cancer cells, the method comprising administering to a subject in need thereof a neoadjuvant therapy.

Disclosed herein is a method of reducing the risk of developing metastases, the method comprising administering to a subject in need thereof a therapeutically effective amount of an interfering molecule that disrupts and/or prevents the physical interaction of CXCL16 with CXCR6.

Disclosed herein is a method of reducing the risk of developing metastases, the method comprising administering to a subject in need thereof a therapeutically effective amount of an interfering molecule that disrupts and/or prevents the physical interaction of CXCL16 with CXCR6 and a therapeutically effective amount of an anti-PD1 molecule.

Disclosed herein is a method of reducing the risk of developing metastases, the method comprising administering to a subject in need thereof a therapeutically effective amount of a pharmaceutical formulation that disrupts and/or prevents the physical interaction of CXCL16 with CXCR6.

Disclosed herein is a method of reducing the risk of developing metastases, the method comprising administering to a subject in need thereof a therapeutically effective amount of a pharmaceutical formulation that disrupts and/or prevents the physical interaction of CXCL16 with CXCR6, wherein the pharmaceutical formulation comprises an interfering molecule and an anti-PD1 molecule.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

Vectors are known to the art and include viral or non-viral vectors. In an aspect, a disclosed vector can be a non-viral vector, and wherein the non-viral vector comprises a polymer-based vector, a peptide-based vector, a lipid nanoparticle, a solid lipid nanoparticle, or a cationic lipid-based vector. In an aspect, a disclosed vector the vector is can be a viral vector, and the viral vector can comprise an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have cancer, be suspected of having cancer, or be at risk of developing cancer.

As used herein, the term “diagnosed” means having been subjected to an examination by a person of skill, for example, a physician, and found to have a condition that can be diagnosed or treated by one or more of the disclosed interfering molecules, the disclosed anti-PD1 molecules, the disclosed pharmaceutical formulations, or a combination thereof, or by one or more of the disclosed methods. For example, “diagnosed with a disease or disorder” means having been subjected to an examination by a person of skill, for example, a physician, and found to have a condition (such as cancer) that can be treated by one or more of the disclosed interfering molecules, the disclosed anti-PD1 molecules, the disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as cancer) that can likely be treated by one or more of by one or more of the disclosed interfering molecules, the disclosed anti-PD1 molecules, the disclosed pharmaceutical formulations, or a combination thereof, or by one or more of the disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject afflicted with a disease or disorder (e.g., cancer). In an aspect, a patient can refer to a subject that has been diagnosed with or is suspected of having a disease or disorder such as cancer. In an aspect, a patient can refer to a subject that has been diagnosed with or is suspected of having a disease or disorder and is seeking treatment or receiving treatment for a disease or disorder (such as cancer).

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., cancer) based upon an earlier diagnosis by a person of skill and thereafter subjected to treatment for the cancer. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not receiving a disclosed interfering molecule, a disclosed anti-PD1 molecule, a disclosed pharmaceutical formulation, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not receiving a disclosed interfering molecule, a disclosed anti-PD1 molecule, a disclosed pharmaceutical formulation, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-cancer state).

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having cancer). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as cancer). For example, treating a disease or disorder can reduce one or more symptoms of a disease or disorder in a subject by 1%-100% as compared to a control (such as, for example, an individual not having cancer). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder (such as cancer).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as cancer) or related complication from progressing to that complication. In an aspect, preventing metastasis is intended.

As used herein, the terms “administering” and “administration” refer to any method of providing one or more of the disclosed interfering molecules, the disclosed anti-PD1 molecules, the disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed composition, a disclosed pharmaceutical composition, a disclosed therapeutic agent, a disclosed immune modulator, a disclosed proteasome inhibitor, a disclosed small molecule, a disclosed endonuclease, a disclosed oligonucleotide, a disclosed RNA therapeutic, or any combination thereof can comprise administration directly into the CNS or the PNS. Administration can be continuous or intermittent. Administration can comprise a combination of one or more routes.

In an aspect, the skilled person can determine an efficacious dose, an efficacious schedule, and an efficacious route of administration for the disclosed interfering molecules, the disclosed anti-PD1 molecules, the disclosed pharmaceutical formulations, or a combination thereof to treat or prevent a disease or disorder (such as cancer). In an aspect, the skilled person can also alter, change, or modify an aspect of an administering step to improve efficacy of the disclosed interfering molecules, the disclosed anti-PD1 molecules, the disclosed pharmaceutical formulations, or a combination thereof.

By “determining the amount” is meant both an absolute quantification of a particular analyte (e.g., biomarker for cancer, for example) or a determination of the relative abundance of a particular analyte (e.g., a cancer biomarker). The phrase includes both direct or indirect measurements of abundance or both.

As used herein, “modifying the method” can comprise modifying or changing one or more features or aspects of one or more steps of a disclosed method. In an aspect, a method can be altered by changing the amount of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof administered to a subject, or by changing the frequency of administration of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof to a subject, by changing the duration of time that the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof is administered to a subject, or by substituting for one or more of the disclosed components and/or reagents with a similar or equivalent component and/or reagent. The same applies to all disclosed interfering molecules, anti-PD1 molecules, pharmaceutical formulations, anti-chemokines, anti-cancer agents, chemotherapeutics, or all combinations thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

The term “contacting” as used herein refers to bringing one or more of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof together with a target area or intended target area in such a manner that the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof can exert an effect on the intended target or targeted area either directly or indirectly. A target area or intended target area can be one or more of a subject's organs (e.g., lungs, heart, liver, kidney, brain, etc.) hosting cancerous cells. In an aspect, a target area or intended target area can be any cell or any organ infected by a disease or disorder (such as cancer). In an aspect, a target area or intended target area can be any organ, tissue, or cells that are affected by a disease or disorder (such as cancer).

As used herein, “determining” can refer to measuring or ascertaining the presence and severity of a disease or disorder, such as, for example, cancer. Methods and techniques used to determine the presence and/or severity of a disease or disorder are typically known to the medical arts. For example, the art is familiar with the ways to identify and/or diagnose the presence, severity, or both of a disease or disorder (such as, for example, cancer).

As used herein, “effective amount” and “amount effective” can refer to an amount that is sufficient to achieve the desired result such as, for example, the treatment and/or prevention of a disease or disorder (e.g., a cancer) or a suspected disease or disorder. As used herein, the terms “effective amount” and “amount effective” can refer to an amount that is sufficient to achieve the desired an effect on an undesired condition (e.g., a cancer). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a cancer), (ii) attenuates, ameliorates, or eliminates one or more symptoms of the particular disease, condition, or disorder e.g., cancer), or (iii) delays the onset of one or more symptoms of the particular disease, condition, or disorder described herein (e.g., cancer). The specific therapeutically effective dose level for any particular patient will depend upon a variety of factors including the disorder being treated and the severity of the disorder; the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof employed; the disclosed methods employed; the age, body weight, general health, sex and diet of the patient; the time of administration; the route of administration; the rate of excretion of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof employed; the duration of the treatment; drugs used in combination or coincidental with the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof employed, and other like factors well known in the medical arts. For example, it is well within the skill of the art to start doses of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed interfering molecules, the disclosed anti-PD1 molecules, the disclosed pharmaceutical formulations, the disclosed anti-chemokines, the disclosed anti-cancer agents, the disclosed chemotherapeutics, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD. IgE, IgG, and IgM, or subclasses (isotypes) thereof (e.g., IgG1. IgG2, IgG3. IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to—other molecules such as toxins, radioisotopes, etc.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multi-specific antibodies formed from antibody fragments.

A “monoclonal antibody” as used herein refers to homogenous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, by hybridoma, phage selection, recombinant expression, and transgenic animals.

As used herein, the term “humanized antibody” refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues from the complementary determining region (CDR) are replaced by residues from the CDR of a non-human species (e.g., mouse, rat, rabbit, hamster, etc.) that have the desired specificity, affinity, and capability. In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin.

That an antibody “selectively binds” or “specifically binds” to an epitope or receptor means that the antibody reacts or associates more frequently, more rapidly, with greater duration, with greater affinity, or with some combination of the above to the epitope or receptor than with alternative substances, including unrelated proteins. “Selectively binds” or “specifically binds” means, for instance, that an antibody binds to a protein with a KD of about 0.1 mM or less, more usually about 1 μM or less. “Selectively binds” or “specifically binds” means at times that an antibody binds to a protein with a KD of about 0.1 mM or less, at times about 1 μM or less, at times about 0.1 μM or less, at times about 0.01 μM or less, and at times about 1 nM or less. It is understood that, in certain embodiments, an antibody or binding moiety that specifically binds to a first target may or may not specifically bind to a second target. As such, “specific binding” does not necessarily require (although it can include) exclusive binding, e.g., binding to a single target. Thus, an antibody may, in an aspect, specifically bind to more than one target (e.g., human CXCL16 or CXCR6 or PD1 or PDL1). In an aspect, the multiple targets may be bound by the same antigen-binding site on the antibody. For example, an antibody may, in certain instances, comprise two identical antigen-binding sites, each of which specifically binds two or more human Notch receptors (e.g., human CXCL16 or CXCR6 or PD1 or PDL1).

Polyclonal antibodies can be prepared by any known method. Polyclonal antibodies are raised by immunizing an animal (e.g., a rabbit, rat, mouse, donkey, goat, etc.) by multiple subcutaneous or intraperitoneal injections of the relevant antigen (a purified peptide fragment, full-length recombinant protein, fusion protein, etc.) optionally conjugated to keyhole limpet hemocyanin (KLH), serum albumin, etc. diluted in sterile saline and combined with an adjuvant (e.g., Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

As used herein, the term “antibody production” can have both general and specific meanings. In the broad sense, it can refer to the entire process of creating a usable specific antibody, including steps of immunogen preparation, immunization, hybridoma creation, collection, screening, isotyping, purification, and labeling for direct use in a particular method. In the more restricted sense, antibody production refers to the steps leading up to antibody generation but does not include various forms of purifying and labeling the antibody for particular uses. Antibody production involves preparation of antigen samples and their safe injection into laboratory or farm animals to evoke high expression levels of antigen-specific antibodies in the serum, which can then be recovered from the animal. Polyclonal antibodies are recovered directly from serum (bleeds). Monoclonal antibodies are produced by fusing antibody-secreting spleen cells from immunized mice with immortal myeloma cell to create monoclonal hybridoma cell lines that express the specific antibody in cell culture supernatant. Successful antibody production depends upon careful planning and implementation with respect to several important steps and considerations: (i) synthesize or purify the target antigen (e.g., peptide or hapten); (ii) choose an appropriate immunogenic carrier protein; (iii) conjugate the antigen and carrier protein to create the immunogen; immunize animals using appropriate schedule and adjuvant formula; and screen serum (or hybridoma) for antibody titer and isotype (also called antibody characterization).

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, IRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The terms “proliferative disorder” and “proliferative disease” refer to disorders associated with abnormal cell proliferation such as cancer.

“Tumor” and “neoplasm” as used herein refer to any mass of tissue that result from excessive cell growth or proliferation, either benign (noncancerous) or malignant (cancerous) including pre-cancerous lesions. “Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” or “tumor stem cell” or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells” or “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor including both non-tumorigenic cells, which comprise the bulk of the tumor cell population, and tumorigenic stem cells (cancer stem cells).

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the “tumorigenicity” of a tumor refers to the ability of a random sample of cells from the tumor to form palpable tumors upon serial transplantation into immunocompromised mice.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl (9Z,12Z)-octadeca-9,12-dienoate) or another ionizable lipid. In an aspect, the term cationic and ionizable in the context of LNP lipids can be use interchangeably, e.g., wherein ionizable lipids are cationic depending on the pH.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGFIR antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV).).

Administration of an immune modulator can be continuous or intermittent, and administration can comprise a combination of one or more routes.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed interfering molecule, a disclosed anti-PD1 molecule, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a disclosed interfering molecule, a disclosed anti-PD1 molecule, a disclosed pharmaceutical formulation, or a combination thereof) to a subject having or diagnosed with cancer.

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Use in the Disclosed Methods

### 1. Formulations

Disclosed herein is a pharmaceutical formulation comprising an interfering molecule that disrupts and/or prevents the physical interaction of CXCL16 with CXCR6; and one or more pharmaceutically acceptable carriers.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6. CXCL16 is a membrane-bound chemokine that consists of four distinct domains: (i) the chemokine domain, (ii) the mucin-like domain, (iii) the transmembrane domain, and (iv) the ad cytoplasmic domain. After cleavage, soluble CXCL16 acts as a chemoattractant for activated CD8 T cells, NKT cells, and Th1-polarized T cells that express CXCR6. Cleavage can be mediated by a disintegrin and metalloproteinase (ADAM) family protease, ADAM 10.

As known to the art, CXCR6 is a G protein-coupled receptor with seven transmembrane domains that belongs to the CXC chemokine receptor family. The CXCR6 gene, which maps to the chemokine receptor gene cluster, is expressed in several T lymphocyte subsets and bone marrow stromal cells. CXCR6 and its exclusive ligand, chemokine ligand 16 (CCL16), are part of a signaling pathway that regulates T lymphocyte migration to various peripheral tissues (the liver, spleen red pulp, intestine, lungs, and skin) and promotes cell-cell interaction with dendritic cells and fibroblastic reticular cells.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, a disclosed pharmaceutical formulation can comprise an anti-PD1 molecule. In an aspect, a disclosed anti-PD1 molecule can comprise an anti-PD1 antibody, an anti-PDL1 antibody, or any combination thereof.

In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known to those in the art. Similarly, recombinant antibodies and methods of preparing recombinant antibodies are known to those in the art.

In an aspect, a disclosed pharmaceutical formulation can restore one or more aspects of cellular homeostasis and/or cellular functionality and/or metabolic dysregulation in a subject. In an aspect, a disclosed pharmaceutical formulation can restore one or more aspects of cellular homeostasis and/or cellular functionality and/or metabolic dysregulation in a subject having cancer. In an aspect, metabolic dysregulation can be associated with cancer or cancerous cells.

In an aspect, a disclosed pharmaceutical formulation can disrupt and/or prevent the physical interaction of CXCL16 with CXCR6, thereby breaking CXCR6-mediated retention of precursors of resident memory T cells in a primary tumor. In an aspect, when a disclosed pharmaceutical formulation breaks CXCR6-mediated retention of precursors of resident memory T cells, metastasis of cancer cells is prevented or inhibited.

In an aspect, a disclosed pharmaceutical formulation can be used as neoadjuvant therapy.

In an aspect, a disclosed pharmaceutical formulation can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed pharmaceutical formulation can comprise an anti-chemokine therapy that enhances the resident memory T cell formations in tumor-free tissues. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed pharmaceutical formulation can be prepared for systemic or direct administration. In an aspect, a disclosed pharmaceutical formulation can be prepared for oral administration, intravenous administration, intratumoral administration, intraperitoneal administration, or any combination thereof. In an aspect, a disclosed pharmaceutical formulation can be prepared for any method of administration disclosed herein. In an aspect, a disclosed pharmaceutical formulation can be prepared for administration via multiple routes either concurrently or sequentially. For example, in an aspect, a disclosed pharmaceutical formulation can be first administered intratumorally and then be administered intravenously. In an aspect, a disclosed pharmaceutical formulation can be first administered intratumorally and then be administered orally. A skilled clinical can determine the best route of administration for a subject at a given time.

In an aspect, a disclosed antisense molecule or small interfering RNA can refer to the use of oligonucleotides to target RNA, such as RNA relating to CXCL16, CXCR6, PD1, PDL1, or any combination thereof.

In an aspect, a disclosed small molecule can disrupt and/or prevent the physical interaction of CXCL16 with CXCR6. In an aspect, a disclosed small molecule can disrupt and/or prevent the physical interaction of PD1 with PDL1. As known to the art, a disclosed small molecule can include any organic or inorganic material that is not a polymer. As known to the art, a disclosed small molecule can exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition or a disclosed formulation in a disclosed method.

In an aspect, a therapeutically effective amount of a disclosed pharmaceutical formulation can comprise an interfering molecule in a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a therapeutically effective amount of a disclosed pharmaceutical formulation can comprise an anti-PD1 antibody, an anti-PDL1 antibody, or a combination thereof in a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed pharmaceutical formulation can comprise one or more immune modulators. In an aspect, a disclosed pharmaceutical formulation can comprise one or more proteasome inhibitors. In an aspect, a disclosed pharmaceutical formulation can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed pharmaceutical formulation can comprise an anaplerotic agent (such as, for example, C7 compounds like triheptanoin or MCT).

In an aspect, a disclosed pharmaceutical formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme (such as, for example, a missing, deficient, and/or mutant protein or enzyme related to cancer and/or associated with cancerous cells). In an aspect, a disclosed RNA therapeutic can be directed at any protein or enzyme that is overexpressed or is overactive and related to cancer and/or associated with cancerous cells.

In an aspect, a disclosed pharmaceutically acceptable carrier can comprise any disclosed carrier. In an aspect, a disclosed pharmaceutically acceptable carrier can comprise any disclosed excipient.

Disclosed herein is a pharmaceutical formulation, comprising an interfering molecule that disrupts and/or prevents the physical interaction of CXCL16 with CXCR6; an anti-PD1 molecule; and one or more pharmaceutically acceptable carriers.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6. CXCL16 is a membrane-bound chemokine that consists of four distinct domains: (i) the chemokine domain, (ii) the mucin-like domain, (iii) the transmembrane domain, and (iv) the ad cytoplasmic domain. After cleavage, soluble CXCL16 acts as a chemoattractant for activated CD8 T cells, NKT cells, and Th1-polarized T cells that express CXCR6. Cleavage can be mediated by a disintegrin and metalloproteinase (ADAM) family protease, ADAM 10.

As known to the art, CXCR6 is a G protein-coupled receptor with seven transmembrane domains that belongs to the CXC chemokine receptor family. The CXCR6 gene, which maps to the chemokine receptor gene cluster, is expressed in several T lymphocyte subsets and bone marrow stromal cells. CXCR6 and its exclusive ligand, chemokine ligand 16 (CCL16), are part of a signaling pathway that regulates T lymphocyte migration to various peripheral tissues (the liver, spleen red pulp, intestine, lungs, and skin) and promotes cell-cell interaction with dendritic cells and fibroblastic reticular cells.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, a disclosed pharmaceutical formulation can comprise an anti-PD1 molecule. In an aspect, a disclosed anti-PD1 molecule can comprise an anti-PD1 antibody, an anti-PDL1 antibody, or any combination thereof.

In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known to those in the art. Similarly, recombinant antibodies and methods of preparing recombinant antibodies are known to those in the art.

In an aspect, a disclosed pharmaceutical formulation can restore one or more aspects of cellular homeostasis and/or cellular functionality and/or metabolic dysregulation in a subject. In an aspect, a disclosed pharmaceutical formulation can restore one or more aspects of cellular homeostasis and/or cellular functionality and/or metabolic dysregulation in a subject having cancer. In an aspect, metabolic dysregulation can be associated with cancer or cancerous cells.

In an aspect, a disclosed pharmaceutical formulation can disrupt and/or prevent the physical interaction of CXCL16 with CXCR6, thereby breaking CXCR6-mediated retention of precursors of resident memory T cells in a primary tumor. In an aspect, when a disclosed pharmaceutical formulation breaks CXCR6-mediated retention of precursors of resident memory T cells, metastasis of cancer cells is prevented or inhibited.

In an aspect, a disclosed pharmaceutical formulation can be used as neoadjuvant therapy.

In an aspect, a disclosed pharmaceutical formulation can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed pharmaceutical formulation can comprise an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a disclosed pharmaceutical formulation can be prepared for systemic or direct administration. In an aspect, a disclosed pharmaceutical formulation can be prepared for oral administration, intravenous administration, intratumoral administration, intraperitoneal administration, or any combination thereof. In an aspect, a disclosed pharmaceutical formulation can be prepared for any method of administration disclosed herein. In an aspect, a disclosed pharmaceutical formulation can be prepared for administration via multiple routes either concurrently or sequentially. For example, in an aspect, a disclosed pharmaceutical formulation can be first administered intratumorally and then be administered intravenously. In an aspect, a disclosed pharmaceutical formulation can be first administered intratumorally and then be administered orally. A skilled clinical can determine the best route of administration for a subject at a given time.

In an aspect, a disclosed antisense molecule or small interfering RNA can refer to the use of oligonucleotides to target RNA, such as RNA relating to CXCL16, CXCR6, PD1, PDL1, or any combination thereof.

In an aspect, a disclosed small molecule can disrupt and/or prevent the physical interaction of CXCL16 with CXCR6. In an aspect, a disclosed small molecule can disrupt and/or prevent the physical interaction of PD1 with PDL1. As known to the art, a disclosed small molecule can include any organic or inorganic material that is not a polymer. As known to the art, a disclosed small molecule can exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition or a disclosed formulation in a disclosed method.

In an aspect, a therapeutically effective amount of a disclosed pharmaceutical formulation can comprise an interfering molecule in a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a therapeutically effective amount of a disclosed pharmaceutical formulation can comprise an anti-PD1 antibody, an anti-PDL1 antibody, or a combination thereof in a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed pharmaceutical formulation can comprise one or more immune modulators. In an aspect, a disclosed pharmaceutical formulation can comprise one or more proteasome inhibitors. In an aspect, a disclosed pharmaceutical formulation can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed pharmaceutical formulation can comprise an anaplerotic agent (such as, for example, C7 compounds like triheptanoin or MCT).

In an aspect, a disclosed pharmaceutical formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme (such as, for example, a missing, deficient, and/or mutant protein or enzyme related to cancer and/or associated with cancerous cells). In an aspect, a disclosed RNA therapeutic can be directed at any protein or enzyme that is overexpressed or is overactive and related to cancer and/or associated with cancerous cells.

In an aspect, a disclosed pharmaceutically acceptable carrier can comprise any disclosed carrier.

### 2. Hybridomas

Disclosed herein is a hybridoma capable of producing one or more disclosed antibodies. In an aspect, a disclosed hybridoma can produce one or more anti-CXCL16 antibodies. In an aspect, a disclosed hybridoma can produce one or more anti-CXCR6 antibodies. In an aspect, a disclosed hybridoma can produce one or more anti-PDL1 antibodies. In an aspect, a disclosed hybridoma can produce one or more anti-PD1 antibodies. In an aspect, a disclosed hybridoma can produce one or more antibodies for use in a disclosed method.

### 3. Plasmids

Disclosed herein is a plasmid comprising one or more disclosed isolated nucleic acid molecules. Disclosed herein is a plasmid comprising one or more disclosed vectors. Disclosed here are plasmids used in methods of making a disclosed composition such as, for example, a disclosed isolated nucleic acid molecule, a disclosed vector, or a disclosed pharmaceutical formulation. Plasmids and using plasmids are known to the art. In an aspect, disclosed nucleic acid molecule or disclosed plasmids can comprise nucleic acid sequences that encode a Cas endonuclease, that are part of the CRISPR gene editing system, such as, for example, gRNAs directed at CXCL16 and/or CXCR6, or a combination thereof.

### 4. Cells

Disclosed herein are cells used to produce one or more disclosed antibodies. Disclosed herein are cells used to produce one or more disclosed anti-CXCL16 antibodies. Disclosed herein are cells used to produce one or more disclosed anti-CXCR6 antibodies. Disclosed herein are cells used to produce one or more disclosed anti-PD1 antibodies. Disclosed herein are cells used to produce one or more disclosed anti-PDL1 antibodies. Disclosed herein are cells comprising a disclosed isolated nucleic acid molecule, a disclosed vector, and/or a disclosed plasmid. Disclosed herein are cells transduced by a disclosed viral vector. Disclosed herein are cells transfected with a disclosed isolated nucleic acid molecule. Techniques to achieve transfection and transduction are known to the art and using transfected or transduced cells are known to the art. As known to the art, cell lines that can be transformed include carcinoma cell lines. Cell lines that can be used for viral vector production include but are not limited to HEK293 cells, HeLa cells, CHO, stem cell lines, fibroblasts, inducible pluripotent stem cells, primary airway cultures, primary kidney, primary cardiomyocytes, primary neurons, primary hepatocytes, primary myocytes or myotubes, kidney organoids, and brain organoids.

### 5. Animals

Disclosed herein are animals used to produce one or more disclosed antibodies. Disclosed herein are animals used to produce one or more disclosed anti-CXCL16 antibodies. Disclosed herein are animals used to produce one or more disclosed anti-CXCR6 antibodies. Disclosed herein are animals used to produce one or more disclosed anti-PD1 antibodies. Disclosed herein are animals used to produce one or more disclosed anti-PDL1 antibodies.

Disclosed herein are animals treated with one or more disclosed isolated nucleic acid molecules, one or more disclosed vectors, one or more disclosed pharmaceutical formulations, and/or one or more disclosed plasmids. Transgenic animals are known to the art as are the techniques to generate transgenic animals.

### C. Methods of Preventing or Inhibiting Metastasis of Cancer Cells

Disclosed herein is a method of preventing or inhibiting metastasis of cancer cells, the method comprising treating a subject in need thereof by administering to the subject a therapeutically effective amount of an interfering molecule that disrupts and/or prevents the physical interaction of CXCL16 with CXCR6.

Disclosed herein is a method of preventing or inhibiting metastasis of cancer cells, the method comprising treating a subject in need thereof by administering to the subject a therapeutically effective amount of an interfering molecule that disrupts and/or prevents the physical interaction of CXCL16 with CXCR6 and a therapeutically effective amount of an anti-PD1 molecule.

In an aspect, disclosed cancer cells can be in a tumor. In an aspect, disclosed cancer cells can be in one or more tumors. In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6. CXCL16 is a membrane-bound chemokine that consists of four distinct domains: (i) the chemokine domain, (ii) the mucin-like domain, (iii) the transmembrane domain, and (iv) the ad cytoplasmic domain. After cleavage, soluble CXCL16 acts as a chemoattractant for activated CD8 T cells, NKT cells, and Th1-polarized T cells that express CXCR6. Cleavage can be mediated by a disintegrin and metalloproteinase (ADAM) family protease, ADAM 10. As known to the art, CXCR6 is a G protein-coupled receptor with seven transmembrane domains that belongs to the CXC chemokine receptor family. The CXCR6 gene, which maps to the chemokine receptor gene cluster, is expressed in several T lymphocyte subsets and bone marrow stromal cells. CXCR6 and its exclusive ligand, chemokine ligand 16 (CCL16), are part of a signaling pathway that regulates T lymphocyte migration to various peripheral tissues (the liver, spleen red pulp, intestine, lungs, and skin) and promotes cell-cell interaction with dendritic cells and fibroblastic reticular cells.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed interfering molecule can comprise systemic or direct administration. In an aspect, administering a disclosed interfering molecule can comprise oral administration, intravenous administration, intratumoral administration, intraperitoneal administration, or any combination thereof. In an aspect, administering a disclosed interfering molecule can be administered by any method of administration disclosed herein. In an aspect, a disclosed interfering molecule can be administered via multiple routes either concurrently or sequentially. For example, in an aspect, a disclosed interfering molecule can be first administered intratumorally and then be administered intravenously. In an aspect, administering a disclosed interfering molecule can be first administered intratumorally and then be administered orally. A skilled clinician can determine the best route of administration for a subject at a given time.

In an aspect of a disclosed method, a therapeutically effective amount of a disclosed interfering molecule can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of the interfering molecule.

In an aspect, a disclosed method of preventing or inhibiting metastasis can comprise protecting the subject from metastasis. In an aspect, a disclosed method of preventing or inhibiting metastasis can comprise reducing the risk of developing metastasis. In an aspect, a disclosed method of preventing or inhibiting metastasis can comprise treating cancer

In an aspect, a disclosed method can comprise monitoring the subject for adverse effects. In an aspect, in the absence of adverse effects, a disclosed method can comprise continuing to treat the subject. In an aspect, continuing to treat the subject can comprise continuing to administer the interfering molecule. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the interfering molecule administered to the subject, changing the frequency of administration of the interfering molecule, changing the duration of administration of the interfering molecule, changing the route of administration of the interfering molecule, or any combination thereof.

In an aspect, a disclosed method can comprise administering to the subject an anti-PD1 molecule. In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect of a disclosed method, a disclosed anti-PD1 antibody, an anti-PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of the anti-PD1 molecule. In an aspect, a disclosed anti-PD1 molecule can be administered prior to, concurrent with, or after the administration of the interfering molecule.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect, following the administering of a disclosed anti-PD1 molecule, a disclosed method can comprise monitoring the subject for adverse effects. In an aspect, in the absence of adverse effects, a disclosed method can comprise continuing to administer the anti-PD1 molecule. In an aspect, in the presence of adverse effects, a disclosed method can comprise modifying one or more steps of a disclosed method. In an aspect, modifying one or more steps of a disclosed method can comprise modifying the administering step of a disclosed anti-PD1 molecule. In an aspect, modifying the administering step can comprise changing the amount of the anti-PD1 molecule administered to the subject, changing the frequency of administration of the anti-PD1 molecule, changing the duration of administration of the anti-PD1 molecule, changing the route of administration of the anti-PD1 molecule, or any combination thereof.

For example, in an aspect, a disclosed anti-PD1 molecule can be administered about 3 months, about 2 months, or about 1 month prior to the administering of the interfering molecule. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the interfering molecule. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the interfering molecule. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the interfering molecule.

In an aspect, a disclosed method can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, cancer cells can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, preventing or inhibiting metastasis of cancer cells can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment).

In an aspect, a disclosed method can comprise surgically resecting the tumor and/or cancer cells from the subject. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method can be used as neoadjuvant therapy.

In an aspect, a disclosed method can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of preventing or inhibiting metastasis, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body (Carotid |
| | Artery), Ventrolateral Medulla (Medulla) |
| Temperature | Thermosensory neurons (Skin), Preoptic Area |
| | (Hypothalamus) |

In an aspect, a disclosed method of preventing or inhibiting metastasis can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases as well as gRNAs targeting the gene of interest (such as CXCL16 or CXCR6) are known to the art.

In an aspect, a disclosed method of preventing or inhibiting metastasis can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed interfering molecule, a disclosed antibody, or a combination thereof administered to a subject, or by changing the frequency of administration of a disclosed interfering molecule, a disclosed antibody, or a combination to a subject, or by changing the duration of time that a disclosed interfering molecule, a disclosed antibody, or a combination are administered to a subject.

Disclosed herein is a method of preventing or inhibiting metastasis of cancer cells, the method comprising treating a subject in need thereof by administering to the subject a therapeutically effective amount of a pharmaceutical formulation that disrupts and/or prevents the physical interaction of CXCL16 with CXCR6.

Disclosed herein is a method of preventing or inhibiting metastasis of cancer cells, the method comprising treating a subject in need thereof by administering to the subject a therapeutically effective amount of a pharmaceutical formulation that disrupts and/or prevents the physical interaction of CXCL16 with CXCR6, wherein the pharmaceutical formulation comprises an interfering molecule and an anti-PD1 molecule.

In an aspect, disclosed cancer cells can be in a tumor. In an aspect, disclosed cancer cells can be in one or more tumors.

In an aspect, a disclosed pharmaceutical formulation can comprise an interfering molecule and/or an anti-PD1 molecule. In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6 and is discussed supra. As known to the art, CXCR6 is a G protein-coupled receptor with seven transmembrane domains that belongs to the CXC chemokine receptor family and is discussed supra.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed pharmaceutical formulation can comprise systemic or direct administration. In an aspect, administering a disclosed pharmaceutical formulation can comprise oral administration, intravenous administration, intratumoral administration, intraperitoneal administration, or any combination thereof. In an aspect, administering a disclosed pharmaceutical formulation can be administered by any method of administration disclosed herein. In an aspect, a disclosed pharmaceutical formulation can be administered via multiple routes either concurrently or sequentially. For example, in an aspect, a disclosed pharmaceutical formulation can be first administered intratumorally and then be administered intravenously. In an aspect, administering a disclosed pharmaceutical formulation can be first administered intratumorally and then be administered orally. A skilled clinician can determine the best route of administration for a subject at a given time.

In an aspect of a disclosed method, a therapeutically effective amount of a disclosed pharmaceutical formulation can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of a disclosed pharmaceutical formulation.

In an aspect, a disclosed method can comprise monitoring the subject for adverse effects. In an aspect, in the absence of adverse effects, a disclosed method can comprise continuing to treat the subject. In an aspect, continuing to treat the subject can comprise continuing to administer a disclosed pharmaceutical formulation. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of a disclosed pharmaceutical formulation administered to the subject, changing the frequency of a disclosed pharmaceutical formulation administration, changing the duration of a disclosed pharmaceutical formulation administration, changing the route of a disclosed pharmaceutical formulation administration, or any combination thereof.

In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed method can comprise repeating the administering of the anti-PD1 molecule. In an aspect, a disclosed anti-PD1 molecule can be administered prior to, concurrent with, or after the administration of the interfering molecule.

In an aspect, following the administering of a disclosed anti-PD1 molecule, a disclosed method can comprise monitoring the subject for adverse effects. In an aspect, in the absence of adverse effects, a disclosed method can comprise continuing to administer the anti-PD1 molecule. In an aspect, in the presence of adverse effects, a disclosed method can comprise modifying one or more steps of a disclosed method. In an aspect, modifying one or more steps of a disclosed method can comprise modifying the administering step of a disclosed anti-PD1 molecule. In an aspect, modifying the administering step can comprise changing the amount of the anti-PD1 molecule administered to the subject, changing the frequency of administration of the anti-PD1 molecule, changing the duration of administration of the anti-PD1 molecule, changing the route of administration of the anti-PD1 molecule, or any combination thereof.

In an aspect, a disclosed method can comprise administering an interfering molecule one or more times, administering an anti-PD1 molecule one or more times, or administering both an interfering molecule and an anti-PD1 molecule one or more time.

In an aspect, a disclosed method can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, cancer cells can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, preventing or inhibiting metastasis of cancer cells can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., a disclosed pharmaceutical formulation)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., a disclosed pharmaceutical formulation)).

In an aspect, a disclosed method can comprise surgically resecting the tumor and/or cancer cells from the subject. In an aspect, following resecting the tumor and/or cancer cells from the subject, a disclosed method can comprise continuing to administer to the subject a therapeutically effective amount of a disclosed pharmaceutical formulation. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method can be used as neoadjuvant therapy.

In an aspect, a disclosed method can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of preventing or inhibiting metastasis, techniques to monitor, measure, and/or assess the restoring one or more aspects of cellular homeostasis and/or cellular functionality can comprise qualitative (or subjective) means as well as quantitative (or objective) means. These means are known to the skilled person and are discussed supra.

In an aspect, a disclosed method of preventing or inhibiting metastasis comprising administer a disclosed pharmaceutical formulation can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases and gRNAs targeting the gene of interest (such as CXCL16 or CXCR6) are known to the art.

In an aspect, a disclosed method of preventing or inhibiting metastasis comprising administer a disclosed pharmaceutical formulation can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed pharmaceutical formulation, a disclosed interfering molecule, a disclosed antibody, or a combination thereof administered to a subject, or by changing the frequency of administration of a disclosed pharmaceutical formulation, a disclosed interfering molecule, a disclosed antibody, or a combination to a subject, or by changing the duration of time that a disclosed pharmaceutical formulation, a disclosed interfering molecule, a disclosed antibody, or a combination are administered to a subject.

Disclosed herein is a method of preventing or inhibiting metastasis of cancer cells, the method comprising administering to a subject in need thereof a neoadjuvant therapy.

In an aspect, a disclosed neoadjuvant therapy can comprise a therapeutically effective amount of an interfering molecule that disrupts and/or prevents the physical interaction of CXCL16 with CXCR6 and a therapeutically effective amount of an anti-PD1 molecule.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6. CXCL16 is a membrane-bound chemokine that consists of four distinct domains: (i) the chemokine domain, (ii) the mucin-like domain, (iii) the transmembrane domain, and (iv) the ad cytoplasmic domain. After cleavage, soluble CXCL16 acts as a chemoattractant for activated CD8 T cells, NKT cells, and Th1-polarized T cells that express CXCR6. Cleavage can be mediated by a disintegrin and metalloproteinase (ADAM) family protease, ADAM 10. As known to the art, CXCR6 is a G protein-coupled receptor with seven transmembrane domains that belongs to the CXC chemokine receptor family. The CXCR6 gene, which maps to the chemokine receptor gene cluster, is expressed in several T lymphocyte subsets and bone marrow stromal cells. CXCR6 and its exclusive ligand, chemokine ligand 16 (CCL16), are part of a signaling pathway that regulates T lymphocyte migration to various peripheral tissues (the liver, spleen red pulp, intestine, lungs, and skin) and promotes cell-cell interaction with dendritic cells and fibroblastic reticular cells.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed interfering molecule can comprise systemic or direct administration. In an aspect, administering a disclosed interfering molecule can comprise oral administration, intravenous administration, intratumoral administration, intraperitoneal administration, or any combination thereof. In an aspect, administering a disclosed interfering molecule can be administered by any method of administration disclosed herein. In an aspect, a disclosed interfering molecule can be administered via multiple routes either concurrently or sequentially. For example, in an aspect, a disclosed interfering molecule can be first administered intratumorally and then be administered intravenously. In an aspect, administering a disclosed interfering molecule can be first administered intratumorally and then be administered orally. A skilled clinician can determine the best route of administration for a subject at a given time.

In an aspect of a disclosed method, a therapeutically effective amount of a disclosed interfering molecule can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of any aspect of the neoadjuvant therapy.

In an aspect, a disclosed method of preventing or inhibiting metastasis of cancer cells comprising administering a neoadjuvant therapy can comprise monitoring the subject for adverse effects. In an aspect, in the absence of adverse effects, a disclosed method can comprise continuing to treat the subject. In an aspect, continuing to treat the subject can comprise continuing to administer the interfering molecule. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the interfering molecule administered to the subject, changing the frequency of administration of the interfering molecule, changing the duration of administration of the interfering molecule, changing the route of administration of the interfering molecule, or any combination thereof.

In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect of a disclosed method, a therapeutically effective amount of a disclosed anti-PD1 molecule can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of preventing or inhibiting metastasis of cancer cells comprising administering a neoadjuvant therapy can comprise repeating the administering of the anti-PD1 molecule. In an aspect, a disclosed anti-PD1 molecule can be administered prior to, concurrent with, or after the administration of the interfering molecule.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect, following the administering of a disclosed anti-PD1 molecule, a disclosed method of preventing or inhibiting metastasis of cancer cells comprising administering a neoadjuvant therapy can comprise monitoring the subject for adverse effects. In an aspect, in the absence of adverse effects, a disclosed method can comprise continuing to administer the anti-PD1 molecule. In an aspect, in the presence of adverse effects, a disclosed method can comprise modifying one or more steps of a disclosed method. In an aspect, modifying one or more steps of a disclosed method can comprise modifying the administering step of a disclosed anti-PD1 molecule. In an aspect, modifying the administering step can comprise changing the amount of the anti-PD1 molecule administered to the subject, changing the frequency of administration of the anti-PD1 molecule, changing the duration of administration of the anti-PD1 molecule, changing the route of administration of the anti-PD1 molecule, or any combination thereof.

For example, in an aspect, a disclosed anti-PD1 molecule can be administered about 3 months, about 2 months, or about 1 month prior to the administering of the interfering molecule. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the interfering molecule. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the interfering molecule. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the interfering molecule.

In an aspect, a disclosed method of preventing or inhibiting metastasis of cancer cells comprising administering a neoadjuvant therapy can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, cancer cells can comprise ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method of preventing or inhibiting metastasis of cancer cells comprising administering a neoadjuvant therapy, disrupting and/or preventing the physical interaction of CXCL16 with CXCR6 can break CXCR6-mediated retention of precursors of resident memory T cells in a primary tumor. In an aspect of a disclosed method of preventing or inhibiting metastasis of cancer cells comprising administering a neoadjuvant therapy, breaking CXCR6-mediated retention of precursors of resident memory T cells can prevent or inhibit metastasis of cancer cells.

In an aspect, preventing or inhibiting metastasis of cancer cells can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., the neoadjuvant therapy)). In an aspect, preventing or inhibiting metastasis of cancer cells can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as a subject that has not received a disclosed treatment (e.g., the neoadjuvant therapy)).

In an aspect, a disclosed method of preventing or inhibiting metastasis of cancer cells comprising administering a neoadjuvant therapy can comprise surgically resecting the tumor and/or cancer cells from the subject. In an aspect, following resecting the tumor and/or cancer cells from the subject, a disclosed method of preventing or inhibiting metastasis of cancer cells comprising administering a neoadjuvant therapy can comprise continuing to administer to the subject a therapeutically effective amount of the interfering molecule and/or continuing to administer to the subject a therapeutically effective amount of an anti-PD1 molecule.

In an aspect, a disclosed method of preventing or inhibiting metastasis of cancer cells comprising administering a neoadjuvant therapy can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of preventing or inhibiting metastasis of cancer cells comprising administering a neoadjuvant therapy can restore one or more aspects of cellular homeostasis and/or cellular functionality and/or metabolic dysregulation in a subject, such as, for example, a subject having cancer or cancerous cells. In an aspect, a disclosed interfering molecule and/or a disclosed anti-PD1 molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise preventing or inhibiting metastasis of cancer cells in the subject.

In an aspect of a disclosed method of preventing or inhibiting metastasis of cancer cells comprising administering a neoadjuvant therapy, techniques to monitor, measure, and/or assess the restoring one or more aspects of cellular homeostasis and/or cellular functionality can comprise qualitative (or subjective) means as well as quantitative (or objective) means. These means are known to the skilled person and discussed supra.

In an aspect, a disclosed method of preventing or inhibiting metastasis of cancer cells comprising administering a neoadjuvant therapy can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases and gRNAs targeting the gene of interest (such as CXCL16 or CXCR6) are known to the art.

In an aspect, a disclosed method of preventing or inhibiting metastasis of cancer cells comprising administering a neoadjuvant therapy can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of preventing or inhibiting metastasis of cancer cells comprising administering a neoadjuvant therapy can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed interfering molecule, a disclosed antibody, or a combination thereof administered to a subject, or by changing the frequency of administration of a disclosed interfering molecule, a disclosed antibody, or a combination to a subject, or by changing the duration of time that a disclosed interfering molecule, a disclosed antibody, or a combination are administered to a subject.

### D. Methods of Reducing the Risk of Developing Metastases

Disclosed herein is a method of reducing the risk of developing metastases, the method comprising administering to a subject in need thereof a therapeutically effective amount of an interfering molecule that disrupts and/or prevents the physical interaction of CXCL16 with CXCR6.

Disclosed herein is a method of reducing the risk of developing metastases, the method comprising administering to a subject in need thereof a therapeutically effective amount of an interfering molecule that disrupts and/or prevents the physical interaction of CXCL16 with CXCR6 and a therapeutically effective amount of an anti-PD1 molecule.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6 and discussed supra. As known to the art, CXCR6 is a G protein-coupled receptor with seven transmembrane domains that belongs to the CXC chemokine receptor family and discussed supra.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed interfering molecule can comprise systemic or direct administration. In an aspect, administering a disclosed interfering molecule can comprise oral administration, intravenous administration, intratumoral administration, intraperitoneal administration, or any combination thereof. In an aspect, administering a disclosed interfering molecule can be administered by any method of administration disclosed herein. In an aspect, a disclosed interfering molecule can be administered via multiple routes either concurrently or sequentially. For example, in an aspect, a disclosed interfering molecule can be first administered intratumorally and then be administered intravenously. In an aspect, administering a disclosed interfering molecule can be first administered intratumorally and then be administered orally. A skilled clinician can determine the best route of administration for a subject at a given time.

In an aspect of a disclosed method, a therapeutically effective amount of a disclosed interfering molecule can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of reducing the risk of developing metastases can comprise repeating the administering of the interfering molecule.

In an aspect, a disclosed method of reducing the risk of developing metastases can comprise protecting the subject from metastasis. In an aspect, a disclosed method of reducing the risk of developing metastases can comprise preventing or inhibiting metastasis. In an aspect, a disclosed method of reducing the risk of developing metastases can comprise treating cancer.

In an aspect, a disclosed method of reducing the risk of developing metastases can comprise monitoring the subject for adverse effects. In an aspect, in the absence of adverse effects, a disclosed method can comprise continuing to treat the subject. In an aspect, continuing to treat the subject can comprise continuing to administer the interfering molecule. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the interfering molecule administered to the subject, changing the frequency of administration of the interfering molecule, changing the duration of administration of the interfering molecule, changing the route of administration of the interfering molecule, or any combination thereof.

In an aspect, a disclosed method a method of reducing the risk of developing metastases can comprise administering to the subject an anti-PD1 molecule. In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect of a disclosed method, a disclosed anti-PD1 antibody, an anti-PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of reducing the risk of developing metastases can comprise repeating the administering of the anti-PD1 molecule. In an aspect, a disclosed anti-PD1 molecule can be administered prior to, concurrent with, or after the administration of the interfering molecule.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect, following the administering of a disclosed anti-PD1 molecule, a disclosed method of reducing the risk of developing metastases can comprise monitoring the subject for adverse effects. In an aspect, in the absence of adverse effects, a disclosed method can comprise continuing to administer the anti-PD1 molecule. In an aspect, in the presence of adverse effects, a disclosed method can comprise modifying one or more steps of a disclosed method. In an aspect, modifying one or more steps of a disclosed method can comprise modifying the administering step of a disclosed anti-PD1 molecule. In an aspect, modifying the administering step can comprise changing the amount of the anti-PD1 molecule administered to the subject, changing the frequency of administration of the anti-PD1 molecule, changing the duration of administration of the anti-PD1 molecule, changing the route of administration of the anti-PD1 molecule, or any combination thereof.

In an aspect, a disclosed anti-PD1 molecule can be administered about 3 months, about 2 months, or about 1 month prior to the administering of the interfering molecule. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the interfering molecule. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the interfering molecule. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the interfering molecule.

In an aspect, a disclosed method of reducing the risk of developing metastases can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, a subject can have cancer or cancer cells or cancerous cells. In an aspect, a subject can have been diagnosed with cancer and previously received treatment for that cancer. In an aspect, a subject can have ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method of reducing the risk of developing metastases, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, a disclosed method of reducing the risk of developing metastases can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., a disclosed interfering molecule and/or a disclosed anti-PD1 molecule)). In an aspect, a disclosed method of reducing the risk of developing metastases can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., a disclosed interfering molecule and/or a disclosed anti-PD1 molecule)).

In an aspect, a disclosed method of reducing the risk of developing metastases can comprise surgically resecting the tumor and/or cancer cells from the subject. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method of reducing the risk of developing metastases can comprise subjecting the subject to one or more invasive or non-invasive diagnostic assessments. Diagnostic assessments are known to the art. In an aspect, a disclosed non-invasive diagnostic imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method can be used as neoadjuvant therapy.

In an aspect, a disclosed method of reducing the risk of developing metastases can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise reducing the risk of developing metastases in the subject.

In an aspect of a disclosed method of reducing the risk of developing metastases, techniques to monitor, measure, and/or assess the restoring one or more aspects of cellular homeostasis and/or cellular functionality can comprise qualitative (or subjective) means as well as quantitative (or objective) means. These means are known to the skilled person and discussed supra.

In an aspect, a disclosed method of reducing the risk of developing metastases can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases as well as gRNAs targeting the gene of interest (such as CXCL16 or CXCR6) are known to the art.

In an aspect, a disclosed method of reducing the risk of developing metastases can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of reducing the risk of developing metastases can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed interfering molecule, a disclosed antibody, or a combination thereof administered to a subject, or by changing the frequency of administration of a disclosed interfering molecule, a disclosed antibody, or a combination to a subject, or by changing the duration of time that a disclosed interfering molecule, a disclosed antibody, or a combination are administered to a subject.

Disclosed herein is a method of reducing the risk of developing metastases, the method comprising administering to a subject in need thereof a therapeutically effective amount of a pharmaceutical formulation that disrupts and/or prevents the physical interaction of CXCL16 with CXCR6.

Disclosed herein is a method of reducing the risk of developing metastases, the method comprising administering to a subject in need thereof a therapeutically effective amount of a pharmaceutical formulation that disrupts and/or prevents the physical interaction of CXCL16 with CXCR6, wherein the pharmaceutical formulation comprises an interfering molecule and an anti-PD1 molecule.

In an aspect, a disclosed pharmaceutical formulation can comprise an interfering molecule and/or an anti-PD1 molecule. In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6 and is discussed supra. As known to the art, CXCR6 is a G protein-coupled receptor with seven transmembrane domains that belongs to the CXC chemokine receptor family and is discussed supra.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed pharmaceutical formulation can comprise systemic or direct administration. In an aspect, administering a disclosed pharmaceutical formulation can comprise oral administration, intravenous administration, intratumoral administration, intraperitoneal administration, or any combination thereof. In an aspect, administering a disclosed pharmaceutical formulation can be administered by any method of administration disclosed herein. In an aspect, a disclosed pharmaceutical formulation can be administered via multiple routes either concurrently or sequentially. For example, in an aspect, a disclosed pharmaceutical formulation can be first administered intratumorally and then be administered intravenously. In an aspect, administering a disclosed pharmaceutical formulation can be first administered intratumorally and then be administered orally. A skilled clinician can determine the best route of administration for a subject at a given time.

In an aspect of a disclosed method of reducing the risk of developing metastases, a therapeutically effective amount of a disclosed pharmaceutical formulation can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of a disclosed pharmaceutical formulation.

In an aspect, a disclosed method of reducing the risk of developing metastases can comprise monitoring the subject for adverse effects. In an aspect, in the absence of adverse effects, a disclosed method can comprise continuing to treat the subject. In an aspect, continuing to treat the subject can comprise continuing to administer a disclosed pharmaceutical formulation. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the pharmaceutical formulation administered to the subject, changing the frequency of administration of the pharmaceutical formulation, changing the duration of administration of the pharmaceutical formulation, changing the route of administration of the pharmaceutical formulation, or any combination thereof.

In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, following the administering of a disclosed pharmaceutical formulation, a disclosed method can comprise monitoring the subject for adverse effects. In an aspect, in the absence of adverse effects, a disclosed method can comprise continuing to administer a disclosed pharmaceutical formulation. In an aspect, in the presence of adverse effects, a disclosed method can comprise modifying one or more steps of a disclosed method. In an aspect, modifying one or more steps of a disclosed method can comprise modifying the administering step of a disclosed pharmaceutical formulation. In an aspect, modifying the administering step can comprise changing the amount of a disclosed pharmaceutical formulation administered to the subject, changing the frequency of administration of a disclosed pharmaceutical formulation, changing the duration of administration of a disclosed pharmaceutical formulation, changing the route of administration of a disclosed pharmaceutical formulation, or any combination thereof.

In an aspect, a disclosed method can comprise administering a disclosed interfering molecule one or more times, administering a disclosed anti-PD1 molecule one or more times, or administering both a disclosed interfering molecule and a disclosed anti-PD1 molecule one or more times.

In an aspect, a disclosed method of reducing the risk of developing metastases can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride

(Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method of reducing the risk of developing metastases can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, a subject can have cancer or cancer cells or cancerous cells. In an aspect, a subject can have been diagnosed with cancer and previously received treatment for that cancer. In an aspect, a subject can have ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method of reducing the risk of developing metastases, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, reducing the risk of developing metastases can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., a disclosed pharmaceutical formulation)). In an aspect, reducing the risk of developing metastases can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., a disclosed pharmaceutical formulation)).

In an aspect, a disclosed method of reducing the risk of developing metastases can comprise surgically resecting the tumor and/or cancer cells from the subject. In an aspect, following resecting the tumor and/or cancer cells from the subject, a disclosed method can comprise continuing to administer to the subject a therapeutically effective amount of a disclosed pharmaceutical formulation. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method of reducing the risk of developing metastases can comprise subjecting the subject to one or more invasive or non-invasive diagnostic assessments. Diagnostic assessments are known to the art. In an aspect, a disclosed non-invasive diagnostic imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method can be used as neoadjuvant therapy.

In an aspect, a disclosed method of reducing the risk of developing metastases can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise reducing the risk of developing metastases in the subject.

In an aspect of a disclosed method of reducing the risk of developing metastases, techniques to monitor, measure, and/or assess the restoring one or more aspects of cellular homeostasis and/or cellular functionality can comprise qualitative (or subjective) means as well as quantitative (or objective) means. These means are known to the skilled person and are discussed supra.

In an aspect, a disclosed method of reducing the risk of developing metastases can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases as well as gRNAs targeting the gene of interest (such as CXCL16 or CXCR6) are known to the art.

In an aspect, a disclosed method reducing the risk of developing metastases can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of reducing the risk of developing metastases can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed pharmaceutical formulation, a disclosed interfering molecule, a disclosed antibody, or a combination thereof administered to a subject, or by changing the frequency of administration of a disclosed pharmaceutical formulation, a disclosed interfering molecule, a disclosed antibody, or a combination to a subject, or by changing the duration of time that a disclosed pharmaceutical formulation, a disclosed interfering molecule, a disclosed antibody, or a combination are administered to a subject.

### E. Methods of Treating of Cancer

Disclosed herein is a method of treating cancer, the method comprising administering to a subject in need thereof a therapeutically effective amount of an interfering molecule that disrupts and/or prevents the physical interaction of CXCL16 with CXCR6.

Disclosed herein is a method of treating cancer, the method comprising administering to a subject in need thereof a therapeutically effective amount of an interfering molecule that disrupts and/or prevents the physical interaction of CXCL16 with CXCR6 and a therapeutically effective amount of an anti-PD1 molecule.

In an aspect, a disclosed method of treating cancer can comprise preventing or inhibiting metastasis of cancer or cancerous cells in the subject. In an aspect, a disclosed method of treating cancer can comprise reducing the risk of developing metastases in the subject.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6 and discussed supra. As known to the art, CXCR6 is a G protein-coupled receptor with seven transmembrane domains that belongs to the CXC chemokine receptor family and discussed supra.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed interfering molecule can comprise systemic or direct administration. In an aspect, administering a disclosed interfering molecule can comprise oral administration, intravenous administration, intratumoral administration, intraperitoneal administration, or any combination thereof. In an aspect, administering a disclosed interfering molecule can be administered by any method of administration disclosed herein. In an aspect, a disclosed interfering molecule can be administered via multiple routes either concurrently or sequentially. For example, in an aspect, a disclosed interfering molecule can be first administered intratumorally and then be administered intravenously. In an aspect, administering a disclosed interfering molecule can be first administered intratumorally and then be administered orally. A skilled clinician can determine the best route of administration for a subject at a given time.

In an aspect of a disclosed method a method of treating cancer, a therapeutically effective amount of a disclosed interfering molecule can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of treating cancer can comprise repeating the administering of the interfering molecule.

In an aspect, a disclosed method of treating cancer can comprise protecting the subject from metastasis. In an aspect, a disclosed method of treating cancer can comprise preventing or inhibiting metastasis. In an aspect, a disclosed method of treating cancer can comprise reducing the risk of developing metastasis.

In an aspect, a disclosed method of treating cancer can comprise monitoring the subject for adverse effects. In an aspect, in the absence of adverse effects, a disclosed method can comprise continuing to treat the subject. In an aspect, continuing to treat the subject can comprise continuing to administer the interfering molecule. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the interfering molecule administered to the subject, changing the frequency of administration of the interfering molecule, changing the duration of administration of the interfering molecule, changing the route of administration of the interfering molecule, or any combination thereof.

In an aspect, a disclosed method of treating cancer can comprise administering to the subject an anti-PD1 molecule. In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect of a disclosed method of treating cancer, a disclosed anti-PD1 antibody, an anti-PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of treating cancer can comprise repeating the administering of the anti-PD1 molecule. In an aspect, a disclosed anti-PD1 molecule can be administered prior to, concurrent with, or after the administration of the interfering molecule.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect, following the administering of a disclosed anti-PD1 molecule, a disclosed method of treating cancer can comprise monitoring the subject for adverse effects. In an aspect, in the absence of adverse effects, a disclosed method can comprise continuing to administer the anti-PD1 molecule. In an aspect, in the presence of adverse effects, a disclosed method can comprise modifying one or more steps of a disclosed method. In an aspect, modifying one or more steps of a disclosed method can comprise modifying the administering step of a disclosed anti-PD1 molecule. In an aspect, modifying the administering step can comprise changing the amount of the anti-PD1 molecule administered to the subject, changing the frequency of administration of the anti-PD1 molecule, changing the duration of administration of the anti-PD1 molecule, changing the route of administration of the anti-PD1 molecule, or any combination thereof.

For example, in an aspect, a disclosed anti-PD1 molecule can be administered about 3 months, about 2 months, or about 1 month prior to the administering of the interfering molecule. In an aspect, a disclosed anti-PD1 molecule can be administered about 8 weeks, about 7 weeks, about 6 weeks, about 5 weeks, about 4 weeks, about 3 weeks, about 2 weeks, or about 1 week prior to the administering of the interfering molecule. In an aspect, a disclosed anti-PD1 molecule can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to the administering of the interfering molecule. In an aspect, a disclosed anti-PD1 molecule can be administered about 24, 23, 22, 21, 20, 19, 18, 17, 16, 15, 14, 13, 12, 11, 10, 9, 8, 7, 6, 5, 4, 3, 2, or 1 hours prior to the administering of the interfering molecule.

In an aspect, a disclosed method of treating cancer can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a subject can have cancer or cancer cells or cancerous cells. In an aspect, a subject can have been diagnosed with cancer and previously received treatment for that cancer. In an aspect, a subject can have ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method of treating cancer, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, a disclosed method of treating cancer can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., a disclosed interfering molecule and/or a disclosed anti-PD1 molecule)). In an aspect, a disclosed method of treating cancer can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., a disclosed interfering molecule and/or a disclosed anti-PD1 molecule)).

In an aspect, a disclosed method of treating cancer can comprise surgically resecting the tumor and/or cancer cells from the subject. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method of treating cancer can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method can be used as neoadjuvant therapy.

In an aspect, a disclosed method of treating cancer can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise reducing the risk of developing metastases in the subject.

In an aspect of a disclosed method of treating cancer, techniques to monitor, measure, and/or assess the restoring one or more aspects of cellular homeostasis and/or cellular functionality can comprise qualitative (or subjective) means as well as quantitative (or objective) means. These means are known to the skilled person and discussed supra.

In an aspect, a disclosed method of treating cancer can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases as well as gRNAs targeting the gene of interest (such as CXCL16 or CXCR6) are known to the art.

In an aspect, a disclosed method of treating cancer can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of treating cancer can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed interfering molecule, a disclosed antibody, or a combination thereof administered to a subject, or by changing the frequency of administration of a disclosed interfering molecule, a disclosed antibody, or a combination to a subject, or by changing the duration of time that a disclosed interfering molecule, a disclosed antibody, or a combination are administered to a subject.

Disclosed herein is a method of treating cancer, the method comprising administering to a subject in need thereof a therapeutically effective amount of a pharmaceutical formulation that disrupts and/or prevents the physical interaction of CXCL16 with CXCR6.

Disclosed herein is a method of treating cancer, the method comprising administering to a subject in need thereof a therapeutically effective amount of a pharmaceutical formulation that disrupts and/or prevents the physical interaction of CXCL16 with CXCR6, wherein the pharmaceutical formulation comprises an interfering molecule and an anti-PD1 molecule.

In an aspect, a disclosed method of treating cancer can comprise preventing or inhibiting metastasis of cancer or cancerous cells in the subject. In an aspect, a disclosed method of treating cancer can comprise reducing the risk of developing metastases in the subject.

In an aspect, a disclosed pharmaceutical formulation can comprise an interfering molecule and/or an anti-PD1 molecule.

In an aspect, a disclosed interfering molecule can comprise a peptide, a phosphopeptide, peptide fragment, an antibody, an antisense or small interfering RNA, a small molecule, or any combination thereof.

In an aspect, a disclosed interfering molecule can comprise an anti-CXCL16 antibody, an anti-CXCR6 antibody, or any combination thereof. In an aspect, a disclosed interfering molecule can comprise a human anti-CXCL16 antibody, a human anti-CXCR6 antibody, or any combination thereof. Humanized antibodies are known to the art. Anti-CXCL16 antibodies and anti-CXCR6 antibodies are known to the art and can be purchased commercially or can be generated by standard antibody production methodology.

As known to the art, CXCL16 is a ligand for CXC chemokine receptor (CXCR) 6 and is discussed supra. As known to the art, CXCR6 is a G protein-coupled receptor with seven transmembrane domains that belongs to the CXC chemokine receptor family and is discussed supra.

In an aspect, a disclosed anti-CXCL16 antibody can comprise a monoclonal antibody, a humanized monoclonal antibody, or a fragment thereof. In an aspect, a disclosed anti-CXCL16 antibody can comprise a polyclonal antibody, a humanized polyclonal antibody, or a fragment thereof. In an aspect, a disclosed anti-CXCL16 antibody can comprise any antibody or antibody fragment that specifically recognizes CXCL16. In an aspect, a disclosed anti-CXCR6 antibody can comprise a monoclonal antibody, a humanized monoclonal antibody, or a fragment thereof. In an aspect, a disclosed anti-CXCR6 antibody can comprise a polyclonal antibody, a humanized polyclonal antibody, or a fragment thereof. In an aspect, a disclosed anti-CXCR6 antibody can comprise any antibody or antibody fragment that specifically recognizes anti-CXCR6. Antibodies and methods of preparing antibodies are known to those in the art. Similarly, recombinant antibodies and methods of preparing recombinant antibodies are known to those in the art.

In an aspect, administering a disclosed pharmaceutical formulation can comprise systemic or direct administration. In an aspect, administering a disclosed pharmaceutical formulation can comprise oral administration, intravenous administration, intratumoral administration, intraperitoneal administration, or any combination thereof. In an aspect, administering a disclosed pharmaceutical formulation can be administered by any method of administration disclosed herein. In an aspect, a disclosed pharmaceutical formulation can be administered via multiple routes either concurrently or sequentially. For example, in an aspect, a disclosed pharmaceutical formulation can be first administered intratumorally and then be administered intravenously. In an aspect, administering a disclosed pharmaceutical formulation can be first administered intratumorally and then be administered orally. A skilled clinician can determine the best route of administration for a subject at a given time.

In an aspect of a disclosed method of treating cancer, a therapeutically effective amount of a disclosed pharmaceutical formulation can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of treating cancer can comprise repeating the administering of a disclosed pharmaceutical formulation.

In an aspect, a disclosed method of treating cancer can comprise monitoring the subject for adverse effects. In an aspect, in the absence of adverse effects, a disclosed method can comprise continuing to treat the subject. In an aspect, continuing to treat the subject can comprise continuing to administer a disclosed pharmaceutical formulation. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of the pharmaceutical formulation administered to the subject, changing the frequency of administration of the pharmaceutical formulation, changing the duration of administration of the pharmaceutical formulation, changing the route of administration of the pharmaceutical formulation, or any combination thereof.

In an aspect, a disclosed anti-PD1 molecule can comprise an anti-PD1 antibody, an anti-PDL1 antibody, or any combination thereof. In an aspect, a disclosed anti-PD1 antibody can comprise a monoclonal antibody, a humanized monoclonal antibody, or a fragment thereof. In an aspect, a disclosed anti-PD1 antibody can comprise a polyclonal antibody, a humanized polyclonal antibody, or a fragment thereof. In an aspect, a disclosed anti-PD1 antibody can comprise any antibody or antibody fragment that specifically recognizes PD1. In an aspect, a disclosed anti-PDL1 antibody can comprise a monoclonal antibody, a humanized monoclonal antibody, or a fragment thereof. In an aspect, a disclosed anti-PDL1 antibody can comprise a polyclonal antibody, a humanized polyclonal antibody, or a fragment thereof. In an aspect, a disclosed anti-PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, following the administering of a disclosed pharmaceutical formulation, a disclosed method can comprise monitoring the subject for adverse effects. In an aspect, in the absence of adverse effects, a disclosed method can comprise continuing to administer a disclosed pharmaceutical formulation. In an aspect, in the presence of adverse effects, a disclosed method can comprise modifying one or more steps of a disclosed method. In an aspect, modifying one or more steps of a disclosed method can comprise modifying the administering step of a disclosed pharmaceutical formulation. In an aspect, modifying the administering step can comprise changing the amount of a disclosed pharmaceutical formulation administered to the subject, changing the frequency of administration of a disclosed pharmaceutical formulation, changing the duration of administration of a disclosed pharmaceutical formulation, changing the route of administration of a disclosed pharmaceutical formulation, or any combination thereof.

In an aspect, a disclosed method can comprise administering a disclosed interfering molecule one or more times, administering a disclosed anti-PD1 molecule one or more times, or administering both a disclosed interfering molecule and a disclosed anti-PD1 molecule one or more times.

In an aspect, a disclosed method of treating cancer can comprise administering to the subject one or more additional anti-cancer therapies. Anti-cancer therapies are known to the art. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), axitinib (Inlyta), bevacizumab (Avastin), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method of treating cancer can comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL9, CXCL10, CXCL11, CXCL12, CXCL13, CCR2, CCR5, CCR7, CCR8, CCR9, CXCR3, CXCR4, CXCR5, CX3CL1, CX3CR1, or any combination thereof.

In an aspect, a subject can have cancer or cancer cells or cancerous cells. In an aspect, a subject can have been diagnosed with cancer and previously received treatment for that cancer. In an aspect, a subject can have ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

Thus, in an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect of a disclosed method of treating cancer, disrupting and/or preventing the physical interaction of CXCL16 with CXCR6 can break CXCR6-mediated retention of precursors of resident memory T cells in a primary tumor. In an aspect of a disclosed method, breaking CXCR6-mediated retention of precursors of resident memory T cells can prevent or inhibit metastasis of cancer cells.

In an aspect, reducing the risk of developing metastases can comprise a 10%, 20%, 30%, 40%, 50%, 60%, 70%, 80%, 90%, 100%, or any amount of decrease or reduction in the risk of and/or actual metastasis of cancer cells when compared to a control subject (such as, for example, a subject that has not received a disclosed treatment (e.g., a disclosed pharmaceutical formulation)). In an aspect, reducing the risk of developing metastases can comprise a 10-20%, 20-30%, 30-40%, 40-50%, 50-60%, 60-70%, 70-80%, 80-90%, or 90-100% or any amount of decrease or reduction in the risk of and/or actual metastasis of cancer cells when compared to a control subject (such as, for example, a subject that has not received a disclosed treatment (e.g., a disclosed pharmaceutical formulation)).

In an aspect, a disclosed method of treating cancer can comprise surgically resecting the tumor and/or cancer cells from the subject. In an aspect, following resecting the tumor and/or cancer cells from the subject, a disclosed method can comprise continuing to administer to the subject a therapeutically effective amount of a disclosed pharmaceutical formulation. In an aspect, following resecting the tumor and/or cancer cells from the subject, a disclosed method can comprise continuing to administer to the subject a therapeutically effective amount of the interfering molecule and/or continuing to administer to the subject a therapeutically effective amount of an anti-PD1 molecule.

In an aspect, a disclosed method of treating cancer can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect, a disclosed method of treating cancer can be used as neoadjuvant therapy.

In an aspect, a disclosed method of treating cancer can restore one or more aspects of cellular homeostasis and/or cellular functionality and/or metabolic dysregulation in a subject, such as, for example, a subject having cancer or cancerous cells. In an aspect, a disclosed interfering molecule can restore one or more aspects of cellular homeostasis and/or cellular functionality and/or metabolic dysregulation in a subject having cancer. In an aspect, metabolic dysregulation can be associated with cancer or cancerous cells. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a cancer; (vii) reversing, inhibiting, preventing, stabilizing, and/or slowing the rate of progression of a cancer, or (viii) any combination thereof. In an aspect, restoring one or more aspects of cellular homeostasis can comprise improving, enhancing, restoring, and/or preserving one or more aspects of cellular structural and/or functional integrity. In an aspect, restoration can be a partial or incomplete restoration. In an aspect, restoration can be complete or near complete restoration such that the level of expression, activity, and/or functionality is similar to that of a wild-type or control level. In an aspect, restoring one or more aspects of cellular homeostasis and/or cellular functionality can comprise reducing the risk of developing metastases in the subject.

In an aspect of a disclosed method of treating cancer, techniques to monitor, measure, and/or assess the restoring one or more aspects of cellular homeostasis and/or cellular functionality can comprise qualitative (or subjective) means as well as quantitative (or objective) means. These means are known to the skilled person and are discussed supra.

In an aspect, a disclosed method of treating cancer can comprise gene editing one or more relevant genes (such as, for example, genes related to or associated with a protein or enzyme that contributes to, causes, and/or perpetuates cancer or cancerous cells), wherein editing includes but is not limited to single gene knockout, loss of function screening of multiple genes at one, gene knockin, or a combination thereof. In an aspect, for example, a gene editing system such as CRISPR can be used to target CXCL16, CXCR6, or both in a subject's tumor. Cas endonucleases and gRNAs targeting the gene of interest (such as CXCL16 or CXCR6) are known to the art.

In an aspect, a disclosed method of treating cancer can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. CRISPR/Cas9 systems and methods are known to the art.

In an aspect, a disclosed method of treating cancer can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed pharmaceutical formulation, a disclosed interfering molecule, a disclosed antibody, or a combination thereof administered to a subject, or by changing the frequency of administration of a disclosed pharmaceutical formulation, a disclosed interfering molecule, a disclosed antibody, or a combination to a subject, or by changing the duration of time that a disclosed pharmaceutical formulation, a disclosed interfering molecule, a disclosed antibody, or a combination are administered to a subject.

### F. Kits

Disclosed herein is a kit comprising a disclosed interfering molecule, a disclosed anti-PD1 molecule, a disclosed pharmaceutical formulation, or any combination thereof. In an aspect, a kit can comprise a disclosed interfering molecule, a disclosed anti-PD1 molecule, a disclosed pharmaceutical formulation, or any combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to cancer or cancer cells or cancerous cells.

In an aspect, a disclosed kit can comprise at least two components constituting the kit. Together, the components constitute a functional unit for a given purpose (such as, for example, treating a subject diagnosed with or suspected of having a disease or disorder such as cancer). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed interfering molecule, a disclosed anti-PD1 molecule, a disclosed pharmaceutical formulation, or any combination thereof, and a label or package insert with instructions for use. In an aspect, suitable containers include, for example, bottles, vials, syringes, blister pack, etc. The containers can be formed from a variety of materials such as glass or plastic. The container can hold a disclosed interfering molecule, a disclosed anti-PD1 molecule, a disclosed pharmaceutical formulation, or any combination thereof, and can have a sterile access port (for example the container may be an intravenous solution bag or a vial having a stopper pierceable by a hypodermic injection needle). The label or package insert can indicate a disclosed interfering molecule, a disclosed anti-PD1 molecule, a disclosed pharmaceutical formulation, or any combination thereof can be used for treating, preventing, inhibiting, and/or ameliorating a disease or disorder or complications and/or symptoms associated with a disease or disorder such as cancer or metastatic cancer. A kit can comprise additional components necessary for administration such as, for example, other buffers, diluents, filters, needles, and syringes.

In an aspect, a disclosed kit can be used to preventing or inhibiting metastasis of cancer cells. In an aspect, a disclosed kit can be used to risk of developing metastases.

### G. Miscellaneous

Disclosed herein is a method of preventing a cancer metastasis in a subject, the method comprising administering to the subject a therapeutically effective amount of an anti-CXCL16 therapy to the subject such that the cancer metastasis is prevented in the subject.

Disclosed herein is method of treating a cancer metastasis in a subject, the method comprising administering to the subject a therapeutically effective amount of an anti-CXCL16 therapy to the subject such that the cancer metastasis is treated in the subject.

In an aspect, in a disclosed method the anti-CXCL16 therapy can be administered intra-tumorally. In an aspect, a disclosed anti-CXCL16 therapy can be an antibody, small molecule, antisense RNA, a peptide, a polypeptide, aptamer, oligonucleotide, or any combination thereof.

In an aspect, a disclosed anti-CXCL16 therapy can be an antibody.

In an aspect, a disclosed method can further comprise administering one or more additional anti-cancer therapies. In an aspect, a disclosed anti-cancer therapy can comprise surgery, radiation, chemotherapy, or any combination thereof. In an aspect, a disclosed anti-CXCL16 therapy can be administered prior to the one or more additional anti-cancer therapies. In an aspect, a disclosed anti-CXCL16 therapy can be administered concurrently with the one or more additional anti-cancer therapies. In an aspect, a disclosed anti-CXCL16 therapy can be administered after the one or more additional anti-cancer therapies.

### EXAMPLES

Tumorigenesis is a chronic process and established tumors are composed of a complex microenvironment. Consequently, some identified characteristics of tumor T_{RM}s are surprising given the current knowledge of T cell memory. Using scRNA-seq, T_{RM}s in human breast tumors were shown to be similar to terminally differentiated T_{Eff/EMS}: they were Klrc1^{hi}PD-1^{hi}Tim-3^{hi}, but enriched with cytolytic molecules such as Granzyme B (Savas, P., et al. (2018) Nat Med 24, 986-993). T_{RM}s from murine B16 tumors could separate into two populations: one being Blimp1^{hi} and short-lived, similar to the population found in human breast tumors; and the other Id3^{hi}, a marker of long-lived T_{RM} cells (Milner, J. J., et al. (2020) Immunity 52, 808-824.e807). Nevertheless, a consensus has been reached that T_{RM}s play an important role in overall anti-tumor immunity. In mice, the presence of tumor antigen-specific T_{RM}s in the skin, either induced by engineered HSV (Park, S. L., et al. (2018) Nature) or established through previous tumor challenge (Malik, B. T., et al. (2017) Science immunology 2), protected mice from transplanted B16 melanoma. For patients with breast, lung or ovarian cancer, the abundance of CD8⁺CD103⁺ T_{RM}s strongly correlated with longer disease-free survival (Djenidi, F., et al. (2015) J Immunol 194, 3475-3486; Wang, Z.-Q., et al. (2016) Clinical Cancer Research 22, 6290-6297). Since tumor metastases presents a much more prominent threat to the patients' life than their primary tumor, it is critical to establish whether tumor specific T_{RM}s can be induced outside of the primary tumor site, and whether they play protective roles against metastasis.

As a critical machinery for rapid pathogen removal, resident memory T cells (T_{RM}s) are locally generated after initial encounter. However, their development accompanying tumorigenesis remains elusive. Using a murine breast cancer model, T_{RM}s develop in the tumor, the contralateral mammary mucosa, and the pre-metastatic lung. Single-cell RNA-sequencing of T_{RM}s reveals two phenotypically distinct populations representing their active versus quiescent phases. These T_{RM}s in different tissue compartments share the same TCR clonotypes and transcriptomes with a subset of intratumoral effector/effector memory T cells (T_{Eff/EMS}), indicating their developmental ontogeny. Furthermore, CXCL16 is highly produced by tumor cells and CXCR6 TER/EMS are the major subset preferentially egressing the tumor to form distant T_{RM}s. Functionally, releasing CXCR6 retention in the primary tumor amplifies tumor-derived T_{RM}s in the lung and leads to superior protection against metastases. This immunologic fortification implies a new strategy to prevent metastasis in clinical oncology.

While their pivotal roles in the anti-viral response are well appreciated, the ontogeny of T_{RM}s remains elusive. In a study using a skin immunization model and high throughput TCRβ sequencing, T_{CM}s and T_{RM}s were found to develop from the same naïve T cell clone (Gaide, O., et al. (2015) Nat Med 21, 647-653). A single-cell RNA-sequencing (scRNA-seq) study with the acute lymphocytic choriomeningitis virus (LCMV) infection model found that early effector cells with high IL2rα and Ezh2 expression were predisposed to become T_{RM}s (Kurd, N. S., et al. (2020) bioRxiv, 2020.2003.2002.973578). Specifically, Gerlach et al. showed that during LCMV infection, CX3CR1⁻ effector cells were a common precursor of T_{CM}s and T_{RM}s (Gerlach, C., et al. (2016) Immunity 45, 1270-1284). While circulating CX3CR1^{int} peripheral memory cells (T_{PM}s) can also survey the tissues, T_{EM}s arise from CX3CR1^{hi} cells and are prohibited from tissue entry. In both vaccinia virus (VACV) infection and B16 melanoma transplantation models, transferred T_{CM} cells were found to differentiate directly into T_{RM}s (Enamorado, M., et al. (2017) Nature communications 8, 16073). By contrast, in an influenza airway infection model, only transferred T_{EM}s were able to become lung T_{RM}s (Slutter, B., et al. (2017) Science immunology 2). More recently, with a Listeria monocytogenes (LM) infection model, circulating KLRG1⁻ cells are suggested as a common precursor subset for all memory T cell populations including T_{CM}, T_{EM}, CX3CR1^{int} T_{PM}, and T_{RM} (Herndler-Brandstetter, D., et al. (2018) Immunity 48, 716-729). Taken together, these data indicating that the model of T_{RM} induction and their residing tissue environment play a significant role in T_{RM} development.

Employing high-throughput TCRβ repertoire sequencing on a cohort of gastric cancer patients, CD8⁺CD103⁺ T_{RM}s was previously identified in the tumor-adjacent mucosa of gastric tumors. Compared with their peripheral counterparts, tumor infiltrating T cells had a restricted TCRβ repertoire and oligoclonal expansion, resulting in reduced clonotype diversity. Through repertoire analyses of T cells within the adjacent, tumor-free mucosal area, a significant proportion of CD103⁺ T_{RM}s that share the same TCR with highly expanded T cell clones found in the tumor was detected. Furthermore, the diversity of these T cells correlated with patients' long-term prognosis, suggesting their protective role against gastric cancer recurrence and metastasis (Jia, Q., et al. (2015) Oncoimmunology 4, e1001230).

Using a murine breast cancer model, single cell RNA-seq (scRNA-seq) was employed to analyze the heterogeneity of memory T cell subsets in the tumor. the lineage progression of tumor T_{RM}s was a specific focus. Tully differentiated T_{RM}s residing in the tumor and distant (non-tumor-adjacent) mammary mucosal tissues were characterized. Through single cell transcriptome mapping and TCR clonotype tracking, distant mucosal T_{RM}s derived from a particular T cell effector/effector memory intratumoral population that lacked CXCR6 expression were identified. Releasing T cells from primary breast tumors by breaking CXCR6-mediated retention led to enhanced protection against tumor metastasis in the distant lung.

The Examples that follow are illustrative of specific aspects of the invention, and various uses thereof. They set forth for explanatory purposes only and are not to be taken as limiting the invention.

### A. Materials and Methods Employed in Specific Examples

### 1. Animals

Balb/c mice (BALB/cJ) and congenically marked CD45.1⁺ Balb/c mice (CByJ.SJL(B6)−Ptprc^{a}/J0) were purchased from The Jackson Laboratory. Rag2-KO Balb/c mice (C.129S6 (B6)−Rag2^{tm1Fwa} N12) were purchased from Taconic. All mice were housed under pathogen-free conditions and only female mice were used between 6-10 weeks for experimental procedures. Littermates of the same sex were randomly assigned to experimental groups. All mice were used in accordance with Institutional Animal Care and Use Committee guidelines at Duke University.

### 2. Cell Lines, Tumor Model, and Tissue Isolation

The 4T1 mammary carcinoma cell line was a gift from Xiao-Fan Wang (Duke University) and the cell line was authenticated prior to their use in experiments. 4T1 tumor cells were grown in coordination with ATCC guidelines: ATCC-formulated-RPMI-1640 Medium (ATCC 30-2001) was used and supplemented with 10% fetal bovine serum. Cells were grown at 37° C. with 5% CO₂ in 100 mm cell culture dishes (VWR). Cells were sub-cultured at 80% confluence at a ratio of 1:6.

Only female mice were used in all experiments. The 4T1 mammary carcinoma cell line was a gift from Xiao-Fan Wang (Duke University). Tumor cells were harvested by trypsinization and cell viability was evaluated by trypan blue exclusion. 100 4T1 cells in 10 μL of serum-free media were orthotopically injected directly into the mammary gland of anesthetized female Balb/c mice using a micro-syringe with a 26-gauge needle (Hamilton Company, Reno, NV). Tumor progression was closely monitored and tumor growth kinetics were measured. Mice were sacrificed at three weeks post-tumor injection for all tumor-cell sorting experiments and their tumors, tumor mammary mucosa, and contralateral (distant) mammary mucosa tissue were harvested. The primary tumor was first removed, followed by dissection of the remaining mucosa tissue surrounding the tumor (tumor mucosa). Care was taken during the dissection process to ensure that the inguinal lymph nodes were removed prior to mammary mucosa tissue harvest. All tissues were mechanically homogenized and filtered over 70 μm nylon mesh filters (VWR) to obtain a single cell suspension for downstream assays. Enzymatic digestion was avoided as to eliminate the possibility that antibody binding sites could be degraded.

### 3. Characterization of Tumor T_{RM}

To characterize the developmental time-course of tumor T_{RM}s, mice were sacrificed either 2 weeks, 3 weeks, or 4 weeks post-4T1 tumor injection. Tumors were harvested and homogenized into a single suspension. Isolated cells were blocked with anti-mouse CD16/CD32 Fc Block (2.4G2) for 10 minutes prior to antibody staining. Cells were stained with antibodies to CD4 (RM4-5), CD8α (53-6.7), TCRβ (H57-597), CD44 (IM7), CD103 (2E7), CD62L (MEL-14), and CD69 (H1.2F3). All data was acquired on a BD FacsCanto flow cytometer (BD Biosciences) and analyzed using FlowJo software (Treestar).

| Reagent or Resource | Source | Identifier |
|---|---|---|
| Purified Mouse monoclonal | Bio X Cell | Cat# BE0307; |
| anti-CD16/32 | | RRID: AB_2736987 |
| Mouse monoclonal anti-CD4 | Biolegend | Cat# 100510; |
| | | RRID: AB_312713 |
| Mouse monoclonal anti-CD8α | Biolegend | Cat# 100712; |
| | | RRID: AB_312751 |
| Mouse monoclonal | Biolegend | Cat# 109228; |
| anti-TCR1β | | RRID: AB_1575173 |
| Mouse monoclonal | Biolegend | Cat# 109206; |
| anti-TCR1β | | RRID: AB_313429 |
| Mouse monoclonal anti-CD44 | Biolegend | Cat# 103012; |
| | | RRID: AB_312963 |
| Mouse monoclonal | Biolegend | Cat# 121418; |
| anti-CD103 | | RRID: AB_2128619 |
| Mouse monoclonal | Biolegend | Cat# 104428; |
| anti-CD62L | | RRID: AB_830799 |
| Mouse monoclonal anti-CD69 | Biolegend | Cat# 104512; |
| | | RRID: AB_493564 |
| Mouse monoclonal | Biolegend | Cat# 151108; |
| anti-CXCR6 | | RRID: AB_2572145 |
| Mouse monoclonal | Biolegend | Cat# 109104; |
| anti-CD279 | | RRID: AB_313421 |
| Mouse monoclonal | Biolegend | Cat# 132903; |
| anti-IL18R1 | | RRID: AB_2123952 |
| Mouse monoclonal | ThermoFisher | Cat# 15-1271-82; |
| anti-CD127 | | RRID: AB_468793 |
| Mouse monoclonal | Biolegend | Cat# 110708, |
| anti-CD45.1 | | RRID: AB_313497 |
| Mouse monoclonal | Biolegend | Cat# 109830, |
| anti-CD45.2 | | RRID: AB_1186098 |
| Mouse monoclonal | BD Biosciences | Cat# 566740; |
| anti-CXCL16 | | RRID: AB_2869842 |
| Rat monoclonal IgG1κ | Biolegend | Cat# 400408; |
| | | RRID: AB_326514 |
| Cy3 AffiniPure Goat Anti-Rat | Jackson | Cat# 112-165-175; |
| IgG | ImmunoResearch | RRID: AB_2338252 |
| Mouse monoclonal CXCL16 | Leinco | Cat# C1430; |
| | Technologies | RRID: AB_2828491 |
| Rat IgG2a isotype control | Bio X Cell | Cat# BE0089 |

### 4. Sequencing of TCRβ Repertoire

Tumor, tumor-adjacent mammary mucosa, contralateral mammary mucosa, draining lymph nodes, and spleen were isolated from 4T1 tumor bearing mice at 3-weeks post-tumor injection and lysed in TRIzol Reagent (Sigma Aldrich). RNA was extracted using the Direct-zol RNA kit (Zymo Research) according to the manufacturer's instructions. cDNA was synthesized using the qScript Flex cDNA synthesis kit (Quanta Biosciences) with a constant region-specific primer (5′-ATCTCTGCTTCTGATGGCTCA-3′ (SEQ ID NO:11)). Multiplex PCR was performed to amplify the CDR3 region of rearranged TCRβ loci and a set of primers, each specific to a specific TCR VB segments, and a reverse primer to the constant region of TCRβ were used to generate a library of amplicons that cover the entire CDR3 region. PCR products were loaded on agarose gels and bands between 220-240 bp were extracted and purified using the QIAquick Gel Extraction kit (QIAGEN). These purified products were sequenced using the Illumina HiSeq X Ten machine.

### 5. Analysis of TCRβ Repertoire

Sequence data were analyzed with MiXCR (v3.06) (Bolotin, D. A., et al. (2015) Nature Methods 12, 380-381). This software first aligned sequence short reads to reference T cell receptors, then extracted CDR3 sequences, and exported TCR clonotypes. The tcR package under the R computing environment was used for clonotype summary, repertoire diversity, and similarity analysis. In these analyses, out-of-frame TCR clonotypes were excluded (Nazarov, V. I., et al. (2015) BMC Bioinformatics 16, 175). Figures were plotted using R ggpubr.

### 6. Preparation and Sequencing of the 10× Genomics Library

Libraries were prepared following the 10× Genomics Single Cell 3′ protocol. Single cells were dissociated, washed, and resuspended in a 1×PBS/0.04% BSA solution at a concentration of 1000 cells/μL to remove dead cells and contaminants. A Cellometer (Nexcelom) was used to determine cell viability and cells were normalized to 1×10⁶ cells/mL. Cells were then combined with a master mix including reverse transcription reagents. With this, gel beads carrying the Illumina TruSeq Read 1 sequencing primer, a 16 bp 10× barcode, a 12 bp unique molecular identifier (UMI), and a poly-dT primer were loaded onto the chip, together with oil for the emulsion reaction. Reverse transcription occurs in nanoliter-scale gel beads in emulsion (GEMs) so that all cDNAs within a GEM share a common barcode. After this reverse transcription reaction, the GEMs were broken and full-length cDNA purified with Silane Dynabeads and SPRI beads then assayed on an Agilent 4200 TapeStation High Sensitivity D5000 ScreenTape (Santa Clara, CA) for qualitative and quantitative analysis. Illumina P5 and P7 sequences (San Diego, CA), a sample index and TruSeq read 2 primer sequences were added via End Repair, A-tailing, Adaptor Ligation and PCR. Sequences were generated using paired end sequencing on an Illumina sequencing platform at a minimum of 50,000 reads/cell.

### 7. Single-Cell RNA-Seq Analysis

Raw short reads were demultiplexed, filtering and mapped to mouse genome GRCm38/mm10 using cellranger v2.02. The gene count matrices from cellranger were subjected to quality control, pre-processing and clustering using the R Seurat 2.3.4 package (Butler, A., et al. (2018) Nature biotechnology 36, 411-420). Low-quality cells that had less than 200 expressed genes and more than 5% mitochondrial genes were filtered out. Gene counts were scaled to total gene expression and percentage of mitochondrial genes with a scaling factor of 10,000, and then log-transformed. The high dimensional data for each sample were reduced by PCA and t-Distributed Stochastics Neighbor Embedding (tSNE). The FindCluster function was used to group clusters in each sample with a resolution of 0.6. Differential expressed genes (DEGs) were identified using the Wilcoxon rank-sum test. The python package scanpy v 1.4.1 (Wolf, F. A., et al. (2018) Genome biology 19, 15) was used to integrate analysis for all samples following the common procedure. The expression matrix was normalized and log transformed through scanply.pp.loglp function, and high variable genes were chosen by scanpy.pp.highly_variable_genes. SCENIC (Aibar, S., et al. (2017) Nat Methods 14, 1083-1086) was used to perform the gene regulatory network (regulon) analysis. This algorithm contained three major steps: (1) find the gene co-expression modules between transcription factors and target genes using GENIE3 (R package); (2) identify co-expression modules between cis-regulatory motif and their target genes using RcisTarget; and (3) score each regulon through AUCell to select the top regulons in each sample.

For the Genetrac analysis, which plots the gene expression values in each measured cell across pre-defined groups, the scanpy toolkit was used but did not use the option of hierarchical clustering, so the cells within each group were ordered randomly. To infer the lineage development of T_{EFF/EM} to T_{CM}, Monocle package v2.5.4 was used (Qiu, X., et al. (2017) Nat Methods 14, 979-982). The top 2000 significant DEGs were chosen to order genes for trajectory reconstruction using the DDRTree method followed by dimension reduction, cell trajectory inference, and pseudo-time measurements, which were computed via reversed graph embedding.

### 8. Bulk RNA-Sequencing

Tumors were harvested from 4T1 tumor bearing mice and homogenized into a single cell suspension. Isolated cells were stained with a LIVE/DEAD Fixable Aqua stain (Thermo Fisher) and blocked with anti-mouse CD16/CD32 Fc Block (2.4G2) for 10 minutes prior to antibody staining. Cells were stained with antibodies to CD8× (53-6.7), TCRβ (H57-597), CD44 (IM7), CD127 (A7R34), IL18R (BG/IL18RA), CD279 (RMPI-30), and CXCR6 (SA051D1) and sorted using the MoFlo Astrios cell sorter. Depending on the experimental yield for 4 replicates, 2.5×10⁴-5×10⁴ of each T_{EM} precursor population were sorted and directly lysed in Lysis Buffer. RNA was extracted using the RNAqueous Micro RNA Isolation Kit (Thermo Fisher) according to the manufacturer's instructions. RNA samples were evaluated for concentration by Qubit (Thermo Fisher) and integrity using an Agilent 2100 Bioanalyzer. Clontech Ultra low libraries were prepared and sequenced using the Illumina HiSeq platform.

### 9. Bulk RNA-Sequencing Analysis

High-throughput short reads were trimmed, filtered, mapped, and counted. The sequence quality was assessed using fastqc software (Andrews 2010) and filtered using fastp (PMID: 30423086 or Chen et al. 2018). Reads were mapped to mouse reference M23 (Genecode) using STAR software (PMID: 23104886 or Dobin et al. 2013). For RNA-seq, read counts per gene were generated using featureCounts (PMID: 24227677 or Liao et al. 2014). DESeq2 was used to perform counts normalization and test for differential expressed genes (Love et al 2014). Gene ontology (GO) enrichment analysis was computed using ClusterProfiler (Yu et al. 2013).

### 10. Adoptive Transfer of T_{EM} Precursor Cells

Tumors were harvested from female CD45.1⁺ Balb/c mice bearing 3-week established 4T1 tumors and homogenized into a single cell suspension. Isolated cells were stained with a LIVE/DEAD Fixable Aqua stain (Thermo Fisher) and blocked with anti-mouse CD16/CD32 Fc Block (2.4G2) for 10 minutes prior to antibody staining. Cells were stained with antibodies to CD8α (53-6.7), TCRβ (H57-597), CD44 (IM7), CD127 (A7R34), IL18R (BG/IL18RA), CD279 (RMPI-30), and CXCR6 (SA051D1) and sorted using the MoFlo Astrios cell sorter. Then, 1.5×10⁴-2×10⁴ cells of each T_{EM} precursor population were sorted depending on the yield of the individual experiment. These cells were directly transferred orthotopically into the mammary gland of 2-week tumor-bearing Rag2KO Balb/c mice. Two weeks later, animals were sacrificed and stained with antibodies to CD8α (53-6.7), TCRβ (H57-597), CD44 (IM7), CD103 (2E7), CD45.1 (A20) and CD45.2 (104) for flow cytometry analysis on BD FACSCanto flow cytometers (BD Biosciences) and analyzed with FlowJo software (Treestar).

11. qPCR of CXCL16

Tumor, tumor-adjacent mammary mucosa, contralateral mammary mucosa, draining lymph nodes, and spleen were isolated from 4T1 tumor bearing mice and lysed in TRIzol Reagent (Sigma Aldrich). RNA was extracted using the Direct-zol RNA kit (Zymo Research) according to the manufacturer's instructions. After annealing oligo-DT primers, cDNA was synthesized using the qScript Flex cDNA synthesis kit (Quanta Biosciences), according to the manufacturer's instructions. SYBR-Green based real-time PCR method was used to quantify the relative expression of Cxcl16 mRNA and three housekeeping genes: Hprt, Sdha, and Ywhaz.

| Target | Sequence | SEQ ID NO. |
|---|---|---|
| Cxcl16-F | TGTGGAACTGGTCATGGGAAG | SEQ ID NO: 12 |
| Cxcl16-R | AGCTTTTCCTTGGCTGGAGAG | SEQ ID NO: 13 |
| Hprt-F | AGTGTTGGATACAGGCCAGAC | SEQ ID NO: 14 |
| Hprt-R | TGCGCTCATCTTAGGCTTTGT | SEQ ID NO: 15 |
| Sdha-F | TTATTGCTACTGGGGGCTACGGG | SEQ ID NO: 16 |
| Sdha-R | AGGCAGCCAGCACCGTATATACC | SEQ ID NO: 17 |
| Ywhaz-F | ACGCTCCCTAACCTTGCTTC | SEQ ID NO: 18 |
| Ywhaz-R | ACACACCGAACTGTTGTCGT | SEQ ID NO: 19 |

### 12. Flow Cytometry of CXCL16

4T1 tumor cells were cultured for 24 hours in media containing 20 ng/ml TNFα and 20 ng/mL IFNγ cytokines (Peprotech) in the presence of 1 μM ADAM10 inhibitor to prevent membrane shedding (GI254023X, Sigma). Cells were dissociated with an enzyme-free dissociation buffer and homogenized to a single cell suspension. Mucosal epithelial cells were harvested directly from a tumor naïve Balb/c mouse and were mechanically homogenized and filtered over 70 μm nylon mesh filters (VWR) to obtain a single cell suspension. Isolated cells were stained with a LIVE/DEAD Fixable Aqua stain (Thermo Fisher) and blocked with anti-mouse CD16/CD32 Fc Block (2.4G2) for 10 minutes prior to antibody staining. Cells were stained with either CXCL16 antibody (12-81, BD Biosciences) or isotype control (Rat IgG1κ, Biolegend). All data was acquired on a BD FacsCanto flow cytometer (BD Biosciences) and analyzed using FlowJo software (Treestar).

### 13. Confocal Microscopy

Fresh tissue samples from 4T1 tumors were cryosectioned into 20 μm sections followed by immediate fixation in 4% PFA for 20 minutes. Tissue slides were immunostained with a primary antibody against CXCL16 (12-81, BD Biosciences) followed by a Cy 3 Goat Anti-Rat IgG secondary antibody (Jackson ImmunoResearch), along with control slides which did not receive secondary antibody stain. Microscopic images were acquired with a confocal microscope (Carl Zeiss 710 inverted) using a 20× objective. Images were construction with z-stacks of images using Zen imaging software (Zeiss).

14. Antibody Blocking Experiments with Lung H&E Analysis

Balb/c mice bearing 4T1 tumors were intratumorally injected with either 100 μg anti-CXCL16 (142417, Leinco Technologies), with or without 200 μg anti-Thy 1.2 (30H12, BioXCell) or 500 μg IgG2a isotype antibody (2A3, BioXCell) at days 7, 14, and 21 post-tumor injection. Primary tumors were surgically removed at day 25 and mice were monitored for their humane endpoint. At this time, lungs were harvested and placed into Bouin's solution fixative for 48 hours for visualization and quantification of lung tumor nodules. A representative subset of lungs (n=3) were placed in a 70% ethanol aqueous solution and routine hematoxylin and eosin (H&E) staining was performed. Each sample was sectioned into 5 slices and independently analyzed by three oncologists to evaluate the total area of the lung harboring tumor metastases. The metastatic tumor area and total lung area were first calculated separately, then the tumor area was divided by total lung area. The resultant data were represented as a percentage of the area of the lung harboring metastases. All image processing analysis was performed in ImageJ software (NIH).

### 15. Quantification and Statistical Analysis

All statistical analysis was carried out using GraphPad v7.03 and R programming language. Specific statistical methods used for analysis are detailed in the text. A p-value of less than 0.05 was set as statistically significant.

### B. SPECIFIC EXAMPLES

### Example 1

### Resident Memory T Cells Develop in Tumor and Distant Tumor-Free Mucosal Tissues

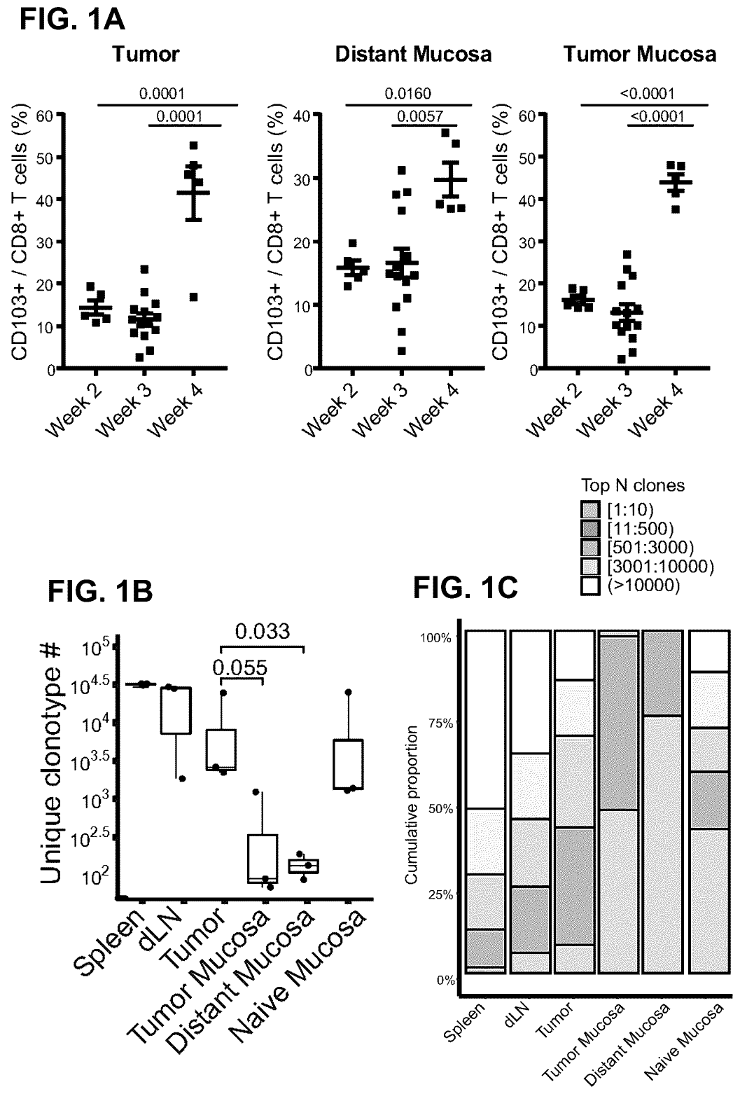

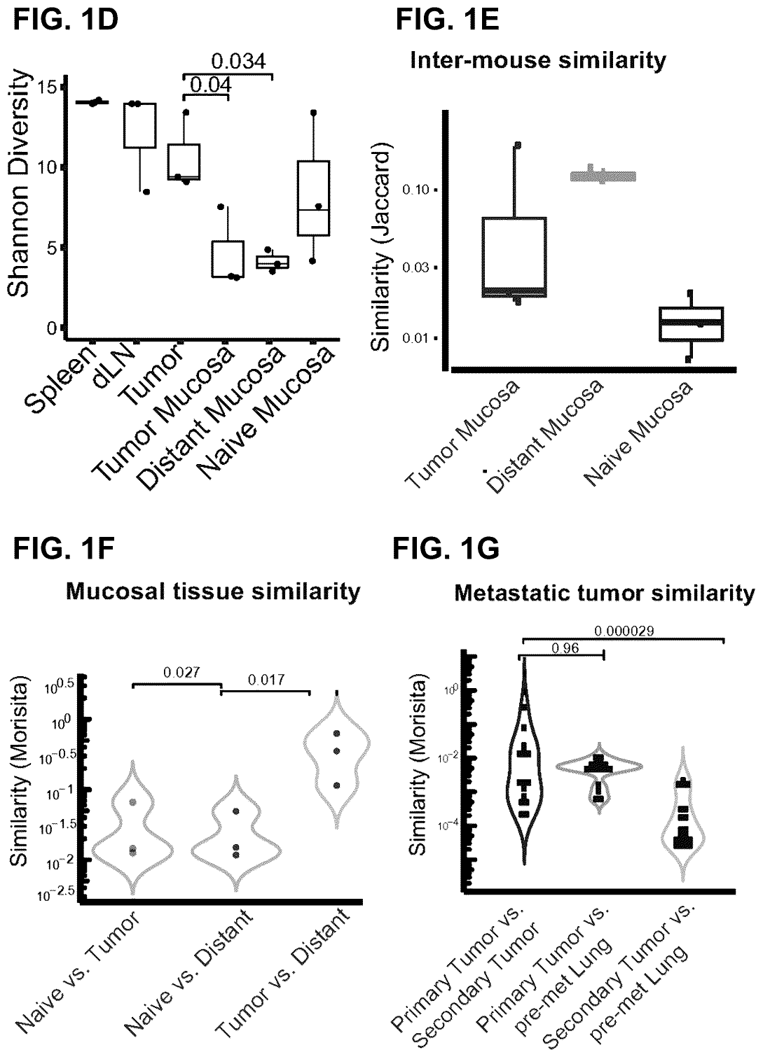

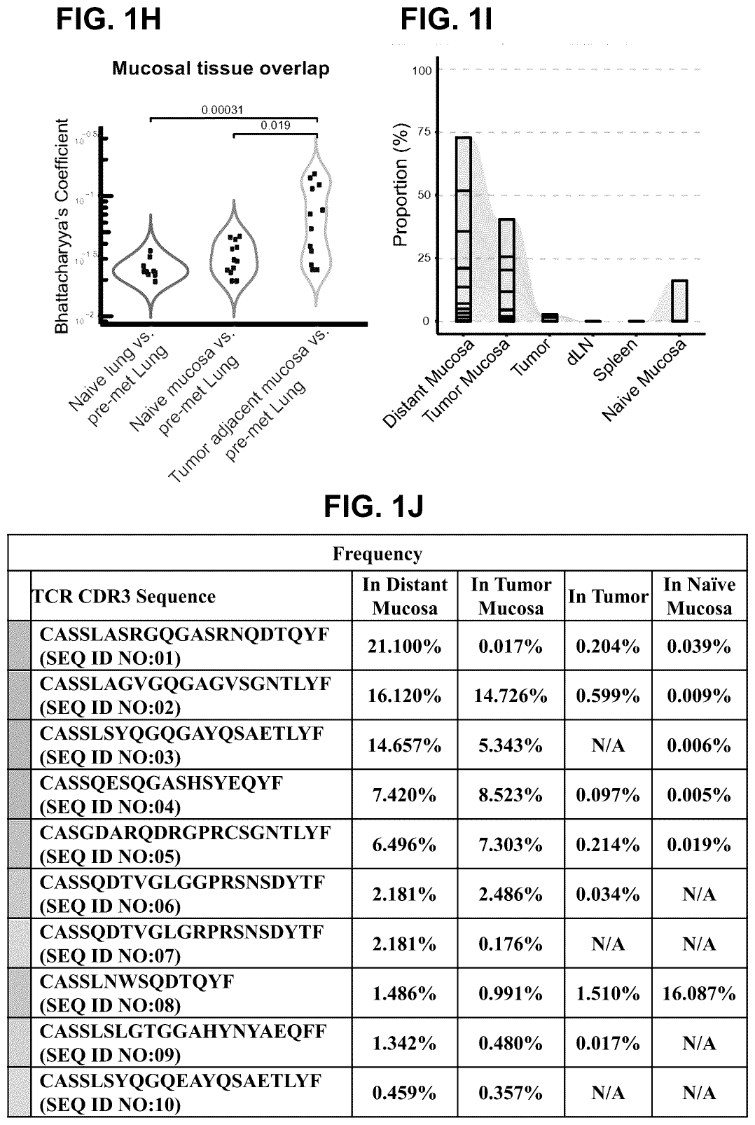

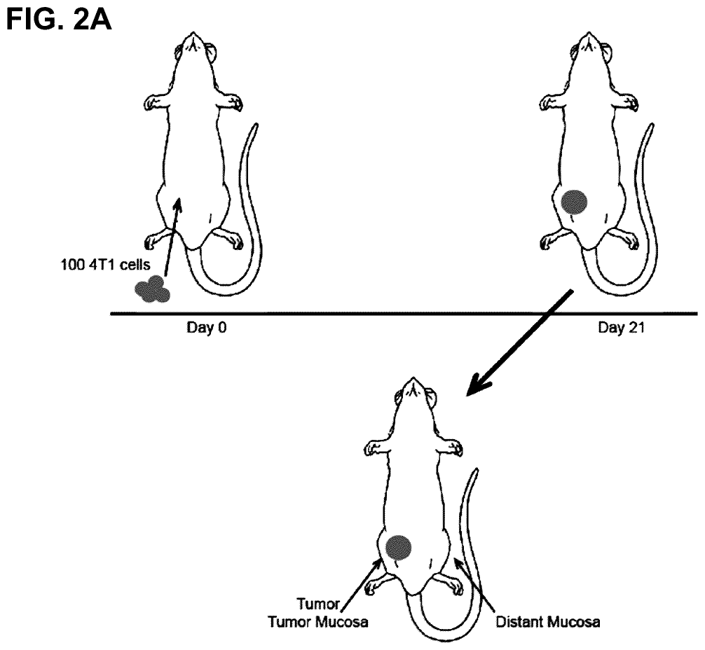

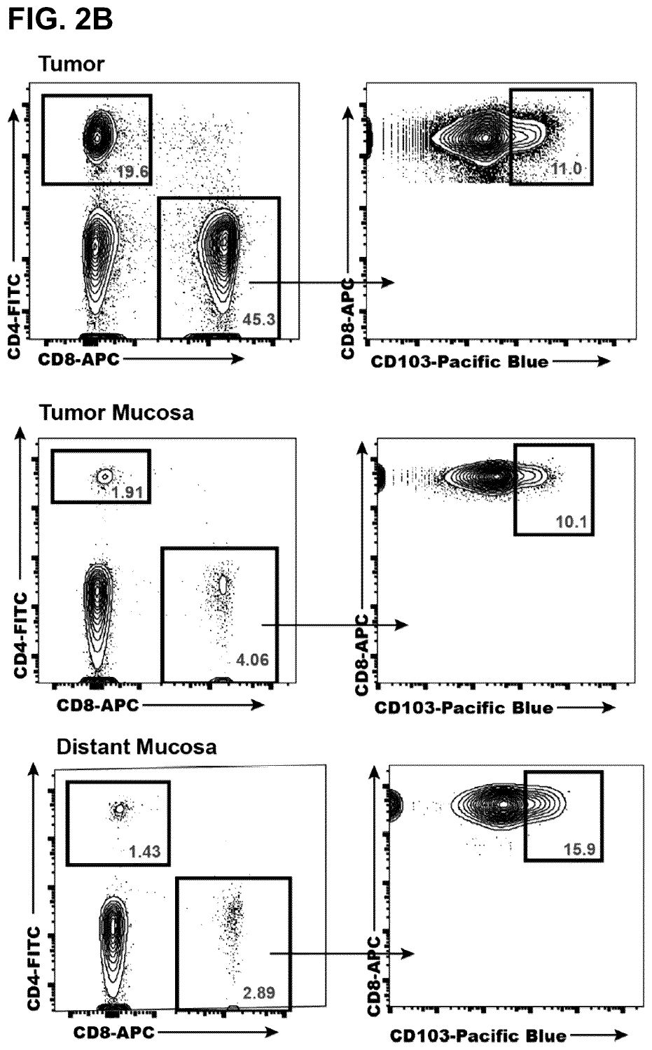

To study tumor-resident memory CD8⁺ T cells, the orthotopic 4T1 triple negative breast tumor model was used. Tumor-inducing doses of 4T1 cells were titrated and it was determined that implanting 100 4T1 cells into the mammary fat pad had 100% penetrance to form primary tumors in 3 weeks, without visibly detectable lung metastases until 5 weeks (FIG. 2A). At different time-points post-tumor inoculation, the tumor and tumor-adjacent mucosa were harvested to assess the development of tumor T_{RM}s (FIG. 2B). The distant (tumor-free) mammary gland mucosa were initially included as a T_{RM}-free control, but CD8⁺CD103⁺ T_{RM}s developed in all tissues and their frequency increased with tumor growth, even in the distant mucosa (FIG. 1A).

To assess the overall structure of the T cell repertoire within different compartments, TCRβ deep sequencing was performed without T cell purification and on multiple tissues collected from tumor-bearing mice. The mammary gland from tumor-naïve mice was included as a control (Table 1). Within the tissues from tumor-bearing mice, using the spleen as the standard, the number of unique TCR clonotypes was lower in the tumor and further contracted in the tumor-adjacent and distant mammary gland mucosa (FIG. 1B). This clonotype contraction was accompanied by oligoclonal expansion. This means that around 10,000 different TCR clones occupied about 50% of the repertoire space in the spleen. Aa similar amount of space was occupied by slightly more than 500 clonotypes in the tumor and 10 clonotypes in the tumor-adjacent mucosa. Most strikingly, 10 different clones of T cells accounted for 75% of the total T cell repertoire within the distant mucosa (FIG. 1C). Accordingly, when the TCR diversity was measured by Shannon entropy, which is determined by both the clonotype number and expansion level of individual clonotypes, T cell diversity was reduced in the tumor, tumor-adjacent mucosa, and tumor-distant mucosa as a cascade (FIG. 1D). Also found in the mammary gland mucosa of female monkeys, the mammary mucosa of tumor-naïve mice harbored T cells (Sircar, P., et al. (2010) Journal of Immunology 185, 2191-2199). However, T cells residing in this environment have a much more diverse repertoire compared to tumor-bearing mice (FIG. 1B, FIG. 1C, and FIG. 1D). In sum, these data indicate that, in tumor-bearing mice, T cell residency within the mammary gland mucosa selects for a limited population of T cells.

TABLE 1
QC Analysis of TCRβ Repertoire Sequencing
Samples related to FIG. 1A-FIG. 1J.

| | Total | Total Mapped | Unique TCR | Mean Read |
|---|---|---|---|---|
| Sample ID | RNA | Reads | Clonotypes | Per TCR |
| Tumor 1 | 500 ng | 84329 | 2626 | 32.11 |
| Tumor 2 | 500 ng | 37368 | 2236 | 16.71 |
| Tumor 3 | 500 ng | 82313 | 24636 | 3.34 |
| Tumor Mucosa 1 | 500 ng | 16869 | 91 | 185.37 |
| Tumor Mucosa 2 | 500 ng | 36775 | 1244 | 29.56 |
| Tumor Mucosa 3 | 500 ng | 15249 | 70 | 217.84 |
| Distant Mucosa 1 | 500 ng | 27106 | 88 | 308.02 |
| Distant Mucosa 2 | 500 ng | 20882 | 135 | 154.68 |
| Distant Mucosa 3 | 500 ng | 12359 | 191 | 64.71 |
| WT Mucosa 1 | 300 ng | 138109 | 1299 | 106.32 |
| WT Mucosa 2 | 300 ng | 121488 | 25145 | 4.83 |
| WT Mucosa 3 | 300 ng | 4404 | 1392 | 3.16 |
| dLN 1 | 500 ng | 74528 | 26892 | 2.77 |
| dLN 2 | 500 ng | 62460 | 28414 | 2.2 |
| dLN 3 | 500 ng | 39266 | 1781 | 22.05 |
| Spleen 1 | 500 ng | 61992 | 27290 | 2.27 |
| Spleen 2 | 500 ng | 64709 | 28596 | 2.26 |
| Spleen 3 | 500 ng | 75714 | 28323 | 2.67 |

To track the clonal origins of these T cells, the similarity of TCR clonotypes within the mucosal tissue compartments was compared among different mice. Using the Jaccard index, the ratio of clonotypes overlapping between two specific compartments was measured. When the mammary gland mucosa was compared among different animals, more T cell clonotypes in the tumor-adjacent mucosa were shared among different tumor-bearing mice than among individual tumor-naïve mice. This clonotype sharing was more evident for T cells in the distant mammary gland tissues of tumor-bearing mice (FIG. 1E). Considering that only select clonotypes reside within the mammary gland mucosa, this heightened TCR similarity among tumor-bearing mice indicated that some common antigens produced by 4T1 tumors may play a role in this selection.

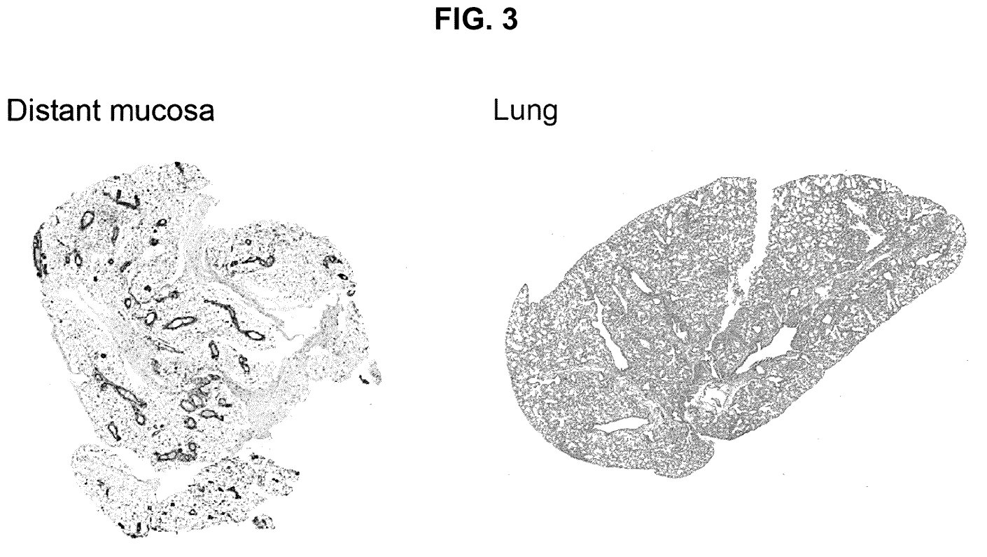

A global T cell similarity analysis between different types of mucosal tissues was also performed. Using the Morisita index, both clonotype amounts and abundance were taken into account (Venturi, V., et al. (2008) Journal of Immunological Methods 329, 67-80). Little repertoire similarity between T cells within the tumor-naïve and tumor-adjacent mucosa or between T cells from the tumor-naïve and tumor-distant mucosa was found. However, the overall similarity between T cells in the tumor-adjacent and distant mucosa from tumor-bearing mice was quite high (FIG. 1F). Since the lung is the primary site of 4T1 spontaneous metastasis, the pre-metastatic lung mucosa tissue was harvested at 3 weeks post tumor inoculation (FIG. 3) as well as the later-developed lung metastatic (secondary tumors) for TCR repertoire analysis. Clonotypes were overlapped between the primary tumor in the breast and metastatic tumors in the lung. The clonotype sharing-especially for the dominant clones favored by the Morisita index-between the primary tumor and pre-metastatic lung was as high as the sharing between the primary and secondary tumors. (FIG. 1G). This correlated with the similarity findings between the primary tumor and the distant mammary gland. In addition, the similarity between the pre-metastatic lung and the primary tumor was higher than that between the pre-metastatic lung and the lung metastases. This indicated that either there were new infiltrates after the establishment of metastasis, or there were T cell clonotypes that expand or contract, resulting in the change of dominant T cell clones. Furthermore, the overall sharing of clonotypes (measured by Bhattacharyya's Coefficient after normalization) between the pre-metastatic lung tissues and distant mammary gland mucosa was significantly elevated compared to the sharing between the pre-metastatic lung and either the mammary gland mucosa or lung tissues from tumor naïve mice (FIG. 1H). These data indicated that before the establishment of metastases, the same expanded T cell clones egressed the primary tumor and infiltrated the distant mammary gland and pre-metastatic lung tissue.

Zooming into the most abundant (top 10) clonotypes in the distant mucosa, each individual clone could be identified in the tumor-adjacent mucosa and 6 out of 10 were highly expanded (>0.5%) clones (FIG. 1I and FIG. 1J) (Jia, Q., et al. (2015) Oncoimmunology 4, e1001230). In addition, 7 out of 10 of these clones were also identified within the 4T1 tumor (FIG. 1I and FIG. 1J). Notably, these TCRs-except one-were not found in the mucosa of tumor naive mice. Taken together, this deep sequencing-aided clonal lineage tracing indicated that resident T cells found in the tumor adjacent and distant mucosa share common precursors, which most likely originated from T cells in the tumor.

### Example 2

### Single-Cell RNA-Sequencing Dissected Intra-Tumoral TEff/EM Heterogeneity

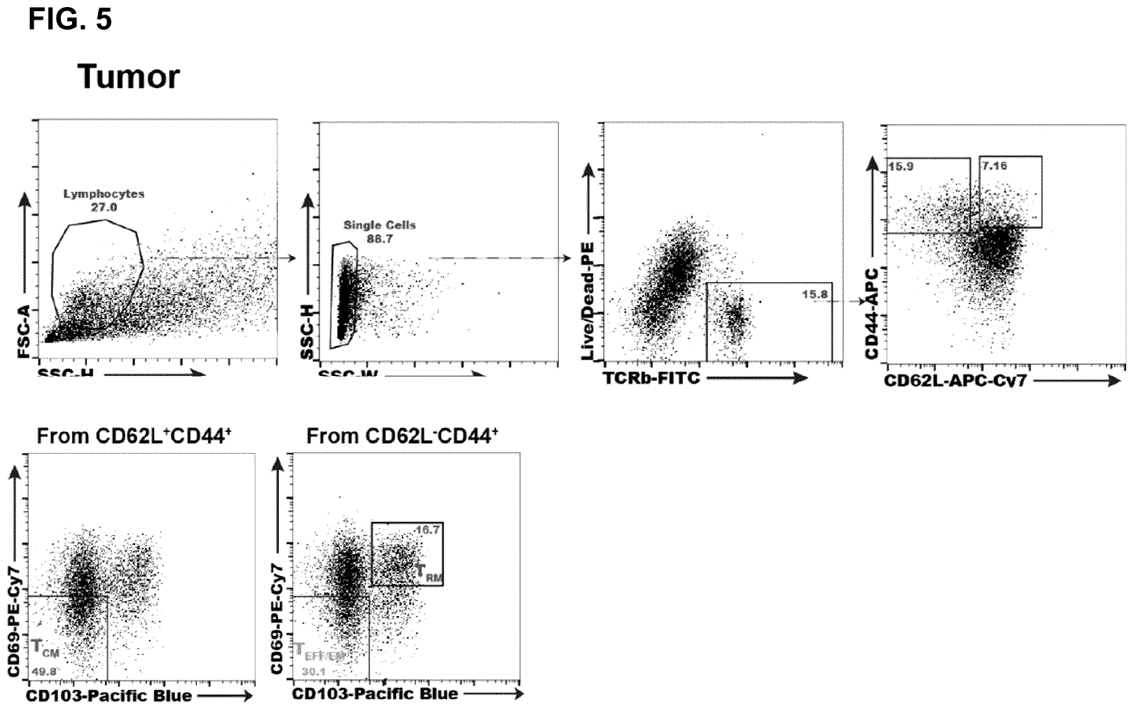

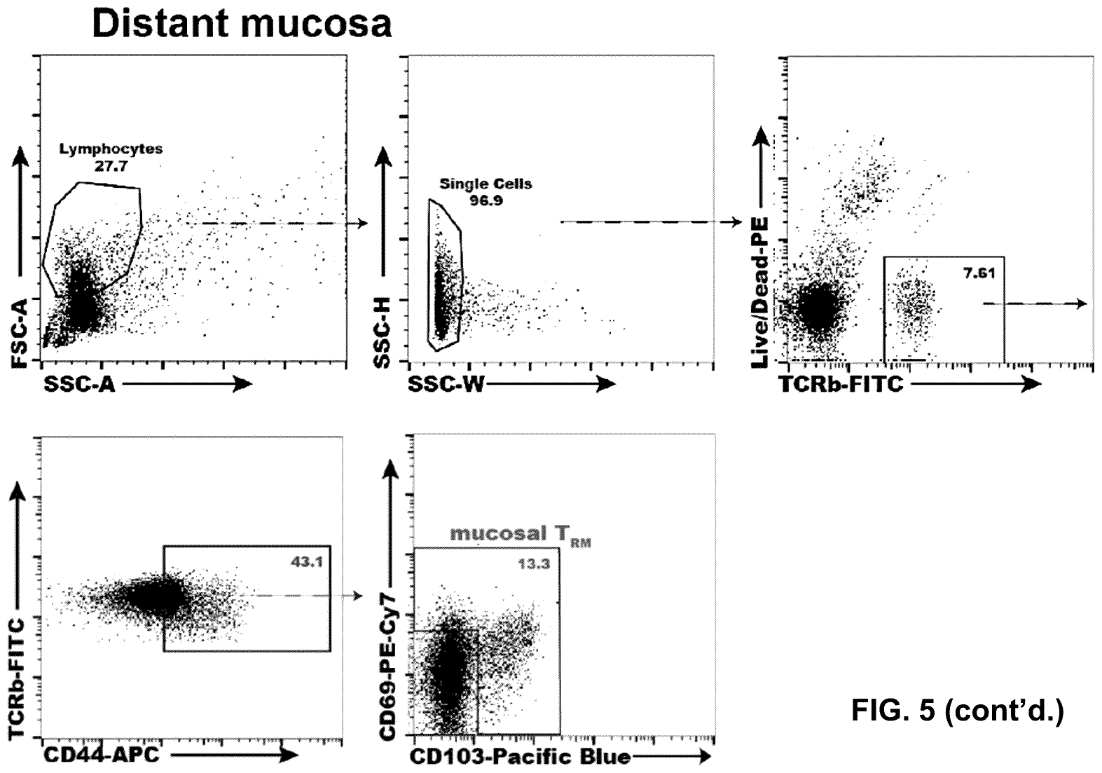

While the heterogeneity of CD103⁺ T_{RM}s is widely described, the characteristics of T_{RM} subpopulations within the tumor or distant tissues of tumor-bearing mice are less defined. To characterize tumoral T_{RM}s, single-cell RNA sequencing (scRNA-seq) technology provided unprecedented analytical power and allowed the dissection of cells with thousands of dimensions. With the hypothesis that particular intratumoral T cells are precursors for T_{RM} development, the scRNA-seq approach was expanded to include various effector and memory T cell populations so as to comprehensively depict their transcriptomic program. From the tumor and distant mucosa of mice bearing established 4T1 tumors, distinct memory T cell populations were flow sorted using a strict gating strategy (FIG. 5) (Buenrostro, J. D., et al. (2018) Cell 173, 1535-1548.e1516). Single-cell CDNA libraries were constructed from paired intratumoral sorted TCRβ⁺CD44⁺CD62L⁻CD69⁺CD103⁺ cells (T_{RM}s), mucosal sorted TCRβ⁺CD44⁺CD69⁺CD103⁺ cells (distant mucosa T_{RM}s), intratumoral TCRβ⁺CD44⁺CD62L⁺CD69-CD103″cells (T_{CM}s), and TCRβ⁺CD44⁺CD62L ⁻CD69⁻CD103 cells (T_{EM}s) (Table 2).

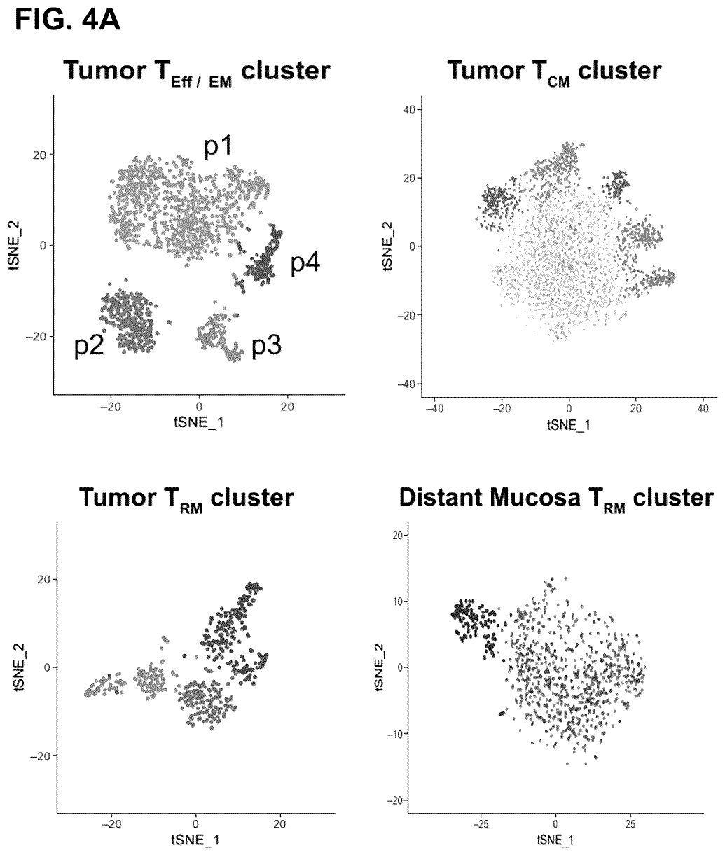

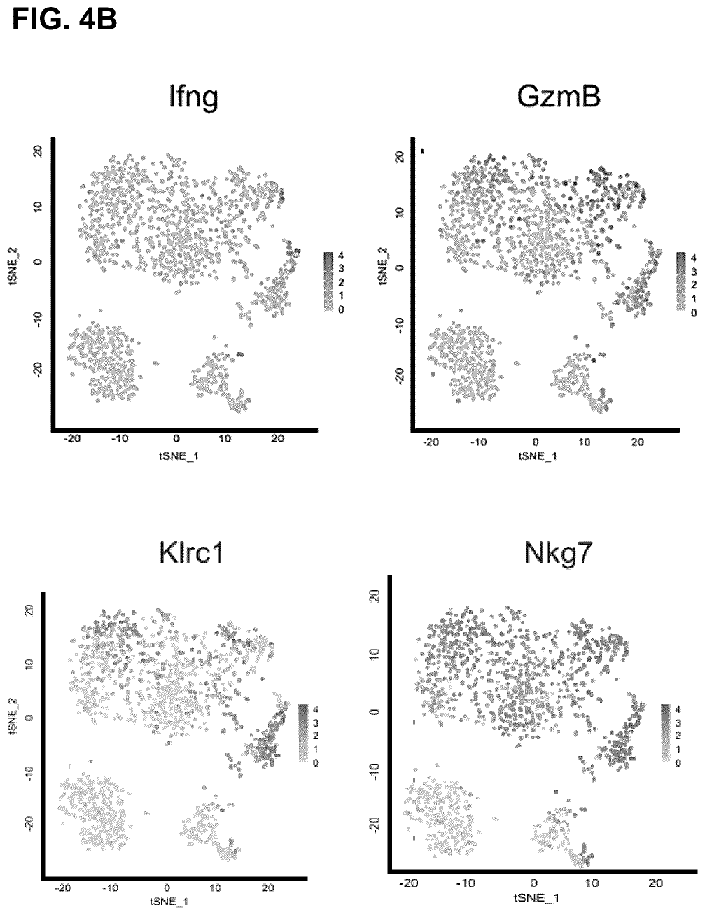

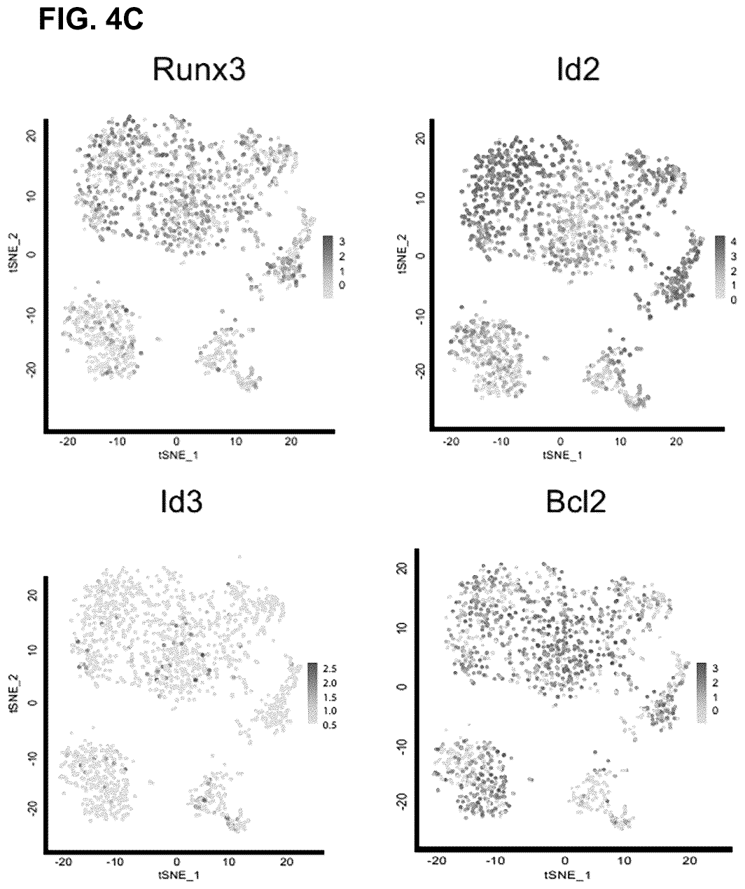

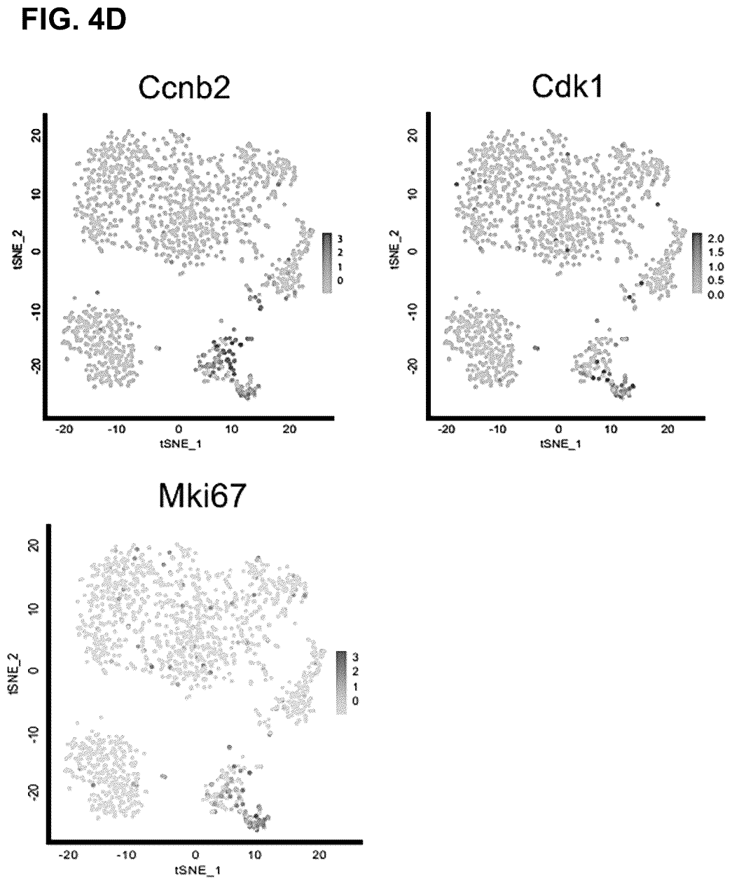

TABLE 2
Sequencing Information for Samples Submitted
for scRNA-seq Related to FIG. 4A-FIG. 4D.

| | | Mean | | | |
|---|---|---|---|---|---|
| | No. | Reads/ | Median | | Valid |
| Sample ID | Cells | Cell | Genes/Cell | No. Reads | Barcodes |
| Tumor T_{RM} | 2273 | 473741 | 1228 | 1076814147 | 0.969 |
| Tumor T_{Eff}/ | 6859 | 122127 | 1060 | 837674652 | 0.976 |
| T_{EM} | | | | | |
| Tumor T_{CM} | 9424 | 77281 | 1127 | 728297585 | 0.977 |
| Distant | 3679 | 50324 | 1155 | 185144103 | 0.971 |
| Mucosa T_{RM} | | | | | |

| | | Q30 | Reads | | |
|---|---|---|---|---|---|
| | | Bases | Mapped | Total | Median |
| | Sequencing | in | to | Genes | UMI |
| Sample ID | Saturation | UMI | Genome | Detected | Counts/Cell |
| Tumor T_{RM} | 0.971 | 0.959 | 0.864 | 14361 | 2915 |
| Tumor T_{Eff}/ | 0.935 | 0.96 | 0.899 | 15152 | 2877 |
| T_{EM} | | | | | |
| Tumor T_{CM} | 0.883 | 0.96 | 0.9 | 15529 | 3659 |
| Distant | 0.845 | 0.979 | 0.944 | 14616 | 3271 |
| Mucosa T_{RM} | | | | | |

After data processing and normalization, unbiased clustering was performed and the CD8+clusters was visualized using t-distributed stochastic neighbor embedding (t-SNE, (FIG. 4A) (Kobak, D., et al. (2019) Nature Communications 10, 5416). Through this unsupervised analysis, all four samples could be distinctly separated into multiple clusters, confirming continued heterogeneity among the single cell subsets.

In comparison to TCMs, intratumoral T_{Eff/EMs} were a more heterogeneous population, which could largely be divided into four subsets, p1-p4. The effector molecules Ifng and GzmB were highly expressed in p4, had heterogeneous expression in p1 and p3, but were largely absent from p2. Similar expression patterns were applied to well-known effector surface markers for cytolytic T cells such as Klrc1 and Nkg7 (FIG. 4B). From this, p4 was reasoned to be highly enriched by effector cells. All of the four subsets highly expressed the transcription factor Runx3, which is essential for the cytotoxic program (Cruz-Guilloty, et al. (2009) J Exp Med 206, 51-59) as well as Id2, which is upregulated to support the effector phase of cytotoxic T lymphocytes (CTL) (Cannarile, M. A., et al. (2006) Nat Immunol 7, 1317-1325) responses. Comparing p2 to p4, the density of Runx3- and Id2-expressing cells was slightly lower in p2. This was accompanied by an opposite pattern of Id3 (which is a transcription factor whose expression is crucial for effector memory development (Yang, C. Y., et al. (2011) Nat Immunol 12, 1221-1229)) which was absent in p4 (FIG. 4C). Taken together, T_{Eff/EM} p4 was enriched by differentiated effector CTLs and T cells in the p2 subset were at a more advanced TEM stage. Bcl2, a transcription factor for T cell survival in the effector and memory phases, was abundant in most sorted T_{Eff/EM} populations except p3. After assessing the cell cycle programs, p3 was determined to be a highly proliferative subset as evidenced by expression of genes restrictively expressed in the S and M phases (such as Ccnb2, Cdk1 and Mki67). (FIG. 4D). Together, the p2 subset was constituted by proliferative and less differentiated T_{Eff} cells.

### Example 3

### Tumor and Distant Mucosa T_{RM}s were Comprised of Two Distinct Populations that Resembled Either T_{EM}s or T_{CM}s

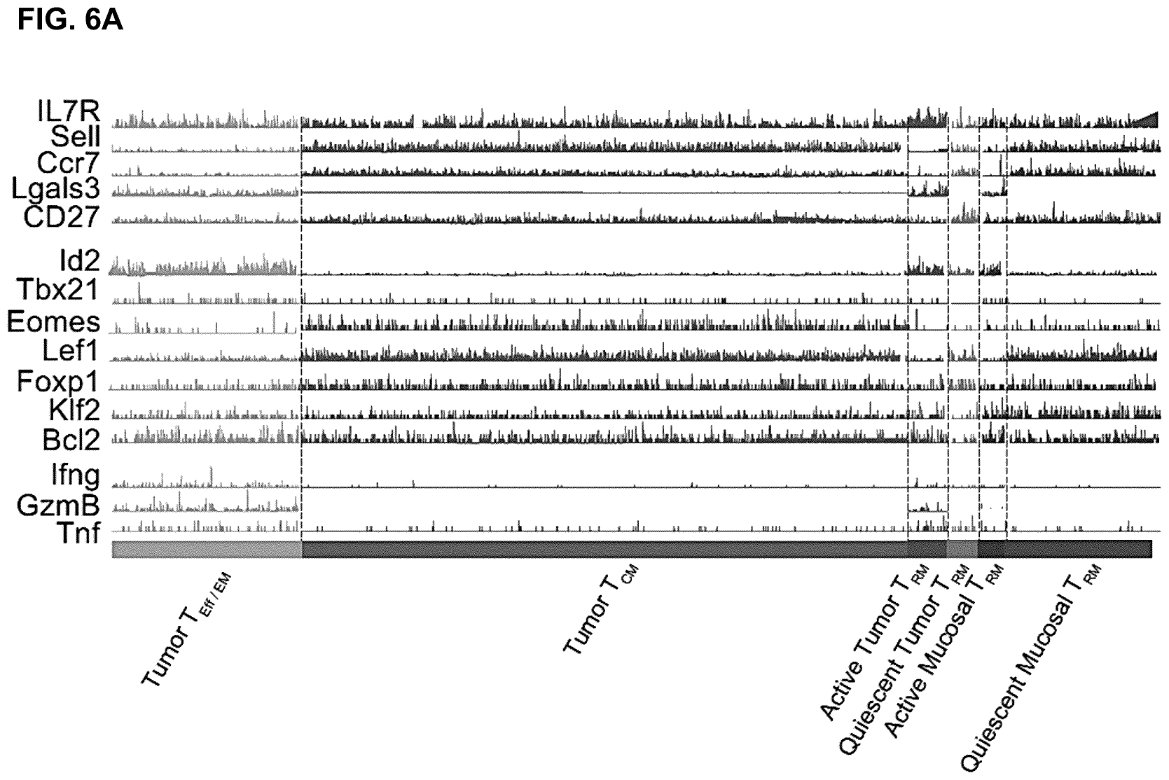

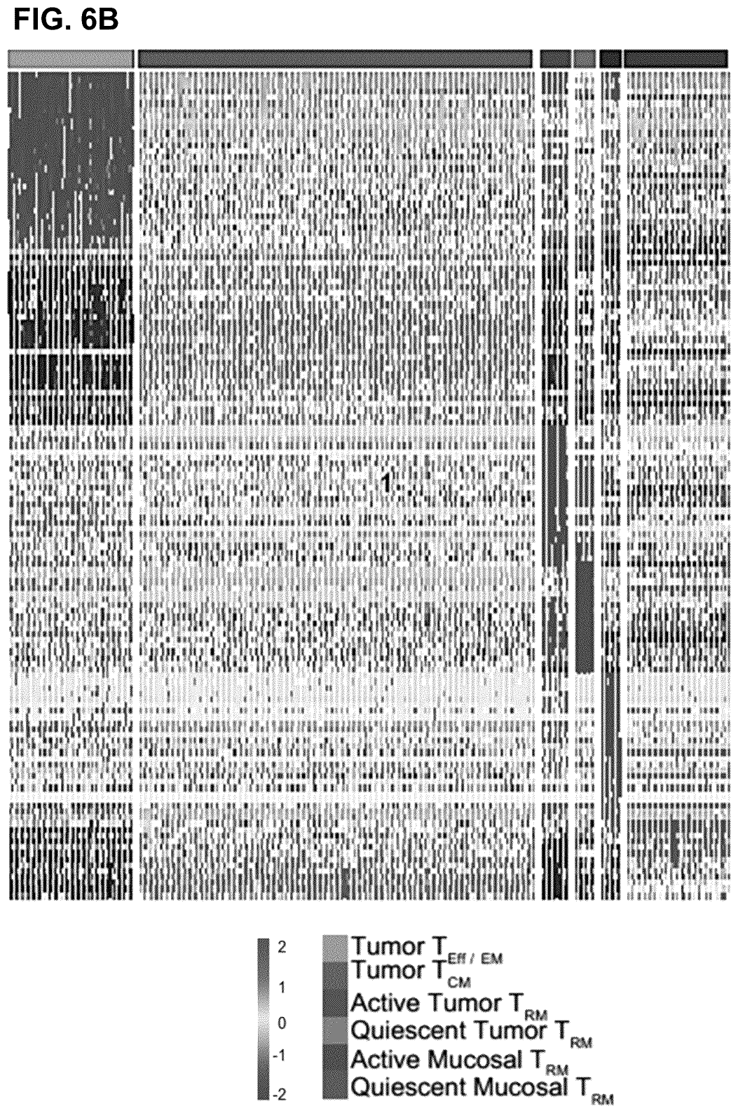

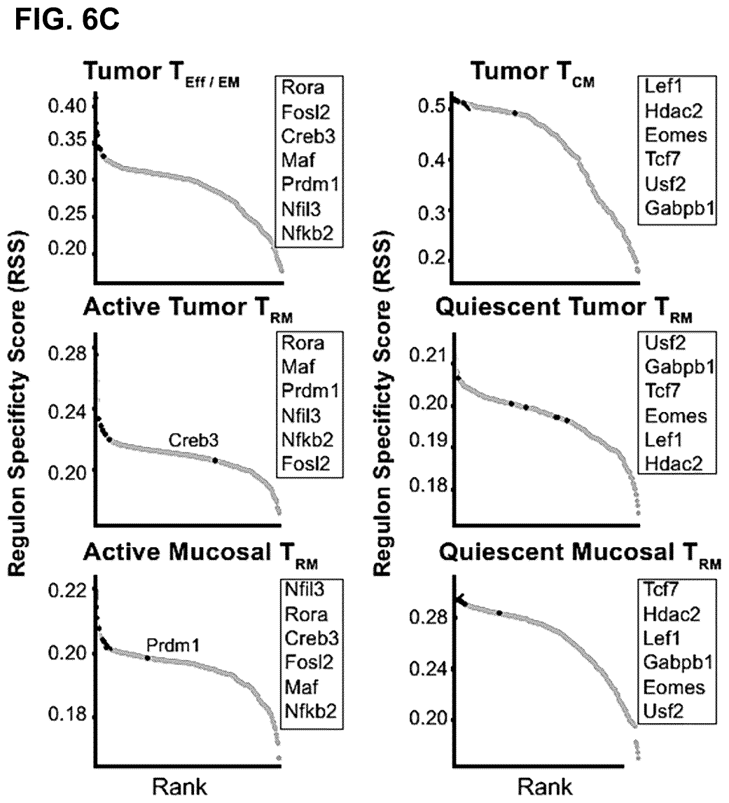

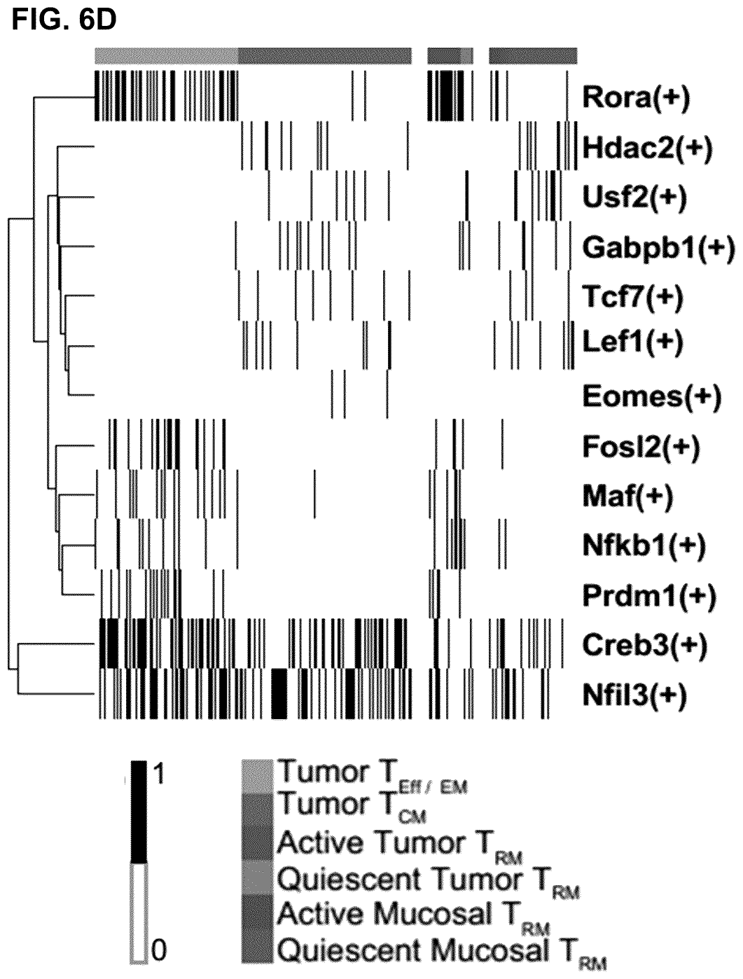

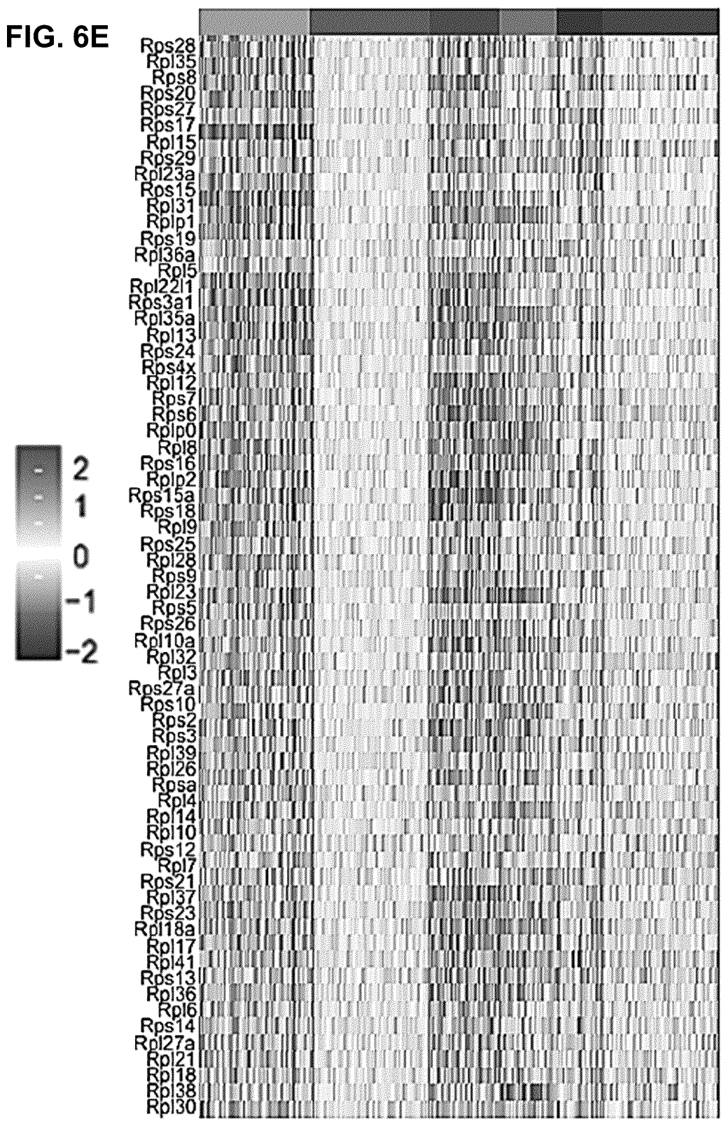

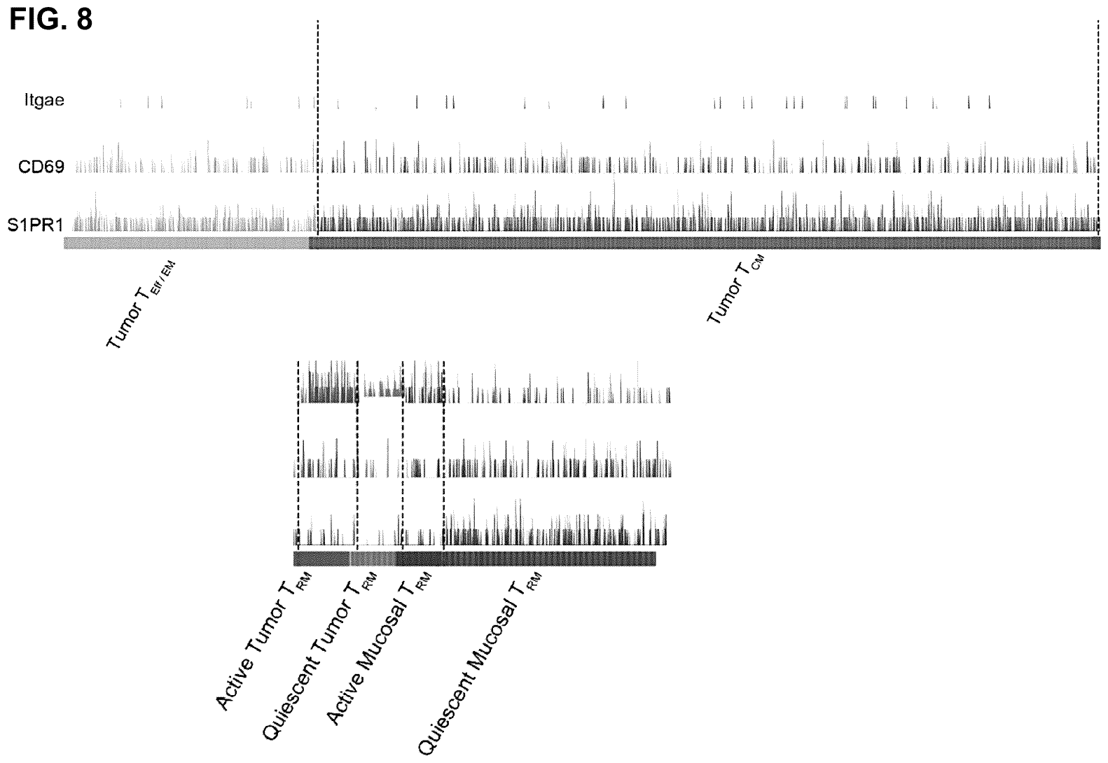

Both the tumor T_{RM} and distant mucosa T_{RM} populations independently clustered into three and two major groups, respectively. As expected, the tumor and distant mucosa T_{RM} populations showed high expression of Itgae (CD103) and low expression of S1PR1, confirming their tendency to reside within the tissue (FIG. 8). Choosing the major subsets of T_{CM}s and T_{EM}s as references (p1 of T_{Eff/EM}), these T_{RM} subpopulations were interpreted using a candidate approach by evaluating their surface markers, transcription factors, and effector molecules. A subset of T_{RM}s shared lineage characteristics with T_{EM}s, indicating that this subset of T_{RM}s was maintained at a functionally active stage-hereafter called “active T_{RM}s”. First, in active T_{RM}s, similar to their T_{Eff/EM} counterparts, Sell expression was completely suppressed (Sallusto, F., et al. (1999)) and Ccr7 expression was severely reduced. Notably, the binary expression of Lgals3 (Galectin-3) distinguished T_{EM}s from T_{CM}s and distinguished active T_{RM}s from quiescent T_{RM}s. This was consistent with previous findings that optimal Galectin-3 induction required TCR signaling (Joo, H.-G., et al. (2001) Journal of Leukocyte Biology 69, 555-564), and in the tumor microenvironment, Galectin-3 was differentially expressed on the surface of tumor-antigen activated CD8⁺ T cells, but was absent in resting T cells (Joo, H.-G., et al. (2001)). Second, similar to that in TER/EMS, Id2 (Cannarile, M. A., et al. (2006) Nat Immunol 7, 1317-1325) was highly elevated in the active T_{RM} subsets. Of note, since the CD8⁺ T cells were memory-phenotype enriched, transcription factors that preferentially support early effector function such as Tbx21 (T-bet) (Intlekofer, A. M., et al. (2005) Nat Immunol 6, 1236-1244) were detected occasionally. Nevertheless, more frequent Tbx21 expression was observed in active T_{RM}s than quiescent T_{RM}s. Third, similar to T_{Eff/EMS}, Ifng and GzmB were preferentially expressed in active tumor T_{RM}s (FIG. 6A).

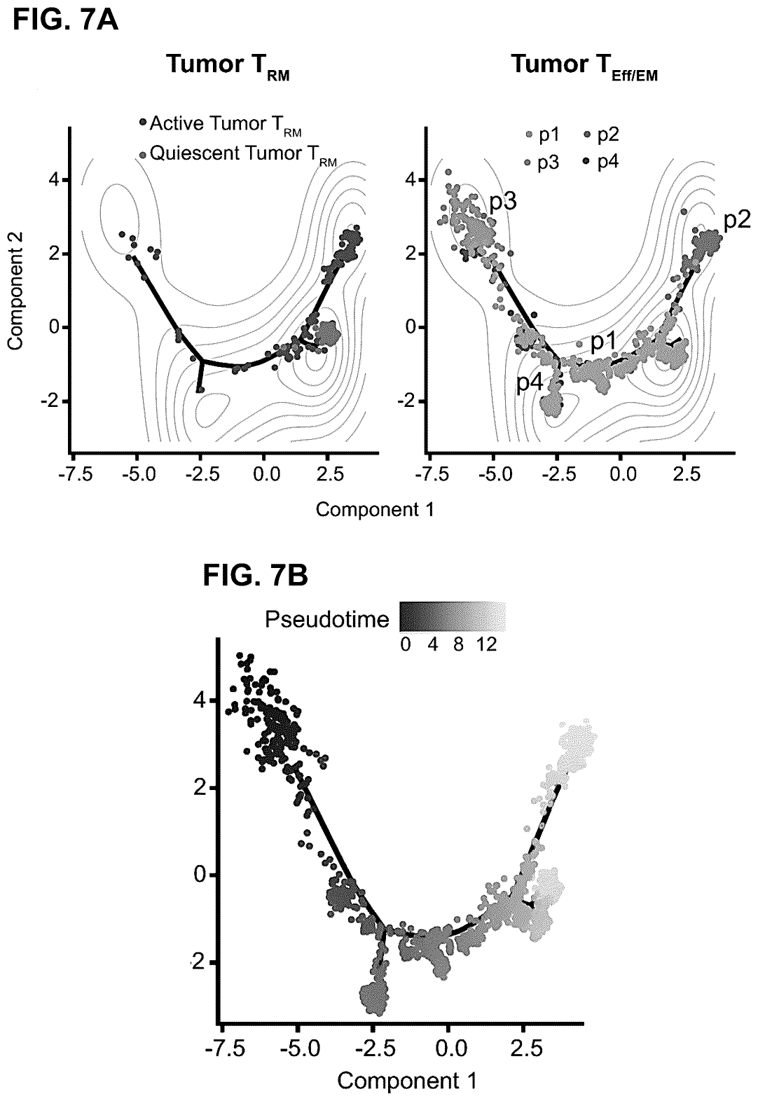

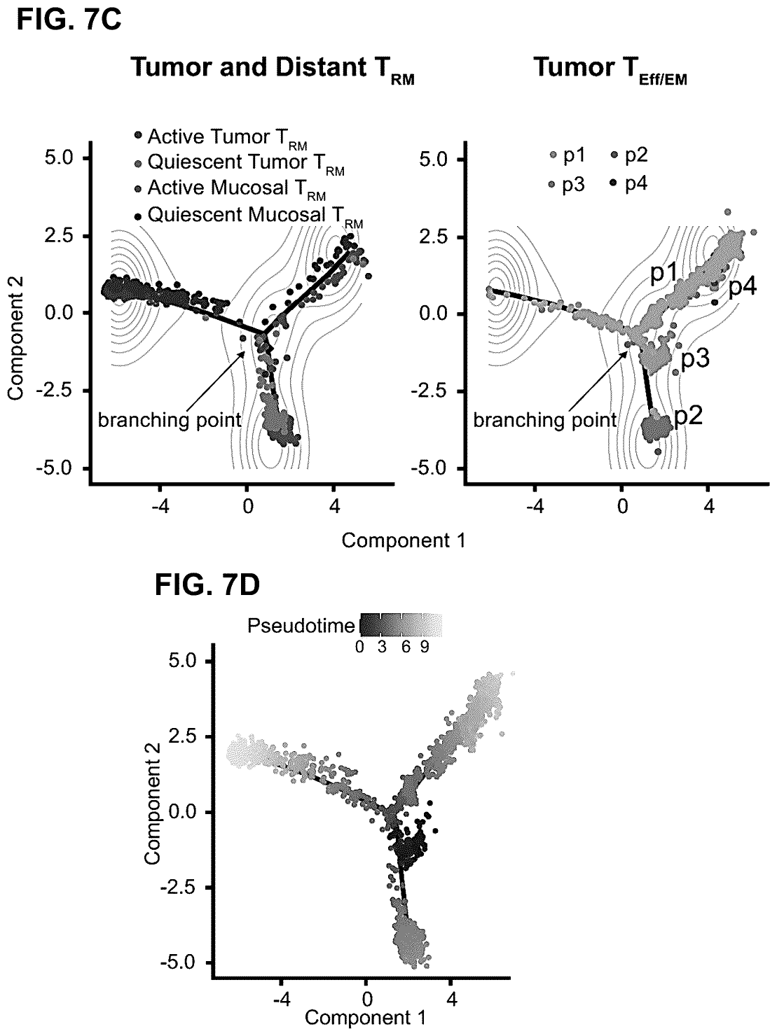

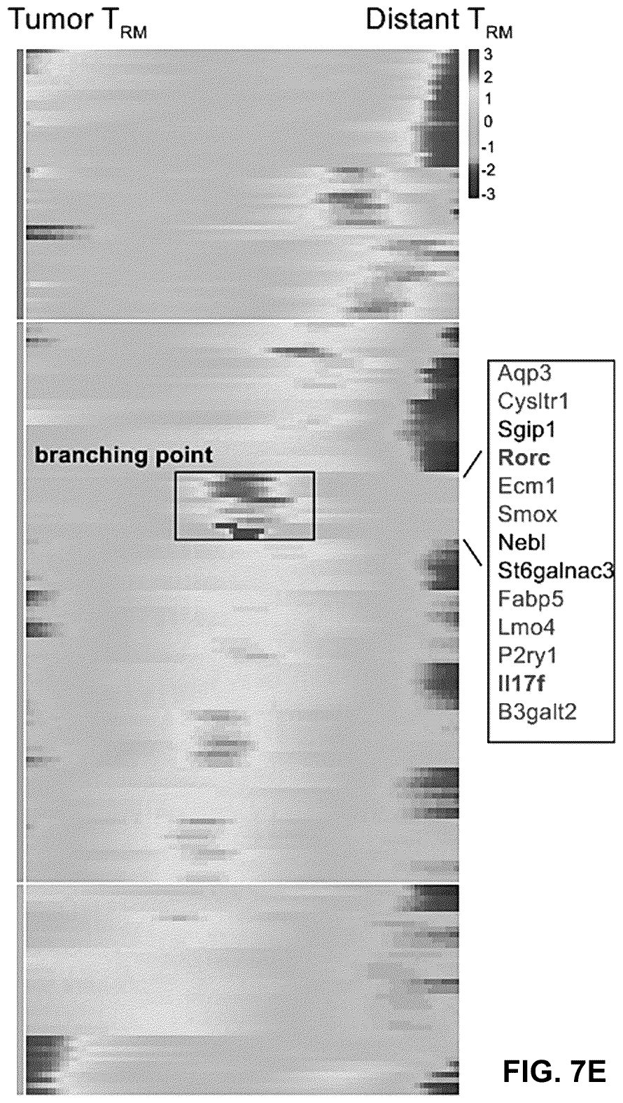

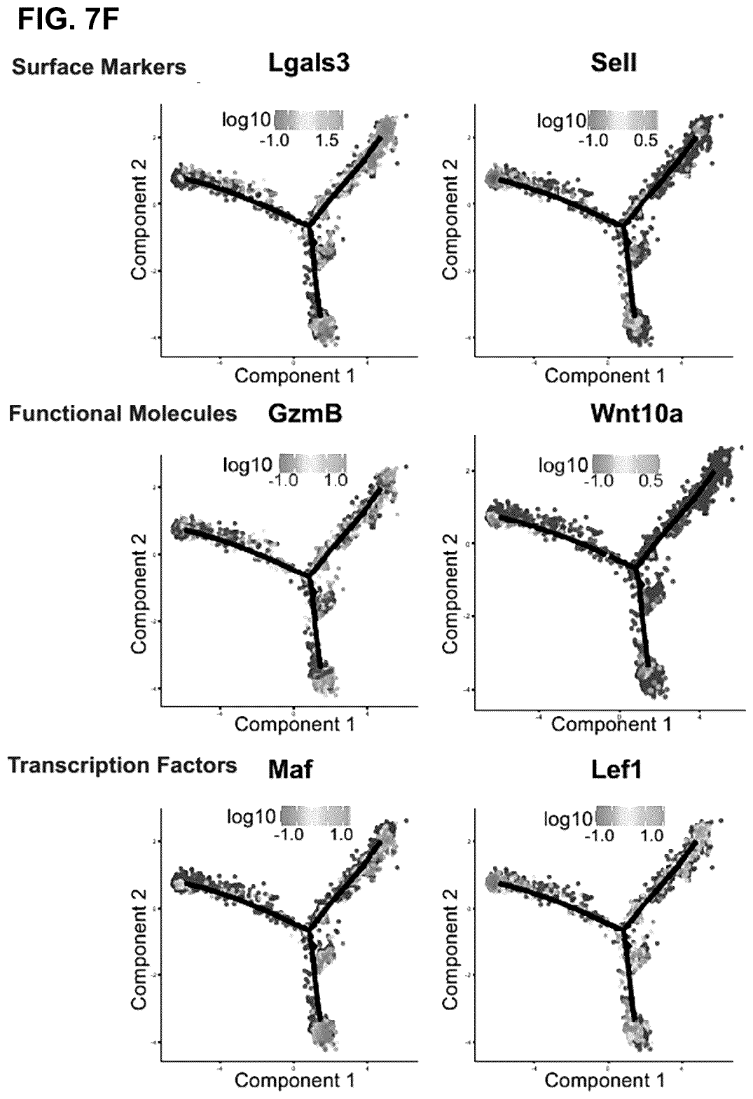

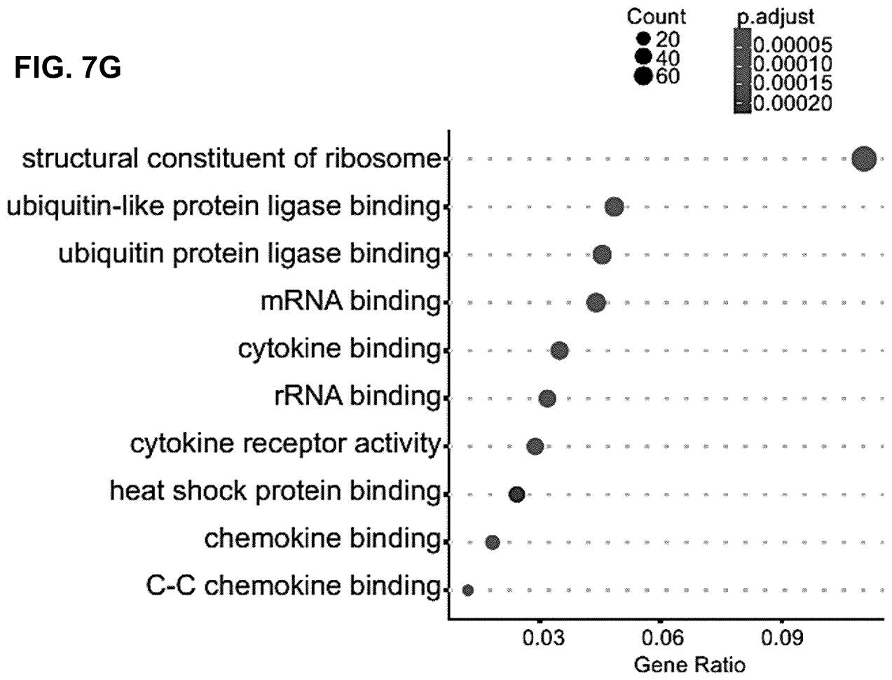

Reciprocally, within both the tumor and distant mammary gland mucosal compartments, another subset of T_{RM}s was found to have gene expression features resembling T_{CM}s, representing a quiescent, long-lived resident memory T cell population-hereafter called “quiescent T_{RM}s.” Quiescent T_{RM}s were found in both the tumor and distant mucosa compartments. Specifically, in quiescent T_{RM}s, the following observations were made. First, the T_{CM} surface markers Sell (CD62L) and CCR7 were highly expressed. While CD27 was widely expressed in all selected subsets, its expression was elevated in the quiescent subsets, which indicated its naïve-like feature (van Lier, et al. (1987) J Immunol 139, 1589-1596). Second, T_{CM}-associated transcription factors such as Eomes (Pearce, E. L., et al. (2003) Science 302, 1041-1043), Lef1 (Zhou, X., et al. (2012) Journal of Immunology 189, 2722-2726) and Foxp1 (Feng, X., et al. (2010) Blood 115, 510-518) were highly expressed in the quiescent T_{RM} population. Third, like in T_{CM}s, the expression of functional molecules associated with cytolytic killing, such as Ifng, GzmB and Tnf were silenced. (FIG. 7A). Taken together, T_{RM}s, both in the tumor and mammary gland mucosal compartments, were largely divided into two functional phenotypes based on their signature gene expression.

The T_{Eff/EM} versus T_{CM} phenotypic division was also validated by global differences of their transcriptomes. The expression levels of the top 100 differentially expressed genes (DEGs) in the intratumoral T_{EM} and T_{CM} populations were used as a benchmark. When this unbiased control was applied to different T_{RM} subsets, it demonstrated the major differences between active and quiescent T_{RM}s (FIG. 6B).

To examine whether these transcriptomic differences represented a globally orchestrated differentiation event, a transcriptomic regulon analysis was performed. The SCENIC (single-cell regulatory network inference and clustering) algorithm was employed to identify co-expressed gene modules that shared common cis-regulatory elements for a specific transcription factor (Aibar, S., et al. (2017) Nat Methods 14, 1083-1086). Based on p-values that reflected the co-expression coefficients and enrichment of cis-regulatory elements, the transcription factor regulons in classical T_{Eff/EMS} and T_{CM}s were ranked. Although the ranking order may vary, the majority of the top-ranked T_{Eff/EM} regulons such as Rora (Best, J. A., et al. (2013) Nat Immunol 14, 404-412), Fosl2 (Ciofani, M., et al. (2012) Cell 151, 289-303), Creb3 (Chan, C. P., et al. (2011) Cell & Bioscience 1, 6), Maf (Ciofani, M., et al. (2012)), Prdm1 (Kallies, A., et al. (2009) Immunity 31, 283-295), Nfil3 (Kashiwada, M., et al. (2011) EMBO J 30, 2071-2082), and Nfkb1 (Best, J. A., et al. (2013) Nat Immunol 14, 404-412) were also identified as top ranked regulons for active T_{RM}s in the tumor and distant mammary mucosa. Reciprocally, all top-ranked T_{CM} regulons, such as Lef1 (Zhou, X., et al. (2012)), Hdac2 (Shin, H. M., et al. (2013) Immunity 39, 661-675), Eomes (Pearce, E. L., et al. (2003) Science 302, 1041-1043), Tcf7 (Zhou, X., et al. (2010) Immunity 33, 229-240), Usf2 (Suo, S., et al. (2018) Cell Rep 25, 1436-1445.e1433) and Gabpb1 (Luo, C. T., et al. (2017) Nature Communications 8, 1062) were significantly enriched in the transcriptome of quiescent T_{RM}s (FIG. 6C and FIG. 6D). This analysis revealed that the transcriptomic distinctions between active and quiescent T_{RM}s were controlled by the same underlying gene regulatory networks that specify T_{EM} VS. T_{CM} development.

While investigating the DEGs between these memory T cell populations, an unexpected enrichment of a large cluster of ribosome-related genes that were distinct between intra-tumor TEff/EMs and TCMs was identified. While this differential expression was less pronounced between intratumoral TRMs, expression of these ribosome genes was dramatically upregulated in the quiescent distant TRM population compared to active distant TRMs (FIG. 6E). The association between an upregulated ribosome gene profile and a relatively quiescent T cell phenotype was a common feature identified through these analyses.

### Example 4

### Tissue Environment has a Significant Role in Shaping the Transcriptome of T_{RM}s

To trace the developmental path of T_{RM}s, all tumor T_{Eff/EM} and T_{RM} cells were subjected to the Monocle2 algorithm for pseudotime analysis (Qiu, X., et al. (2017) Nat Methods 14, 979-982; Trapnell, C., et al. (2014) Nature biotechnology 32, 381-386). The assumption was that within the tumor, T cells differentiate or develop asynchronously. The moment of sample collection represented a snapshot, where each individual T cell was fixed at a specific stage of differentiation or development. Consequently, the differentiation and developmental processes of the T cells was revealed by arranging individual cells on a time trajectory based on their gradual and continuous transcriptomic transition. Hence, pseudotime analysis allowed for the determination of the relative position of each individual cell on this time trajectory. Since the relative positioning of cells cannot automatically determine the beginning or end of the pseudotime, the least differentiated T_{Eff/EM} population with the highest proliferative capacity (T_{Eff/EM} p3, FIG. 4D) was designated as the starting point. This was a reasonable assumption because T cell proliferation in the effector phase proceeded terminal effector and memory T cell differentiation, including the development of T_{RM}s. In the pseudotime plot, cells found in the heterogeneous p1 subset directly connected to the p3 population, which spread widely on the development tree. This “synthetic” developmental process had two branched ends: (i) one was a tight cluster encompassing a majority of quiescent T_{RM}s; and (ii) the other was enriched by highly differentiated p2 T_{EM}s and active T_{RM}s (FIG. 7A and FIG. 7B). This indicated that by stemming from T_{Eff}s (p3), T_{EM}s and T_{RM}s shared a similar development process until they reach their final branching point.

In both the transcriptome and regulon analyses, the distinction between quiescent and active T_{RM}s in the tumor was less profound than that in the distant mammary gland mucosa (FIG. 6B, FIG. 6C, and FIG. 6D). This blurred distinction was also reflected by the differential expression of ribosome-related genes (FIG. 6E), indicating that quiescent T_{RM}s in the tumor were relatively “activated” in comparison to their counterparts in the distant mammary gland. To test the impact of different tissue microenvironments, as well as to explore the lineage progression paths leading to distant T_{RM} development, T_{RM}s collected from the distant mammary gland were added to the Monocle2 algorithm. Choosing the same proliferating p3 subset as the starting point, three major observations FIG. 7C-FIG. 7D) were made. First, except for some active distant T_{RM}s that co-localized with active tumor T_{RM}s, the majority of T_{Eff/EM} and T_{RM} cells were separated by their tissue of origin with the mucosal T_{RM} subsets occupying a different space on the contour plot from the tumor memory T cell subsets. It was unexpected to observe that the impact of the tumor or distant mucosa location could overshadow the intrinsic transcriptomic differences between T_{Eff/EMS} and T_{RM}s. Second, a few tumor T_{Eff/EM} p1 cells crossed the tissue “boundary” to overlap with distant mucosa T_{RM}s. This revealed that there are T_{Eff/EM} cells with a transcriptome that mimics distant T_{RM}s, although their surfaces are absent of CD103 expression and they are still located in the tumor. Third, a transitioning population of tumor T_{Eff/EM} cells was located at a critical branching point. These precursor T_{Eff/EM} cells largely belonged to p1 and p4 subsets and leave this branching point with lineage decisions to become tumor T_{RM}s or distant T_{RM}s.

The transcriptomic transition of individual T cell subsets approaching and leaving this branching were projected onto a heat map, which allowed for the identification of signature gene features that comprise this branching point. (FIG. 7E). A cluster of 13 genes was identified and 11 out of 13 of these genes were signature genes identified in the Th17 lineage, including the effector molecule Il17f and the master transcription factor Rorc (Chang, S. H., et al. (2009) Cytokine 46, 7-11; Ciofani, M., et al. (2012) Cell 151, 289-303; Gobert, A. P., et al. (2018) Front Immunol 9, 1242-1242; Hu, R., Huffaker, et al. (2013) Journal of Immunology (Baltimore, Md: 1950) 190, 5972-5980; Kim, H. S., et al. (2015) Journal of Immunology 194, 185.114-185.114; Li, B., et al. (2009) J Immunol 182, 7625-7633; Skepner, J., et al. (2014) Journal of Immunology 192, 2564-2575; Su, P., et al. (2016) J Immunol 197, 1054-1064; Tu, Z., et al. (2018) Journal of Investigative Dermatology 138, 1078-1087; Zhou, X., et al. (2016) Journal of Immunology 196, 186.116). These Tc17-like cells can serve as intermediate progenitors to separate T_{RM}s located in the tumor or distant mammary gland mucosa.

To exemplify the differences between tumor or distant T_{RM}s, phenotypic genes that specify distinct T cell stages were selected and their expression was portrayed on the pseudotime plot. Genes associated with T_{Eff/EMS} such as Lgals3, GzmB, and Maf were preferentially expressed in both TER/EMS and T_{RM}s within the tumor environment. Reciprocally, genes associated with T_{CM}s such as Sell and Lef1, or Wnt10a—which induces anabolic T cell metabolism (Terauchi, M., et al. (2009) Cell Metab 10, 229-240) and is differentially expressed in long-lived periphery T_{CM}s (Miron, M., et al. (2018) J Immunol 201, 2132-2140)—were robustly expressed in distant T_{RM}s. (FIG. 7E). Notably, when the top 200 genes differentially expressed in the tumor and distant mucosa T_{RM}s were subjected to gene ontology analysis, a large set of genes comprising the structural content of the ribosome were highly elevated in the distant T_{RM} population, which was similar to what was seen T_{CM}s. (FIG. 7G). In contrast, genes involved in cytokine and chemokine signaling were upregulated in the tumor T_{RM} population, highlighting the inflammatory nature of this population. Taken together, the transcriptional program of tumor T_{RM}s was adapted to the inflammatory nature of the tumor microenvironment. Meanwhile, in the reservoir of the tumor-free mammary mucosa, distant T_{RM}s developed into a quiescent T_{CM}-like phenotype to favor their long-term survival.

### Example 5

### CXCR6 Expression Defined a Unique Subpopulation of T_{Eff/EMS}

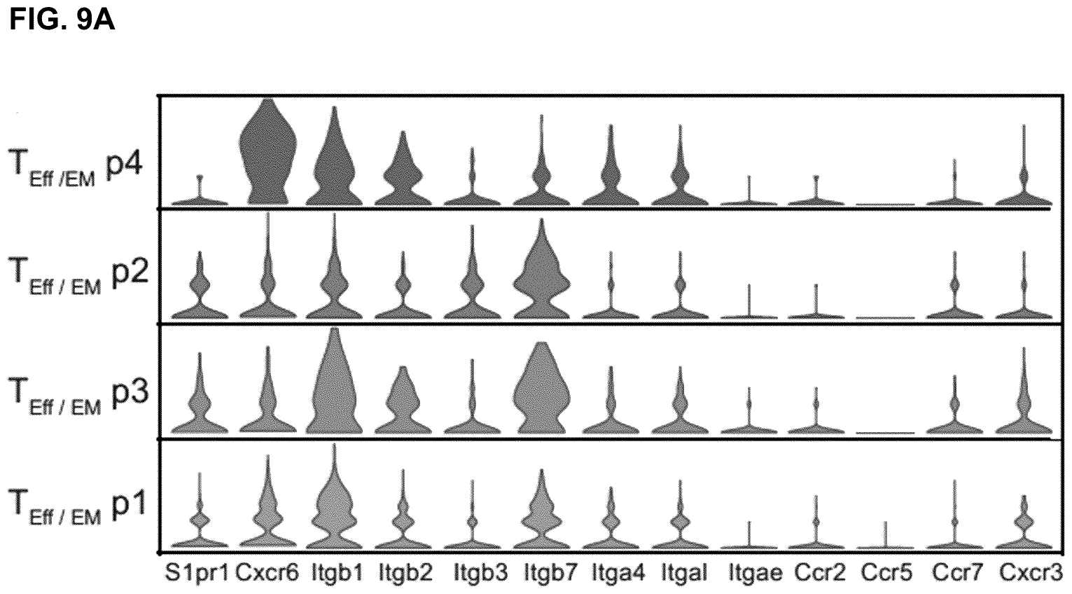

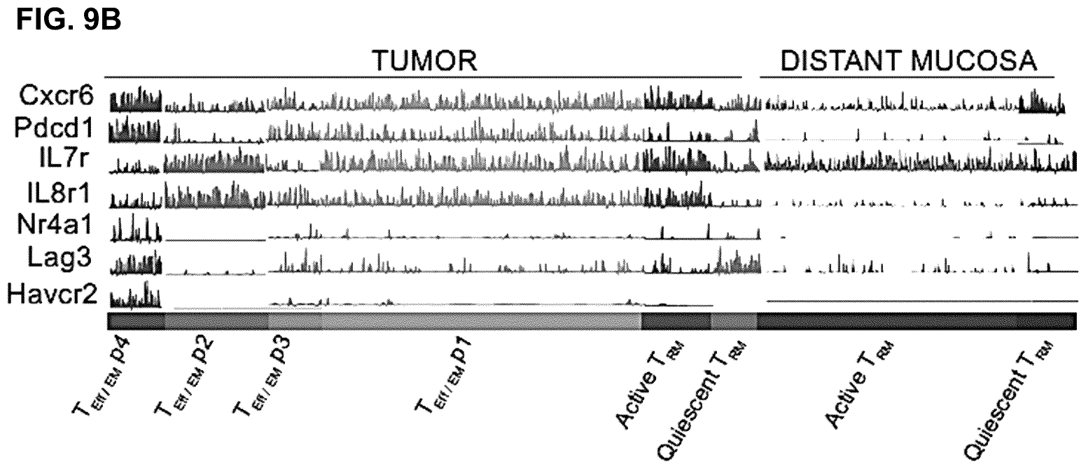

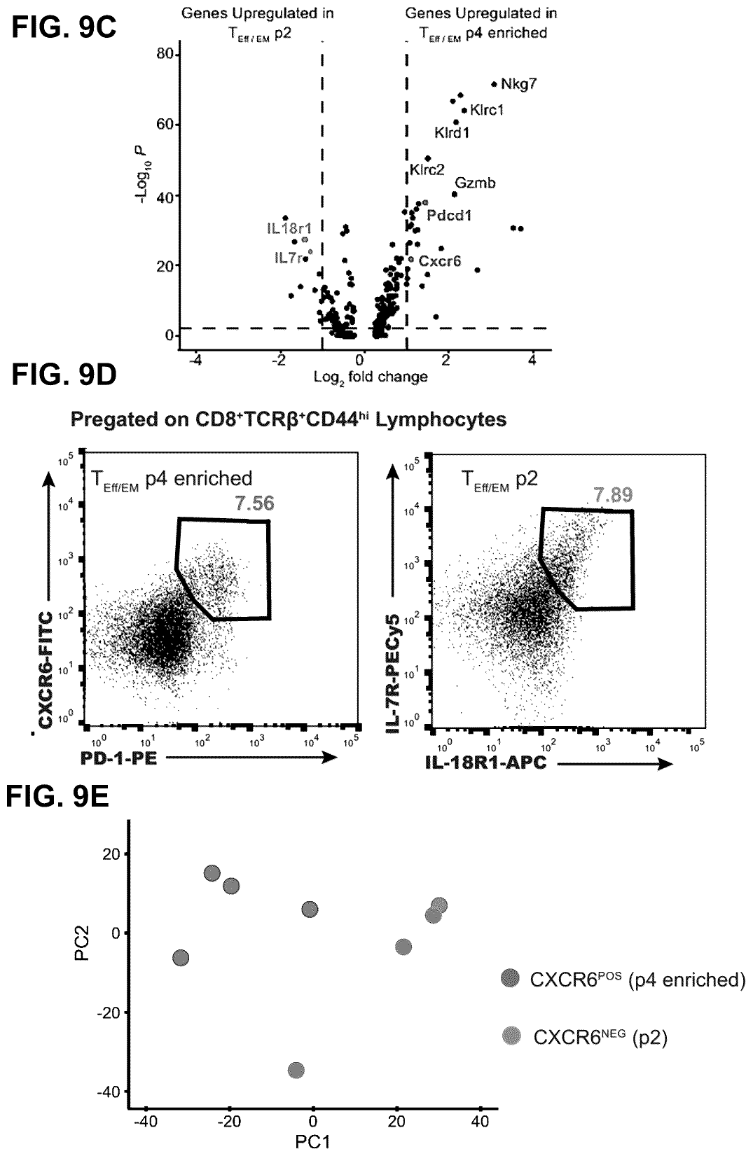

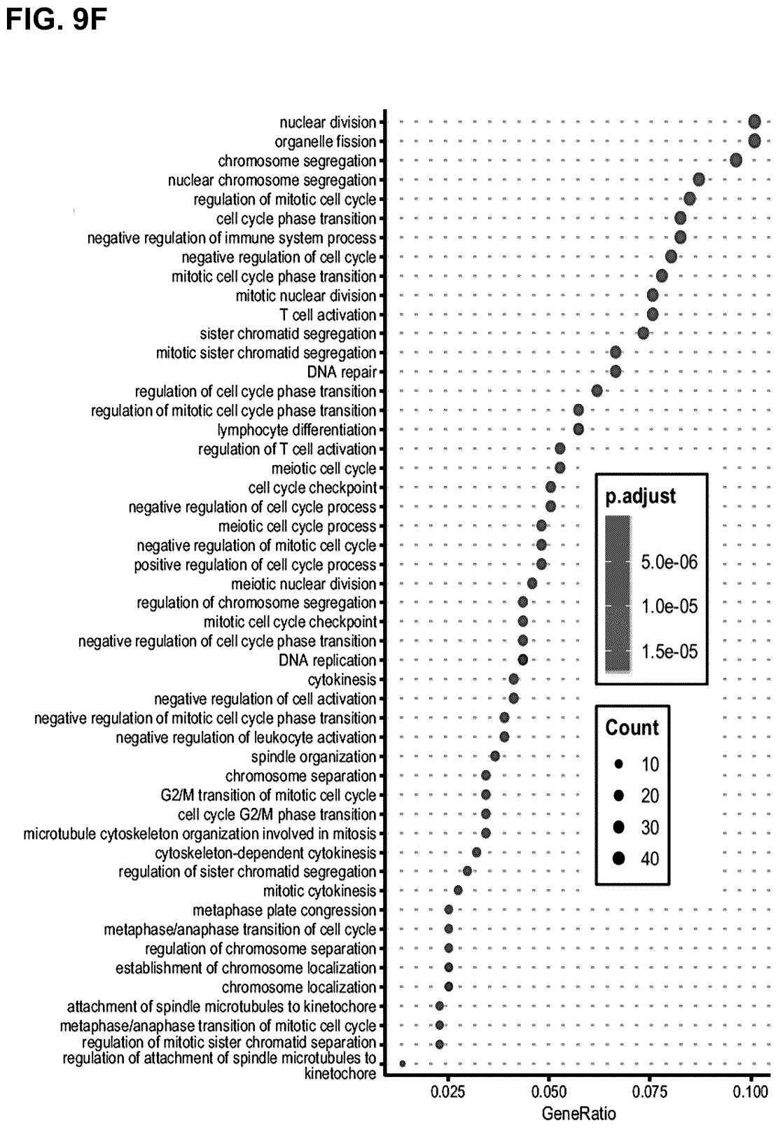

Identifying the intratumor precursor of distant T_{RM}s within the heterogeneous T_{Eff/EM} pool was next undertaken. It was reasoned that these precursor cells should have a distinct chemokine sensing and extracellular matrix rolling profile to facilitate tumor egress. A list of chemokine receptors and integrins was compiled and their expression was evaluated. S1pr1, a well-known hallmark for T cell tissue egress (Cyster, J. G., et al. (2012) Annu Rev Immunol 30, 69-94), was chosen as a benchmark. Among all T_{Eff/EM} populations, S1pr1 expression was silenced in TER/EM p4, indicating that this was a population that lacked the potential to egress. In p4, compared to other chemokine receptors, Cxcr6 was highly expressed; and when compared to the other three populations, p4 was the only population that preferentially upregulated Cxcr6. (FIG. 9A).

The elevated Cxcr6 expression in p4 was associated with enhanced Pdcd1 and reduced IL7r expression, in direct opposition to p2. (FIG. 9B). These two populations of cells were virtually sorted and their gene expression at the transcriptomic level was then compared. (FIG. 9C). The differential expression of Nkg7 and Klrc/d family members indicated that these were highly active effector T cells, evidenced by high expression of effector molecules such as Gzmb. On the contrary, for T cells in T_{Eff/EM} p2, the upregulation of IL7r was associated with IL18r1. (FIG. 9C). For tumor-infiltrating T cells isolated from non-small cell lung cancer samples, IL-18R marked a functional Tbet⁺Eomes⁺ T_{EM} population (Timperi, E., et al. (2017) Oncoimmunology 6, e1328337).

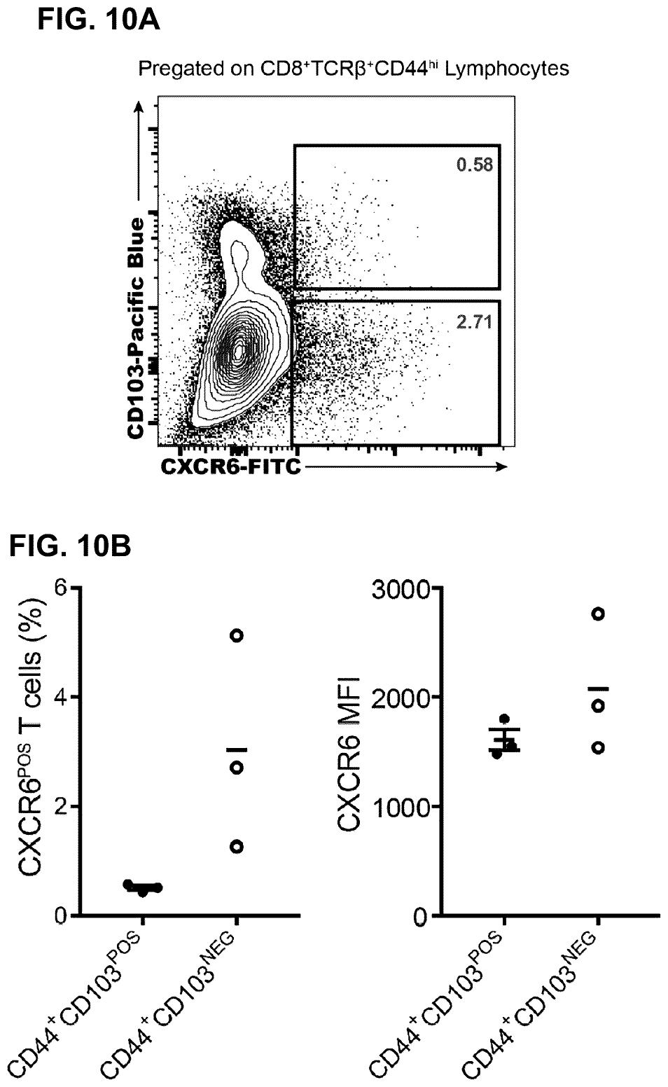

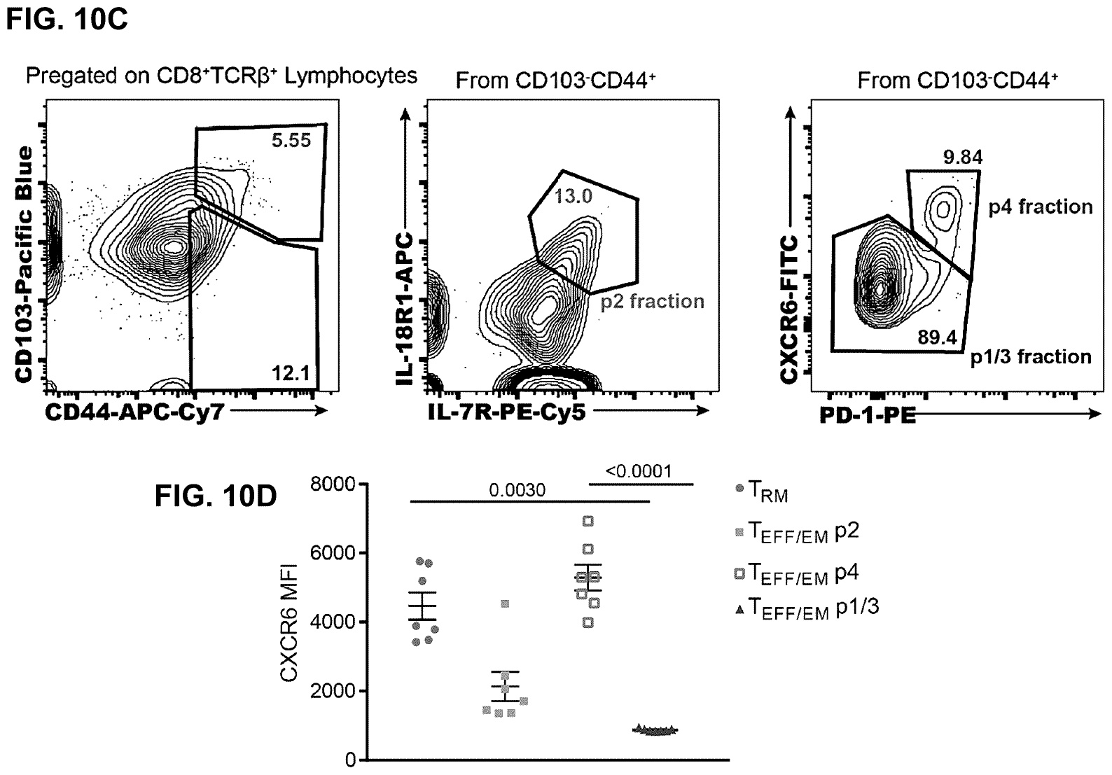

Taking the co-expression of CXCR6 and PD-1 or IL-7R and IL-18R1 as new stage-specific markers, using mice with established 4T1 tumors, CD44^{hi}CD103⁻CXCR6⁺PD-1⁺T_{Eff/EM} cells, which were highly enriched with the p4 subset (described above), and CD103⁻IL7R⁺IL18R1⁺TER/EM cells, which comprised the p1 and p2 subsets, were sorted (FIG. 9D) to validate the findings through RNA-seq. Bulk RNA-seq analysis validated that these two populations have distinct transcriptomic profiles. (FIG. 9E). The levels of CXCR6⁺on tumor T_{RM}s were compared to that of T_{Eff/EMS} and it was found that overall, more T_{Eff/EM} cells expressed CXCR6⁺ (FIG. 10A-FIG. 10B). Flow cytometry analysis validated that CXCR6 expressing T_{Eff/EM} cells were enriched in p4. Within this subpopulation, at the individual cell level, the surface expression of CXCR6 was indistinguishable from that of T_{RM}s. (FIG. 10C and FIG. 10D) (Takamura, S., et al. (2019) Journal of Experimental Medicine, jem.20190557; Wein, A. N., et al. (2018) Journal of Experimental Medicine, jem.20181308). Gene ontology pathway analysis overwhelmingly showed that the CXCR6⁺subset of T_{Eff/EMS} was actively in cell cycle, indicating that this population was proliferative. (FIG. 9F). This analysis illustrated that these CXCR6⁺ effector cells were quite unique. On one hand, these CXCR6⁺ effector cells could be labelled as terminally exhausted cells (Wherry, E. J., et al. (2007) Immunity 27, 670-684) based on their elevated expression of Pdcd1, Nr4a1 (Liu, X., et al. (2019) Nature 567, 525-529), Lag3 and Havcr 2 (Tim-3). While, on the other hand, the transcriptional program in these CXCR6⁺ effector cells overrode all these possible inhibitions and maintained these cells in cell cycle with a robust cytolytic program.

### Example 6

### CXCR6-T_{Eff/EMs} Were Precursors for Distant TRM Formation

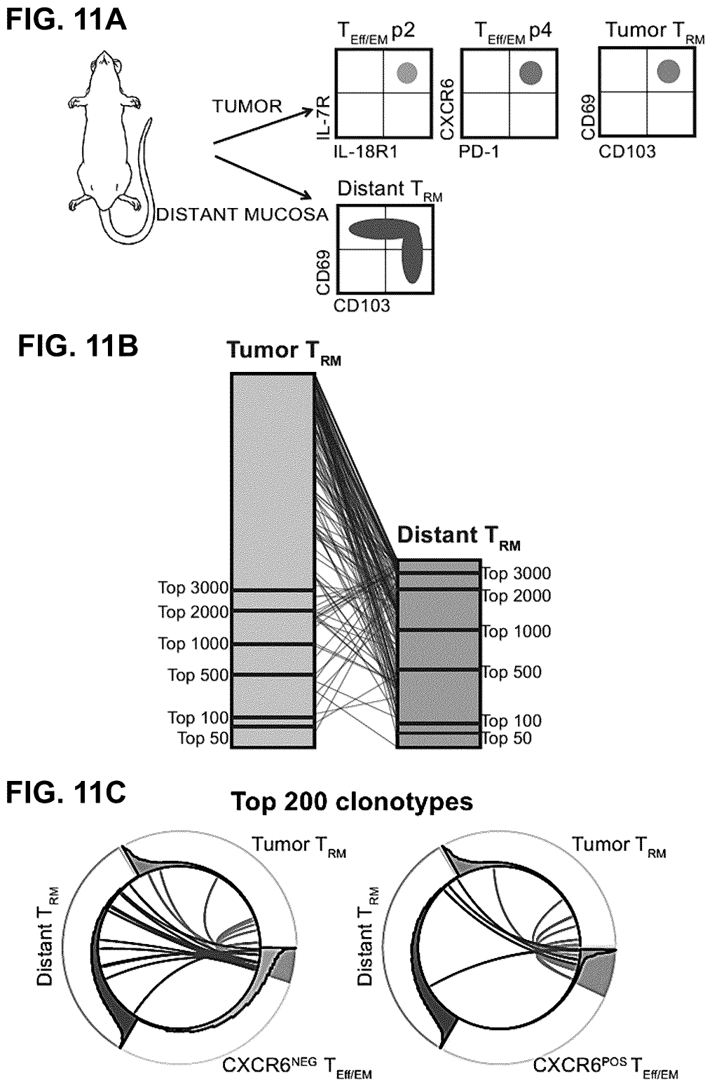

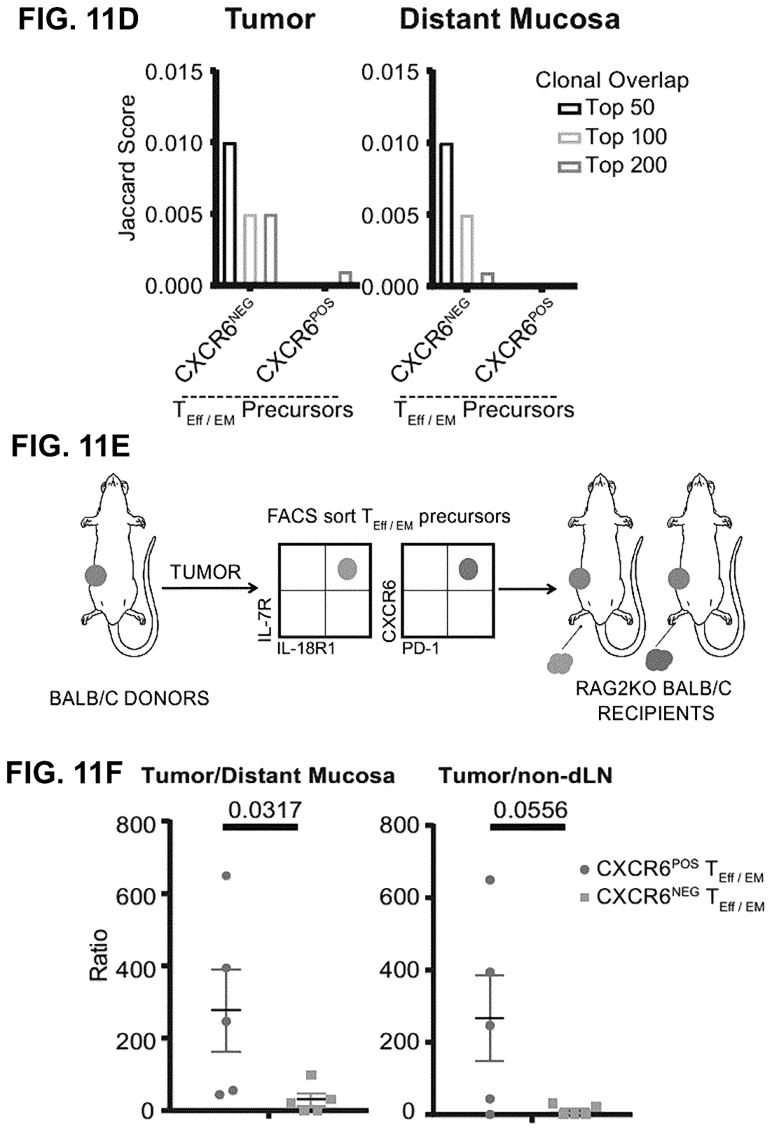

Since CXCR6 and S1PR1 were reciprocally expressed in TEff/EM cells, TCRβ repertoire sequencing was performed with the purified TEff/EM subpopulations to trace the lineage relationship between distinct intra-tumor TEff/EM populations and distant TRMs. Tumor CD44hiCD103−CXCR6+PD−1+TEff/EM cells and CD103−IL7R+IL18R1+TEff/EM cells as well as tumor and distant TRMs (CD103+CD69+) were all sorted for repertoire analysis and clonotype lineage tracing. (FIG. 11A and Table 3). The repertoire overlapped between the purified tumor TRMs and distant TRMs was compared. As previously found in bulk tissue repertoire analysis (FIG. 1I), both high and low frequency TRM clonotypes in these two compartments were shared (FIG. 11B), supporting that tumor and distant TRMs arose from a common precursor population. In addition, both CXCR6−IL7R+IL18R1+ and CXCR6+PD−1+subsets shared TCR clonotypes with tumor TRMs (FIG. 11C and FIG. 11D). However, for those highly expanded TEff/EM clones, only the CXCR6-subset contributed to the formation of distant TRMs, while CXCR6+clonotypes barely overlapped with distant TRMs. This indicated that CXCR6 may serve as a retention signal to keep T_{RM} precursors in the tumor.

TABLE 3
QC Analysis of TCRb Repertoire Sequencing
Samples Related to FIG. 11A-FIG. 11F.

| | Cells | Total Mapped | Unique TCR | Mean Read |
|---|---|---|---|---|
| Sample ID | Sorted | Reads | Clonotypes | Per TCR |
| CXCR6+ | 8769 | 21410 | 6414 | 3.34 |
| CXCR6− | 9674 | 15129 | 5529 | 2.74 |
| Tumor Trm | 13000 | 113246 | 11730 | 9.65 |
| Distant Trm | 11600 | 15554 | 4074 | 3.82 |

To validate this tumor-retention mechanism, CXCR6⁺PD-1⁺ and IL7R⁺IL18R1⁺ precursor populations from the tumors of CD45.1⁺congenically marked Balb/c mice were sorted and then equal numbers (15,000-25,000) were intratumorally transferred into a 4T1 tumor growing in the mammary tissue of Rag2KO Balb/c mice (FIG. 11E). Two weeks after transfer, transferred T_{Eff/EM} precursor cells were recovered from the tumor, distant mucosa, and non-draining inguinal lymph node and then subjected to FACS analysis. The ratio of cells recovered in the tumor vs. the distant mucosa was calculated as the readout to minimize experimental variations. The recovered ratios of cells in the tumor vs. non-draining lymph node were included as a reference. When compared to CXCR6⁻ cells, the CXCR6⁺TER/EM cells were retained in the tumor, a finding which validated the preference of the T_{Eff/EM} p4 population to stay in the tumor to become tumor T_{RM}s (FIG. 11F).

### Example 7

### Breaking CXCR6-Mediated Retention Enhances Protection Against Distant Tumor Metastasis

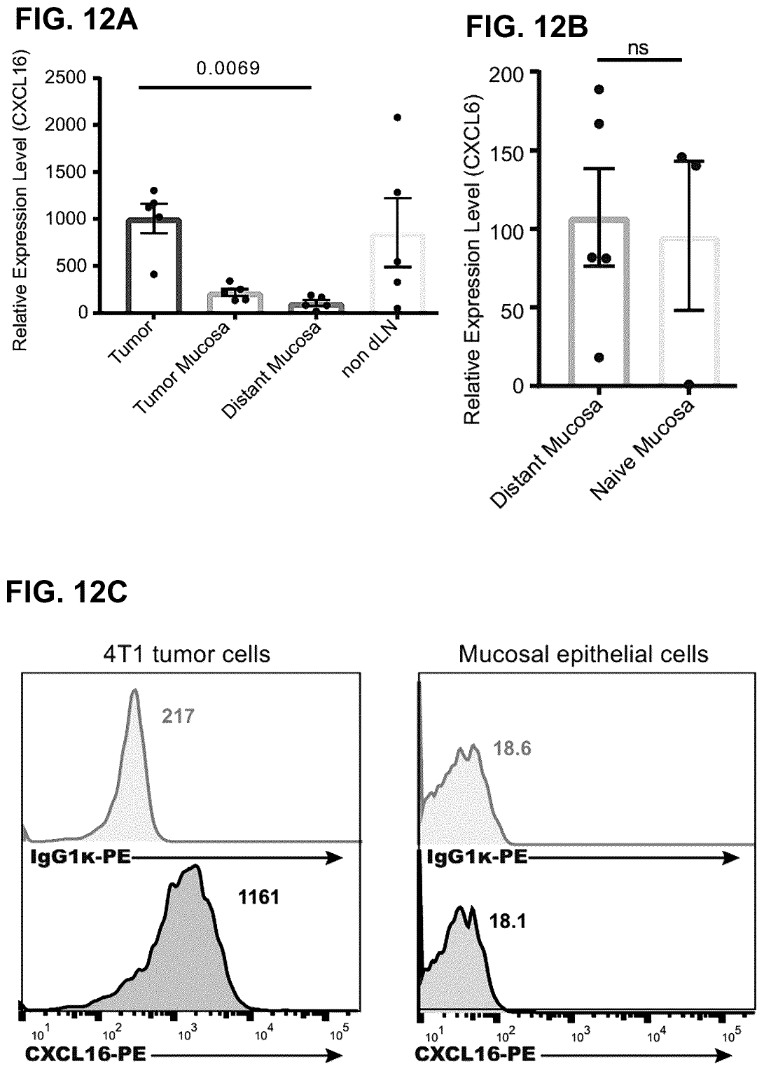

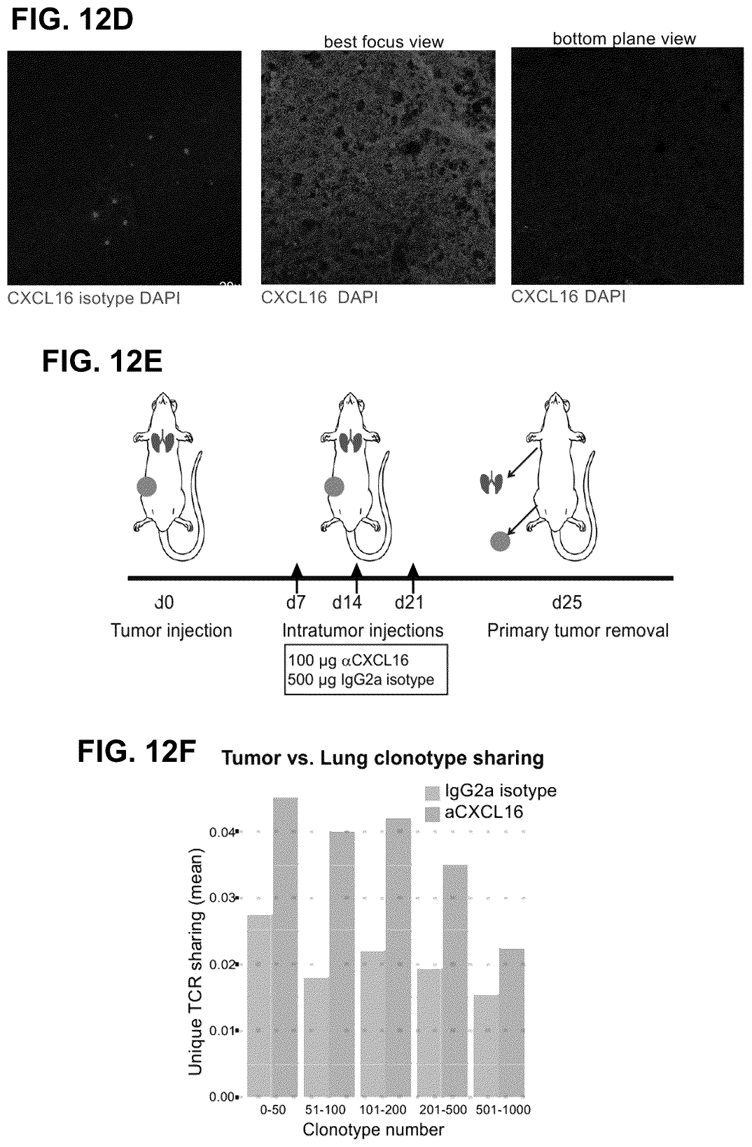

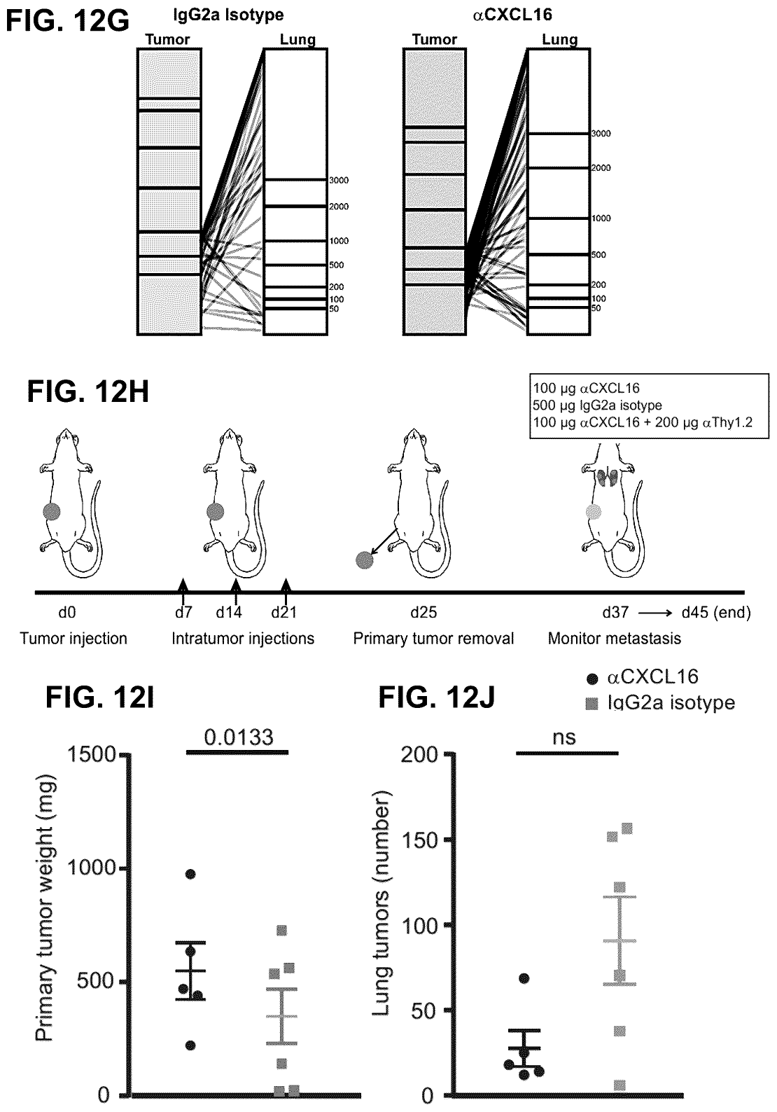

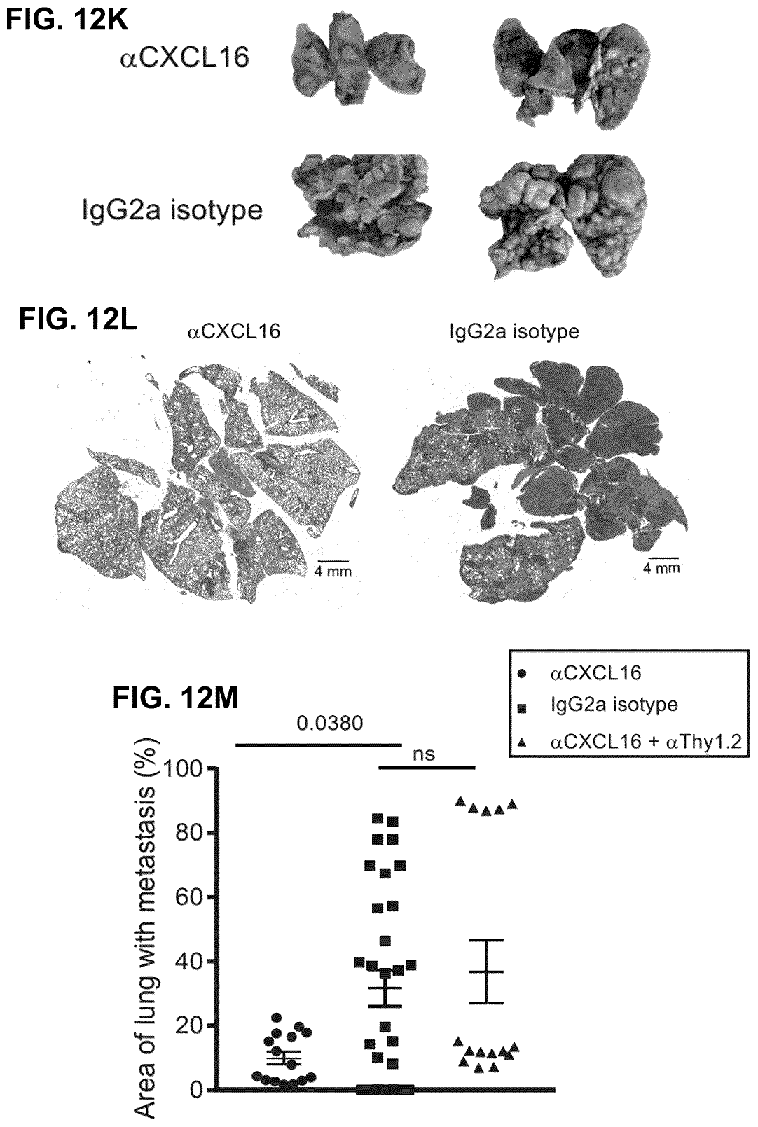

Since CXCR6 is the receptor that chemoattracts T_{Eff/EM} cells in the tumor, its sole ligand, CXCL16, was predicted to be expressed in the tumor tissue. Therefore, qPCR analysis was performed for Cxcl16 mRNA with tumor, tumor-adjacent mucosa, distant mucosa, and non-draining lymph node tissues isolated from 4T1 tumor-bearing mice. Mammary gland mucosa of tumor-naïve mice served as a control. Compared to that in mucosal tissues, Cxcl16 expression in the tumor was significantly stronger (FIG. 12A). Further, the relative Cxcl16 expression level between the distant mucosa from tumor-bearing mice was comparable to that in the tumor-naïve mucosa (FIG. 12B). Flow cytometry staining confirmed that 4T1 tumor cells were a direct source of CXCL16 production (FIG. 12C). This was further validated by confocal microscopy: within the 4T1 tumor tissues, CXCL16 was almost universally expressed on the surface of 4T1 tumor cells (FIG. 12D, best focus view). In addition, produced CXCL16 proteins were deposited on the surrounding extracellular matrix (FIG. 12D, bottom plane view). These data indicated that the 4T1 tumor microenvironment strongly attracted CXCR6⁺ T cells to stay.

Tumor-produced CXCL16 was determined to be a key retention molecule employed to curb the residency of tumor-specific T cells in distant tissues. Considering the sentinel functions of T_{RM}s, this retention could be a mechanism exploited by tumors to dampen immunity in distant tissues and facilitate the engraftment of metastases. To test this, at days 7, 14, and 21 after primary tumor inoculation, a CXCL16 antibody was intra-tumorally injected to neutralize CXCR6 binding (FIG. 12E). At day 25, the primary tumor was surgically removed and TCRβ repertoire sequencing was performed to characterize the difference in T cell infiltration in the pre-metastatic lung tissue following CXCL16 antibody blocking. The clonotype sharing was increased across T cells in all frequency categories (FIG. 12F). Zooming in on the high frequency clones, which were likely to be enriched by expanding tumor specific T_{Eff/EM} cells, the increase was also obvious (FIG. 12G). This indicated that breaking CXCR6-mediated retention in the tumor resulted in more T cells egressing to the distant lung tissue.

Whether promoting T cell infiltration to the lung could result in enhanced protection against tumor metastasis was next evaluated. Using the same experimental scheme detailed above, animals were monitored for 2-3 weeks following the removal of the primary tumor (FIG. 12H). Anti-CXCL16 treatment caused a moderate difference in the weight of the primary tumors (FIG. 12I). At the humane endpoint of individual mice, the spontaneous 4T1 metastases in the lung was assessed. Metastases on the surface of the lung were counted and found that the number of tumor nodules was not statistically different between control IgG and anti-CXCL16 treated mice (FIG. 12J). However, there were striking differences in metastatic tumor burden (FIG. 12K-FIG. 12J). To prove that this protective mechanism was T cell intrinsic, T cells were depleted with an anti-Thy 1.2 antibody while anti-CXCL16 was co-administered to the mice (FIG. 12H). The anti-CXCL16-enhanced protection was lost when T cells were depleted (FIG. 12M). This indicated that breaking CXCL16-CXCR6 mediated T cell retention in primary breast tumors fortified anti-metastatic immunity in the lung.

### Example 8

### Combinatorial Neoadjuvant Therapy Enhanced Protection Against Tumor Metastasis

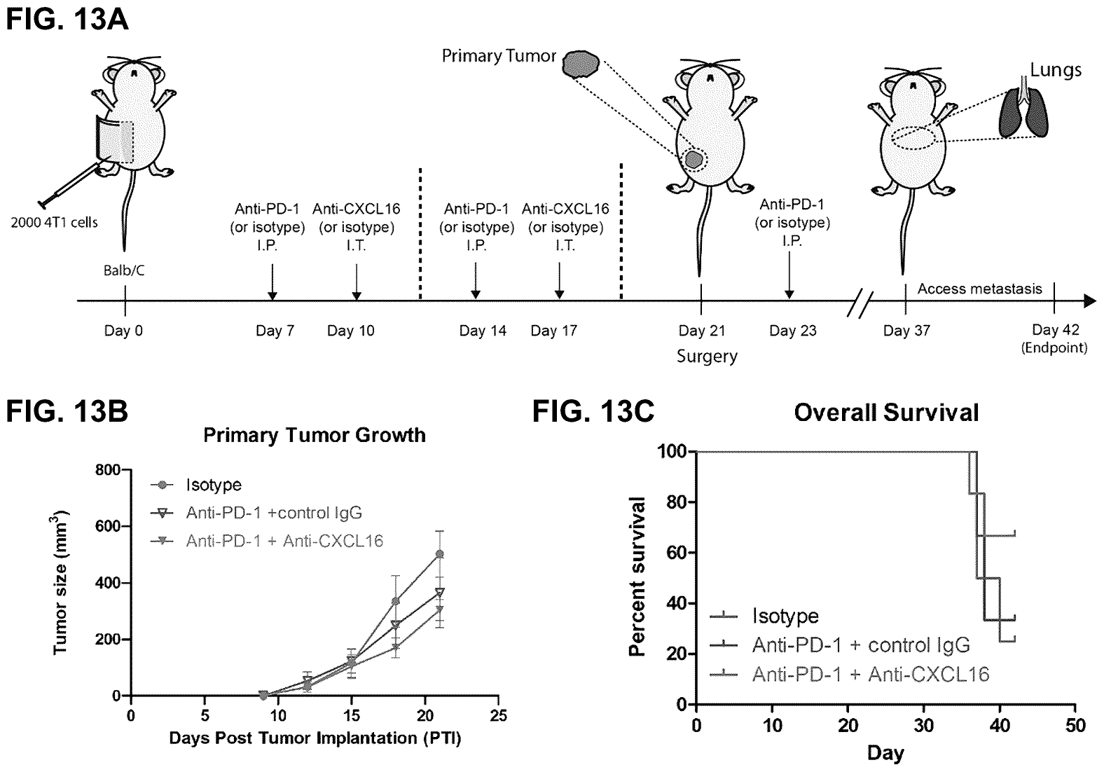

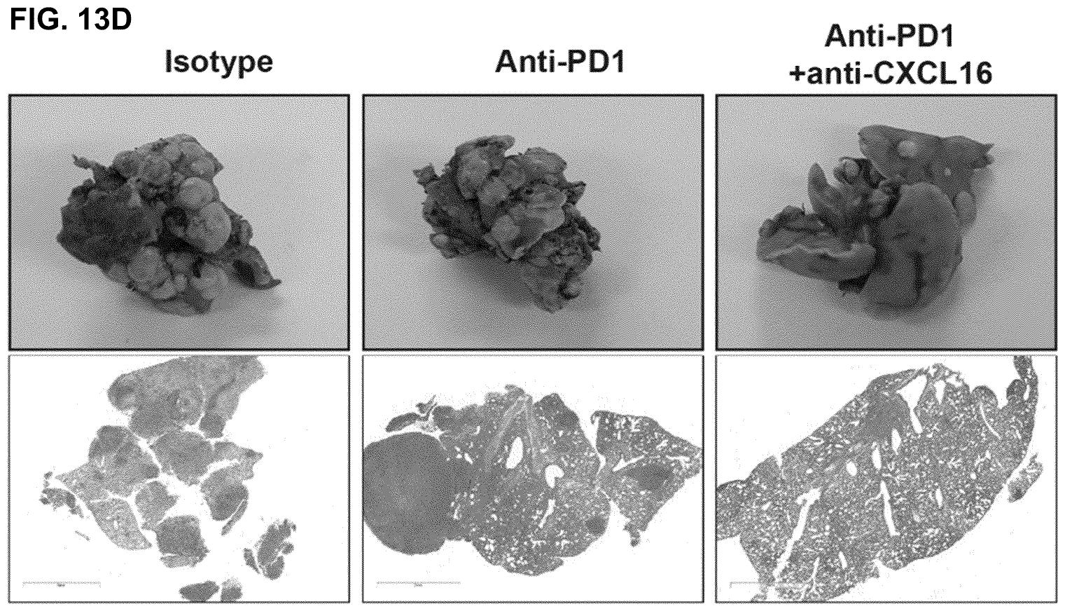

Next, whether the combination of anti-CXCL16 therapy with another therapy was effective against tumor metastasis was explored. As FIG. 13A-FIG. 13D show, a combinatorial neoadjuvant therapy enhanced protection against tumor metastasis. FIG. 13A shows the pre-clinical neoadjuvant therapy schema for the combination of anti-PD1 and anti-CXCL16. Prior to surgery, anti-PD1 therapy (delivered intraperitoneally) and anti-CXCL16 therapy (delivered intratumorally) were administer to mice to boost the proliferation of PD1+CXCR6+ precursor cells. These precursors were released from the tumor site by blocking CXCL16, the ligand of CXCR6. After surgical resection of the primary tumor (on day 21), the lung metastasis and overall survival were assessed. Day 42 was the endpoint of the study.

FIG. 13B shows the growth of primary tumors before the surgical removal. As the mono-drug, anti-PD1 alone failed to deliver efficacy to 4T1 tumor growth. The anti-PD1 and anti-CXCL16 combinatory treatment had moderate efficacy on the progression of primary tumors as measured across 21 days. FIG. 13C shows the overall survival of mice through the neoadjuvant therapy. Lung metastasis was the cause of death. As the mono-drug, anti-PD1 alone failed to prolong the survival of post-surgery mice. The anti-PD1 and anti-CXCL16 combinatory treatment increased the overall survival ratio. Finally, FIG. 13D shows representative images of 4T1 tumor lung metastases harvested between days 16 to 21 post-surgery. The top panels show the surface view of 4T1 tumor nodules while the bottom panels show the cross-section of the whole lung after HE staining.

### Summary of Specific Examples

In an orthotopic breast cancer model, T_{RM}s developed in remote mammary gland tissues at early stages of tumorigenesis. The TCR repertoire sequencing data revealed that clonotypically, these T_{RM}s were generated in the pre-metastatic stage from the same precursor cells which developed T_{EM}s and T_{RM}s within the tumor (FIG. 11B) as well as T_{RM}s in the tumor-adjacent mucosal tissue (FIG. 1I). Accordingly, dominant TCR clones in the mammary mucosa were shared among individual tumor-bearing mice. These TCRs were also shared between the tumor and tumor-distant mucosa but were distinct from mammary gland T_{RM}s in tumor-naïve mice. Single cell RNA-seq showed that a few tumor T_{EM}s shared a similar transcriptome with active T_{RM}s, although they were sorted based on their classical surface markers. The transcriptomic similarity between tumor T_{EM}s and T_{RM}s echoed the epigenetic similarity recently identified between these two populations in an LCMV infection model, especially for genomic loci that have the most dynamic changes through naïve to memory differentiation (Fonseca, R., et al. (2020) Nature Immunology). This transcriptomic similarity was reinforced by the pseudotime analysis. During the synthetic developmental process, the transcriptomic transition that generated terminally differentiated T_{EM}s was highly similar to the one begetting active T_{RM}s. Pseudotime analysis further showed that, although they were sorted from tumors, a few CD44^{hi}CD62L⁻CD103⁻ cells had already developed a transcriptome resembling that of CD103⁺ T_{RM}s purified from the distant mucosa. These results together indicated that distant mucosa T_{RM}s were generated from T_{Eff/EM} precursors that developed within the tumor. Before gaining their tumor resident credentials, these precursors were able to egress. After they circulated to and infiltrated the distant mammary gland, they found their necessary niche in the local tissue environment, which harbored and supported them to finish their final differentiation to become T_{RM}s in the remote tissue.

The generation of T_{RM}s in remote tissues was a protective mechanism against metastasis. Before metastatic tumor cell invasion, T_{RM}s established their defensive perimeters in distant tissues. Distant T_{RM}s function acted as perfect sentinels. On one hand, T_{RM}s are derived from T_{Eff/EM} precursors and have tumor antigen specificity, allowing them to detect tumor cells upon their arrival. On the other hand, as memory cells residing in the tissue, they can be reactivated quickly to expedite immune responses locally. These features are especially important for malignancies like breast cancer for which the 5-year survival rate in the US is 99% if only localized tumors are found. This rate drops to 27% if there are distant metastases (NCI SEER program). While the importance of T_{RM}s in various types of epithelial-associated cancers has recently emerged (Amsen, D., et al. (2018) Nat Immunol 19, 538-546), their exact anti-tumor roles were not characterized. A clinical study on early-diagnosed patients with local gastric cancer showed that their 4-year prognosis was associated with the clonal diversity of T_{RM}s in the tumor-adjacent mucosa rather than the diversity of T cells in the tumor or peripheral blood. The majority of the mortality in this cohort was the result of stomach cancer relapse or liver or peritoneum metastasis (Jia, Q., et al. (2015) Oncoimmunology 4, e1001230). Although the distant mucosal tissues of those patients could not be surveyed, based on the repertoire similarity between the tumor-adjacent and tumor-distant mucosa, the correlation between prognosis and T_{RM} diversity can reflect the unique immunosurveillance function of extratumoral T_{RM}s. Their protective functions go beyond the primary tumor and are specialized against tumor recurrence.

To approach the retention mechanism separating T cells that stay in the tumor versus those that leave to remote tissues, CXCR6 was identified as a key player. CXCR6 was proposed to be the chemokine receptor driving flu-specific T_{RM}s to reside in the airway epithelium, while CXCR6⁻ T_{RM}s stayed in the interstitium (Wein, A. N., et al. (2018) Journal of Experimental Medicine, jem.20181308). In the same influenza infection model, fully-differentiated CXCR6⁺ T_{RM}s in the interstitium replenish the airway compartment, not circulating T_{EM}s (Ely, K. H., et al. (2006) Journal of Immunology 176, 537-543; Takamura, S., et al. (2019) Journal of Experimental Medicine, jem.20190557; Zammit, D. J., et al. (2006) Immunity 24, 439-449). Both studies clearly demonstrated that airway CXCL16 production and T_{RM} CXCR6 expression were necessary for airway T_{RM}s to form. The data presented herein revealed a similar chemo attraction mechanism trapping effector T cells in the tumor. The heightened expression of CXCL16 by tumor cells is a common characteristic found in tissue from gastric (Xing, Y. N., et al. (2012) Human pathology 43, 2299-2307) and colon carcinoma patients (Hojo, S., et al. (2007) Cancer Research 67, 4725-4731) as well as in various mammary carcinoma cell lines (Meijer, J., et al. (2008) Cancer Research 68, 4701-4708). This differential expression was also observed in the 4T1 tumor versus the distant mammary gland mucosa or the mucosa from tumor naïve mice. Transferred CXCR6+tumor T_{Eff/EMS} were more likely to be trapped in the recipient tumor than their CXCR6 counterparts. Furthermore, in a snapshot of TCR repertoire analysis, more tumor CXCR6-T_{Eff/EM} TCRs overlapped with T_{RM}s isolated from the distant mucosa than CXCR6⁺T_{Eff/EMS} in the tumor. In this 4T1 model, tumor cells served as the source of CXCL16 to retain tumor-infiltrating T_{Eff/EM} cells. The repertoire analysis also showed that TCRs from CXCR6⁻ and CXCR6⁺ subsets had significant overlap. Whether CXCR6 expression is stochastic or how it is regulated in tumor T_{Eff/EM} cells is still not clear. However, the scRNA-seq and FACS data demonstrated a strong correlation between CXCR6 and PD-1 expression, which indicated that it could be related to tumor antigen stimulation.

These studies showed that T_{RM}s, especially T_{RM}s in the distant mammary gland divided into two major populations based on their ribosome-associated gene expression. The elevated expression of these genes was closely related to quiescent features of their transcriptome. This association has been revealed in CD8⁺ memory T cell development during LCMV infection. In this model, both ribosomal protein mRNAs and overall translation activities are drastically decreased when T cells enteres the phase of terminal effector differentiation. Ribosomal protein mRNA expression in T_{Eff}S was lower than that in memory precursor (T_{MP}) cells and their expression in T_{MP}s was lower than in quiescent naïve T cells (Araki, K., et al. (2017) Nature immunology 18, 1046-1057). Furthermore, suppressed ribosomal protein mRNA expression depended on antigen stimulation and mTORC1 activity. The mTORC1 inhibitor rapamycin was shown to promote long-lived T_{CM} formation (Araki, K., et al. (2010) Immunological reviews 235, 234-243). This mechanism could be directly applied to T_{RM}s. Like LCMV Clone 13-induced exhaustion, chronic tumor antigen stimulation may suppress ribosomal protein mRNA expression. Consequently, T_{RM}s found in the distant mucosa were isolated from tumor antigens and thus returned to a quiescent stage to cope with their longevity.

The Examples demonstrated several key points. First, T_{RM}s in tumors and distant tissues are developed from T_{Eff/EM} precursor cells. Second, T_{RM}s comprise active and quiescent subsets that resemble T_{EM}s and T_{CM}s. Since they resemble T_{CM}s, they are long-lived. Third, precursors of tumor-specific T_{RM}s egress the tumor tissue, reach distant tissue. and developed into distant tissues T_{RM}s prior to tumor metastasis. Fourth, these tumor-derived T_{RM}s, therefore, are tumor specific and function to detect and defend against metastatic tumor cells. This represents the first evidence for a distant tumor-specific T cell protection against metastasis. And, fifth, CXCL16 in tumor microenvironment traps T_{RM} precursors to facilitate metastasis

1. A method of reducing the risk of developing metastases, the method comprising:
administering to a subject in need thereof a therapeutically effective amount of an interfering molecule that disrupts and/or prevents the physical interaction of CXCL16 with CXCR6.

2. The method of claim 1, wherein the interfering molecule comprises a peptide, a phosphopeptide, peptide fragment, an antibody, an antisense or small interfering RNA, a small molecule, or a combination thereof.

3. The method of claim 1, wherein the interfering molecule comprises an anti-CXCL16 antibody, an anti-CXCR6 antibody, or a combination thereof,

4. The method of claim 3, wherein the administered dose of the anti-CXCL 16 antibody, the anti-CXCR6 antibody, or the combination thereof comprises about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

5. The method of claim 1, wherein administering the interfering molecule comprises oral administration, intravenous administration, intratumoral administration, intraperitoneal administration, or any combination thereof.

6. The method of claim 1, further comprising administering to the subject an anti-PD1 molecule.

7. The method of claim 6, wherein the anti-PD1 molecule comprises an anti-PD1 antibody, an anti-PDL1 antibody, or any combination thereof.

8.-9. (canceled)

10. The method of claim 6, wherein the anti-PD1 molecule is administered prior to, concurrent with, or after the administration of the interfering molecule.

11.-14. (canceled)

15. The method of claim 1, further comprising monitoring the subject for adverse effects.

16.-19. (canceled)

20. The method of claim 1, further comprising administering to the subject one or more additional anti-cancer therapies.

21. The method of claim 20, wherein the one or more anti-cancer therapies comprises endocrine therapy, radiotherapy, hormone therapy, gene therapy, thermal therapy, ultrasound therapy, or any combination thereof.

22. The method of claim 20, wherein the one or more anti-cancer therapies comprise one or more chemotherapeutic agents.

23. The method of claim 22, wherein the one or more chemotherapeutic agents comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof.

24. (canceled)

25. The method of claim 1, wherein the subject has been diagnosed with ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

26. The method of claim 1, wherein disrupting and/or preventing the physical interaction of CXCL16 with CXCR6 breaks CXCR6-mediated retention of precursors of resident memory T cells in a primary tumor.

27. The method of claim 26, wherein breaking CXCR6-mediated retention of precursors of resident memory T cells prevents or inhibits metastasis of cancer cells.

28. The method of claim 27, wherein preventing or inhibiting metastasis of cancer cells comprises allowing precursors of tumor-specific TRMs to egress the tumor or tumor tissue, travel to distant or non-tumor tissue, and develop into TRMs.

29. The method of claim 1, further comprising surgically resecting a tumor from the subject.

30. (canceled)

31. The method of claim 1, further comprising subjecting the subject to one or more invasive or non-invasive diagnostic assessments.

32. The method of claim 31, wherein the one or more non-invasive diagnostic assessments comprise x-rays, computerized tomography (CT) scans, magnetic resonance imaging (MRI) scans, ultrasounds, positron emission tomography (PET) scans, or any combination, and wherein the one or more invasive diagnostic assessments comprise a tissue biopsy or exploratory surgery.

33.-45. (canceled)
